(19) United States
(12) Patent Application Publication
(10) Pub. No.: US 2004/0005554 A1
(43) Pub. Date: Jan. 8, 2004

(54) Novel glycoproteins and methods of use thereof

(76) Inventors: Nabil El Tayar, Milton, MA (US); Robert K. Campbell, Wrentham, MA (US); Chistie A. Kelton, Hopkinton, MA (US); Chaomei He, Wellesley, MA (US)

Correspondence Address: LAHIVE & COCKFIELD, 28 STATE STREET, BOSTON, MA, 02109, US

(21) Appl. No.: 09/927,876

(22) Filed: Aug. 10, 2001

Related U.S. Application Data
(63) Continuation of application No. 09/851,465, filed on May 8, 2001, now abandoned.
(60) Provisional application No. 60/202,724, filed on May 8, 2000.
(60) Provisional application No. 60/225,035, filed on Aug. 11, 2000.

(51) Int. Cl. C12Q 1/68; C07H 21/04; A61K 48/00; C07K 16/40; C12P 21/02; C12N 5/06; A61K 39/395; C12N 9/64; A61K 38/48

(52) U.S. Cl. 435/6; 435/69.1; 435/226; 435/320.1; 435/325; 514/44; 424/146.1; 424/94.63; 530/388.26; 536/23.2

(57) ABSTRACT

The present invention provides novel isolated ARP/BRP polynucleotides and the membrane-associated or secreted polypeptides encoded by the ARP/BRP polynucleotides. Also provided are ARP and BRP protein multimers. Further provided are the antibodies that immunospecifically bind to a ARP/BRP polypeptide or any derivative, variant, mutant or fragment of the ARP/BRP polypeptide, a ARP/BRP multimer polynucleotide or antibody. The invention additionally provides methods in which the ARP/ BRP polypeptide, multimer, polynucleotide and antibody are utilized in the detection and treatment of a broad range of pathological states, e.g. reproductive disorder, as well as to other uses.

### RELATED APPLICATIONS

This application claims priority from Applications U.S. Ser. No. 60/,225,035 filed Aug. 11, 2000 and U.S. Ser. No. 09/851,465 filed May 8, 2001, which claims priority from U.S. Ser. No. 60/202,724 filed May 8, 2000, which are incorporated by reference in their entirety.

### FIELD OF THE INVENTION

The invention relates to polynucleotides and polypeptides encoded by such polynucleotides, as well as vectors, host cells, antibodies and recombinant methods for producing the polypeptides and polynucleotides.

### BACKGROUND OF THE INVENTION

Glycoprotein hormones, especially those initially found to be synthesized and secreted by the anterior pituitary gland, can play important roles in a variety of physiological functions. These functions can include, e.g., metabolism, temperature regulation, growth, and reproduction. The pituitary glycoproteins, luteinizing hormone (LH), follicle stimulating hormone (FSH), and thyroid stimulating hormone (TSH) are similar in structure to chorionic gonadotropin (hCG), a placental gonadotropin. These hormones belong to the cystine knot family of proteins and form a multimer of an alpha and a beta subunit. Within a species the alpha chain for each of the known hormones is identical. The beta chain in contrast, varies in sequence and confers the specificity to a given hormone.

### SUMMARY OF THE INVENTION

The invention is based, in part, upon the discovery of novel polynucleotide sequences encoding novel beta and alpha subunits of a glycoprotein. The encoded proteins have been named beta related protein (BRP) or alpha related protein (ARP) respectively. Collectively these polynucleotides and polypeptides are refered to herein as ARP/BRP.

In one aspect, the present invention provides isolated nucleic acid molecules (SEQ ID NO: 1 and 3, as shown in FIG. 1) that encode a beta related polypeptide (BRP), or fragment, homolog, analog or derivative thereof. The nucleic acid can include, e.g., nucleic acid sequence encoding a polypeptide that is at least 75% identical to the polypeptides of FIG. 2 (SEQ ID NO: 2 and SEQ ID NO: 4). The nucleic acid can be, e.g., a genomic DNA fragment, or it can be a cDNA molecule.

The invention also provides a protein multimer, e.g. multimer, of a first polypeptide and a second polypeptide. The first polypeptide can be an ARP or BRP polypeptide.

The second polypeptide can be an apha glycoprotein subunit or a beta glycoprotein subunit, ARP or BRP. Alternatively, the second polypeptide can be a cystine knot protein.

Also included in the invention is a vector containing one or more of the nucleic acid molecules described herein, and a cell containing the vectors or nucleic acids described herein.

The present invention is also directed to host cells transformed with a vector comprising a ARP/BRP nucleic acid molecule.

The present invention provides a method of inducing an immune response in a mammal against a polypeptide encoded by any of the nucleic acid molecules or protein multimers disclosed herein by administering to the mammal an amount of the polypeptide sufficient to induce the immune response.

In a further aspect, the invention provides an antibody that binds specifically to a ARP, BRP or a hetero- or homo-multimer of these or multimers of these with alpha or beta subunits from other gonadotrophins. The antibody can be, e.g., a monoclonal or polyclonal antibody, and fragments, homologs, analogs, and derivatives thereof. The invention also includes a pharmaceutical composition including a ARP/BRP antibody and a pharmaceutically acceptable carrier or diluent. The present invention is also directed to isolated antibodies that bind to an epitope on a polypeptide encoded by any of the nucleic acid molecules described above.

In one aspect, the invention includes a pharmaceutical composition that includes a ARP/BRP nucleic acid and a pharmaceutically acceptable carrier or diluent. In a further aspect, the invention includes a substantially purified ARP/BRP polypeptide, e.g., any of the ARP/BRP polypeptides encoded by a ARP/BRP nucleic acid, and fragments, homologs, analogs, and derivatives thereof. In another aspect, the invention includes a pharmaceutical composition that includes a ARP/BRP multimer and a pharmaceutically acceptable carrier or diluent. The invention also includes a pharmaceutical composition that includes a ARP/BRP polypeptide and a pharmaceutically acceptable carrier or diluent.

The present invention is further directed to kits comprising antibodies that bind to a polypeptide encoded by any of the nucleic acid molecules described above and a negative control antibody.

The invention further provides a method for producing a ARP/BRP polypeptide. The method includes providing a cell containing a ARP/BRP nucleic acid, e.g., a vector that includes a ARP/BRP nucleic acid, and culturing the cell under conditions sufficient to express the ARP/BRP polypeptide encoded by the nucleic acid. The expressed ARP/BRP polypeptide is then recovered from the cell. The cell can be, e.g., a prokaryotic cell or eukaryotic cell. Preferably, a higher eukaryotic cell, e.g., mammalian.

The invention further provides a cell expressing ARP/BRP or multimers of these polypeptides at a modified level with respect to the wild type cell, from an endogenous sequence, after the insertion of a non-native regulatory element and or insertion of an amplifiable genein operable connection to the endogenous gene sequence.

The present invention is also directed to methods of identifying a compound that binds to ARP/BRP polypeptide or multimer by contacting the ARP/BRP polypeptide or multimer with a compound and determining whether the compound binds to the ARP/BRP or multimer polypeptide.

The present invention is also directed to compounds that modulate ARP/BRP polypeptide or multimer activity identified by contacting a ARP/BRP polypeptide or multimer with the compound and determining whether the compound modifies activity of the ARP/BRP polypeptide or multimer, binds to the ARP/BRP polypeptide or multimer, or binds to a nucleic acid molecule encoding a ARP/BRP polypeptide.

In another aspect, the invention provides a method of determining the presence of or predisposition of a reproductive disorder such as ovulatory disorders or infertility in a subject. The method includes providing a protein sample from the subject and measuring the amount of ARP/BRP polypeptide or multimer in the subject sample. The amount of ARP/BRP in the subject sample is then compared to the amount of ARP/BRP polypeptide or multimer in a control protein sample. An alteration in the amount of ARP/BRP polypeptide or multimer in the subject protein sample relative to the amount of ARP/BRP polypeptide or multimer in the control protein sample indicates the subject has a reproductive disorder. A control sample is preferably taken from a matched individual, i e., an individual of similar age, sex, or other general condition but who is not suspected of having a reproductive disorder. Alternatively, the control sample may be taken from the subject at a time when the subject is not suspected of having a reproductive disorder. In some embodiments, the ARP/BRP polypeptide or multimer is detected using a ARP/BRP antibody.

In another aspect, the invention provides a method of determining the presence of or predisposition to a reproductive disorder such as ovulatory disorders or infertility in a subject. The method includes providing a nucleic acid sample, e.g., RNA or DNA, or both, from the subject and measuring the amount of the ARP/BRP nucleic acid in the subject nucleic acid sample. The amount of ARP/BRP nucleic acid sample in the subject nucleic acid is then compared to the amount of ARP/BRP nucleic acid in a control sample. An alteration in the amount of ARP/BRP nucleic acid in the sample relative to the amount of ARP/BRP in the control sample indicates the subject has a reproductive disorder.

In another aspect, the invention provides a method of treating a pathological state, e.g., reproductive disorder in a subject. The method includes administering a ARP/BRP polypeptide, multimer or antibody to a subject in an amount sufficient to alliviate the pathological condition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a representation of the nucleotide sequences (SEQ ID NO: 1 and 3) of novel beta related proteins according to the invention.

FIG. 2 is a representation of the translated amino acid sequence (SEQ ID NO: 2 and 4) of novel beta related proteins according to the invention.

FIG. 3 is a representation of the predicted signal sequence of the beta related protein according to the invention. (SEQ ID NO: 10).

FIG. 4 is a representation of the region of Gen Bank Accesion NO: AL118555 containing the genomic coding sequence and translation product. Exons are in bold.

FIG. 5 is an alignment of a human BRP polypeptide sequence (also called beta5; SEQ ID NO: 2) with LHβ (SEQ ID NO: 6); FSHβ (SEQ ID NO: 7); CGβ (SEQ ID NO: 8) TSHβ (SEQ ID NO: 9).

FIG. 6 illustrates BRP sequence identities (% value above diagonal) compared to other glycoproteins hormone beta subunits and similarities (% value below diagonal). Analysis made using BLAST and represent identities and similarities occuring across the core mature sequence (i.e., from cys 9 [hCG numbering) to cys 100).

FIG. 7 is a representive model of the structure of BRP. Panel A illustrates the absence of a seat belt domain. Panel B illustrates the glycosylation postions on BRP as compared to hCG and FSH.

FIG. 8 illustrates the Kyte-Doolittle hydrophobicity plot of human BRP.

FIG. 9 illustrates the Hopp-Woods hydrophobicity plot of human BRP.

FIG. 10 illustrates a BRP-hCG fusion protein according to the invention. Amino acids in bold represents amino acid sequence derived from hCG. Amino acids underlined represents amino acid sequence derived from ARP/BRP.

FIG. 11 illustrates BRP-hFSH fusion protein according to the invention. Amino acids in bold represent amino acid sequences derived from hFSH. Amino acids underlined represents amino acid sequence derived from ARP/BRP.

FIG. 12 is a representation of the nucleotide sequences (SEQ ID NO: 17, 19, and 21) of novel alpha related proteins according to the invention.

FIG. 13 is a representation of the translated amino acid sequence (SEQ ID NO: 18, 20 and 22) of novel alpha related proteins according to the invention. “\hat{ }” designates the predicted start site of the mature protein.

FIG. 14 is a representation of an extended genomic fragment of chromosome 11 (Homo sapiens Chromosome 11q13 BAC Clone b79g17, GenBank accession number AC000159); SEQ ID NO: 23).

FIG. 15 is an alignment of the human ARP polypeptide sequence (SEQ ID NO: 18) with FSHα (SEQ ID NO: 26) and FSHβ (SEQ ID NO: 27). Red letter indicates an identical residue in hARP found in hFSHa or hFSHb. Yellow denotes cysteine.

FIG. 16 is a representation of the region of Gen Bank Accesion NO: AC000159 (ARP) containing the genomic coding sequence and translation product. Exons are in bold and underlined, polyadenylation signal is underlined.

FIG. 17 is a representation of a northern blot analysis of ARP mRNA.

FIG. 18 is a representation of a multi-tissue expression (MTE) blot analysis of Arp gene expresssion.

FIG. 19 is a drawing of the plasmid construct “hBRP in pCR4Blunt” (A) containing the BRP open reading frame. The DNA sequence and amino acid translation of the open reading frame are also shown (B).

FIG. 20 is a drawing of the plasmid construct “BRP-NTAP” (A) containing the BRP open reading frame without the secretory signal peptide. The DNA sequence and amino acid translation of the open reading frame are also shown (B).

FIG. 21 is a drawing of the plasmid construct “AP- BRP in pAPtag5” (A) containing the open reading frame that encodes the AP- BRP fusion protein. The DNA sequence and amino acid translation of a portion of AP and BRP (in boldface) are also shown (B).

FIG. 22 is a drawing of the plasmid construct “BRP-GFP in pcDNA3.1” that contains the open reading frame that encodes the BRP-GFP fusion protein.

FIG. 23 is a representation of the DNA sequence and amino acid translation of a the BRP-GFP fusion protein encoded by the plasmid “BRP-GFP in pcDNA3.1”.

FIG. 24 is a drawing of the plasmid construct “FLAG- BRP in pFLAGCMV-1” (A) containing the open reading frame that encodes the FLAG- BRP fusion protein. The DNA sequence and amino acid translation of the open reading frame are shown (B). The arrows indicate the components of the fusion protein. The amino acid sequence of BRP is in boldface.

FIG. 25 is a drawing of the plasmid construct “FLAG- BRP in pCEP4.”

FIG. 26 is a drawing of the plasamid construct “pBS-SKIIhARP.4” containing the ARP open reading frame. The DNA sequence and amino acid translation of the open reading frame are also shown (B).

FIG. 27 is a representation of the DNA sequence and corresponding translation of the ARP-Leu protein. The position of the single nucleotide difference that results in the ARP-Phe form is indicated.

FIG. 28 is a drawing of the plasmid construct “pBS-SKII hARP-Phe” (A) containing the open reading frame that encodes the ARP-Phe protein. The DNA sequence and amino acid translation are also shown (B).

FIG. 29 is a drawing of the plasmid construct “pEGFP-N2-ARP” that contains the open reading frame that encodes the ARP-GFP fusion protein.

FIG. 30 is a representation of the DNA sequence and amino acid translation of the ARP-GFP fusion protein encoded by the plasmid “pEGFP-N2-ARP”.

FIG. 31 is a drawing of the plasmid construct “pAPtag5(RI) ARP-Phe” (A) containing the open reading frame that encodes the AP-ARP-Phe fusion protein. The DNA sequence and amino acid translation of the open reading frame are shown (B). The arrows indicate the components of the fusion protein. The amino acid sequence of ARP is in boldface.

FIG. 32 is a drawing of the plasmid construct “FLAG-ARP-Phe in pCEP4” (A) containing the open reading frame that encodes the FLAG-ARP-Phe fusion protein. The DNA sequence and amino acid translation of the open reading frame are shown (B). The arrows indicate the components of the fusion protein. The amino acid sequence of ARP is in boldface.

FIG. 33 is a drawing of the plasmid construct “FLAG-ARP in pCEP4.

FIG. 34 is a representation of a western blot analysis of secreted ARP-GFP and BRP-GFP fusion proteins.

FIG. 35 is a representation of a western blot analysis of secreted FLAG- BRP and FLAG- ARP fusion proteins.

FIG. 36 is a representation of SDS-PAGE and western blot analysis of purified BRP protein.

FIG. 37. is a photomicrograph of rat testis showing that AP-BRP binds to testicular cells and can be displaced by FLAG-BRP. Panel a) AP alone, b) AP-BRP, c) AP-BRP plus 390 nM FLAG-BRP.

FIG. 38. is a photomicrograph of rat ovary showing that AP-tagged protein from the AP-BRP+FLAG-ARP-Phe co-transfection binds to ovarian cells (corpora lutea) and can be displaced by FLAG-BRP/His-ARP-Phe. Panel a) AP alone, b) AP-BRP+FLAG-ARP-Phe, c) AP-BRP+FLAG-ARP-Phe plus conditioned media from a FLAG-BRP/His-ARP-Phe co-transfection.

FIG. 39. is a photomicrograph of rat ovary showing that AP-tagged protein from the AP-ARP-BRP+FLAG-ARP-Phe co-transfection binds to ovarian cells (follicles) and can be displaced by FLAG-BRP/His-ARP-Phe. Panel a) AP alone, b) AP-BRP+FLAG-ARP-Phe, c) AP-BRP+FLAG-ARP-Phe plus conditioned media from a FLAG-BRP/His-ARP-Phe co-transfection.

FIG. 40. is a photomicrograph of rat testis showing that AP-tagged protein from the AP-BRP+FLAG-ARP-Phe co-transfection binds to testicular cells and can be displaced by FLAG-BRP/His-ARP-Phe. Panel a) AP alone, b) AP-BRP+FLAG- ARP-Phe, c) AP-BRP+FLAG-ARP-Phe plus conditioned media from a FLAG-BRP/His-ARP-Phe co-transfection.

FIG. 41 is a drawing of the plasmid construct “6Hisg-ARP-Phe in pCEP4int” (A) containing the open reading frame that encodes the 6Hisg-ARP-Phe fusion protein. The DNA sequence and amino acid translation of the open reading frame are shown (B). The arrows indicate the components of the fusion protein. The amino acid sequence of ARP is in boldface.

### DETAILED DESCRIPTION OF THE INVENTION

The invention is based in part on the discovery of novel nucleic acid sequences encoding polypeptides related to glycoprotein beta and alpha subunits. Polypeptides and nucleic acids of the invention related to beta subunit are refered to herein as beta-related protein (BRP), whereas the polypeptides and nucleic acids of the invention related to the alpha subunit are refered to herein as alpha-related proteins (ARP). When used herein “ARP/BRP” is meant to refer to both the beta-related and the alpha-related nucleic acids and polypeptides of the invention. Table 1 below delineates the sequence descriptors that are used herein.

TABLE 1

| | |
|---|---|
| SEQ ID | |
| NO: | SEQUENCE DESCRIPTOR |

| | |
|---|---|
| 1 | Human BRP Open Reading Frame (ORF) nucleic |
| | acid |
| 2 | Human BRP polypeptide sequence |
| 3 | Xenopus BRP Open Reading Frame (ORF) nucleic |
| | acid |
| 4 | Xenopus BRP polypeptide sequence |
| 5 | Human BRP fragment a.a. WEKPI |
| 6 | LHβ |
| 7 | FSHβ |
| 8 | CGβ |
| 9 | TSHβ |
| 10 | Human BRP signal sequence: |
| | MKLAFLLLGPMALLLLAGYGCLG |
| 11 | Human CGβ signal sequence: |
| | MEMFQGLLLLLLLSMGGTWA |
| 12 | Human BRP loop 2: |
| | ETWEKPILEPPYIEAHHRV |
| 13 | Human BRP-hGCβ fusion protein |
| 14 | Human BRP-hFSHβ fusion protein |
| 15 | Human BRP antigentic peptide |
| | CETWEKPILEPPYIEAHHRVC |
| 16 | Human BRP antigentic peptide |
| | ETWEKPILEPPYIEAHHRV |
| 17 | Human ARP Open Reading Frame (ORF) lacking |
| | exons |
| 18 | Human ARP polypeptide sequence |
| 19 | Murine ARP Open Reading Frame (ORF) lacking |
| | exons |
| 20 | Murine ARP polypeptide sequence |
| 21 | Rat ARP Open Reading Frame (ORE) lacking |
| | exons |
| 22 | Rat ARP polypeptide sequence |
| 23 | Human ARE genomic DNA |
| 24 | Human ARE fragment a.a. LHPFNV |
| 25 | Human ARE fragment a.a. LKKVKV |
| 26 | Human FSHα |
| 27 | Human FSHβ |
| 28 | Human ARP signal sequence: |
| | MPMASPQTLVLYLLVLAVTEAWG |
| 29 | Murine ARP signal sequence: |
| | MPMAPRVLLLCLLGLAVTEGHS |
| 30 | Rat ARP signal sequence: |
| | MPMAPRVLLFCLLGLAVTEGHG |

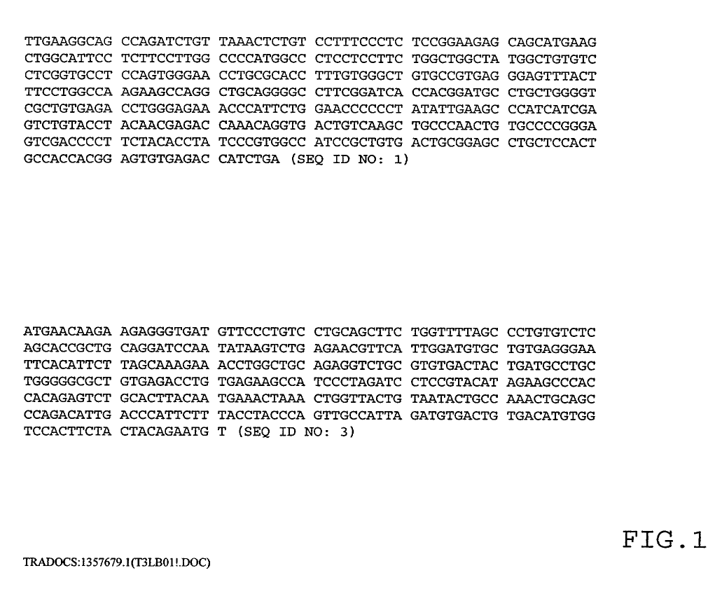

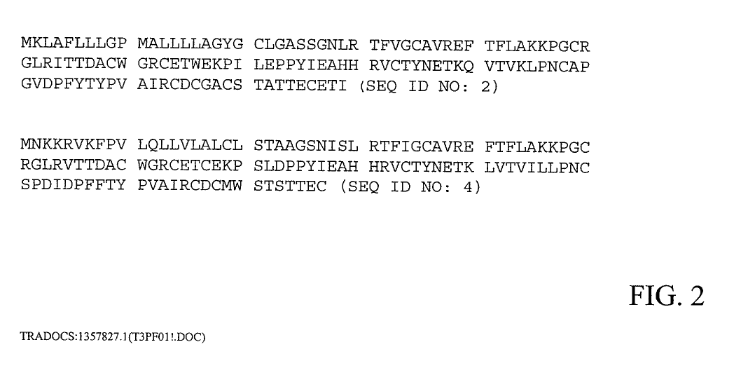

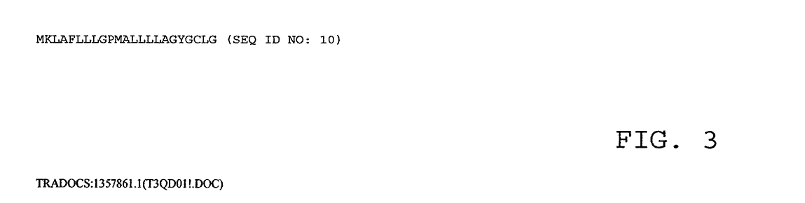

Included in the invention are nucleotide sequences encoding novel glycoprotein beta subunits. (see FIG. 1; SEQ ID NO: 1, and 3). The amino acid sequences of the encoded polypeptides are shown in FIG. 2 (SEQ ID NO: 2 and 4). GCG Spscan analysis predicted signal sequences as shown in FIG. 3. (SEQ ID NO: 10).

A nucleic acid encoding a BRP polypeptide was identified in a BAC containing genomic DNA sequence from chromosome 14. (GenBank Accession No. AL11855). An apparent full-length BRP coding region containing a translational start site and termination codon was identified in the BAC. The BRP coding region includes two exons and one intron. The BRP DNA sequence includes 387 nucleotides that encode a polypeptide of 129 amino acids (SEQ ID NO: 2).

The BRP nucleic acid sequence shows about 50% identity to the FSH beta subunit between the region coding the first to the last cysteine residue. The predicted mature coding region of the BRP protein shows 30-35% identity to the beta subunits of the glycoprotein family of hormones.

The presence of identifiable domains in ARP/BRP, proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, ARP/BRP were collected from the Conserved Domain Database (CDD) with Reverse Position Specific BLAST analyses. This BLAST analysis software samples domains found in the Smart and Pfam collections.

Consistent with other known members of the glycoprotein hormone beta subunit superfamily of proteins, human BRP contains a glycoprotein hormone beta chain domain and a cystine knot domain as shown in Table 2.

TABLE 2

| PSSMs producing significant alignments | Score(bits) | Evalue |
|---|---|---|
| gnl\|Smart\|smart00068 GHB, Glycoprotein | 73.6 | 6e−15 |
| hormone beta chain homologues | | |
| gnl\|Pfam\|pfam00007 Cys_knot, Cystine-knot | 58.2 | 3e−10 |
| domain | | |

The “E-value” or “Expect” value is a numeric indication of the probability that the aligned sequences could have achieved their similarity to the query sequence by chance alone, within the database that was searched. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences. The Expect value is used as a convenient way to create a significance threshold for reporting results. The default value used for blasting is typically set to 0.0001. The Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/.

An alignment of human BRP with the glycoprotein hormone beta chain domain consensus sequence as well as other members of the glycoprotein hormone beta superfamily of proteins is shown in Table 3. Black outlined amino acid residues indicate regions of identity; greyed amino acid residues indicate regions of conservative amino acid substitutions.

[image]

An alignment of human BRP with the glycoprotein hormone beta chain domain consensus sequence as well as other members of the cystine knot superfamily of proteins is shown in Table 4.

[image]

[image]

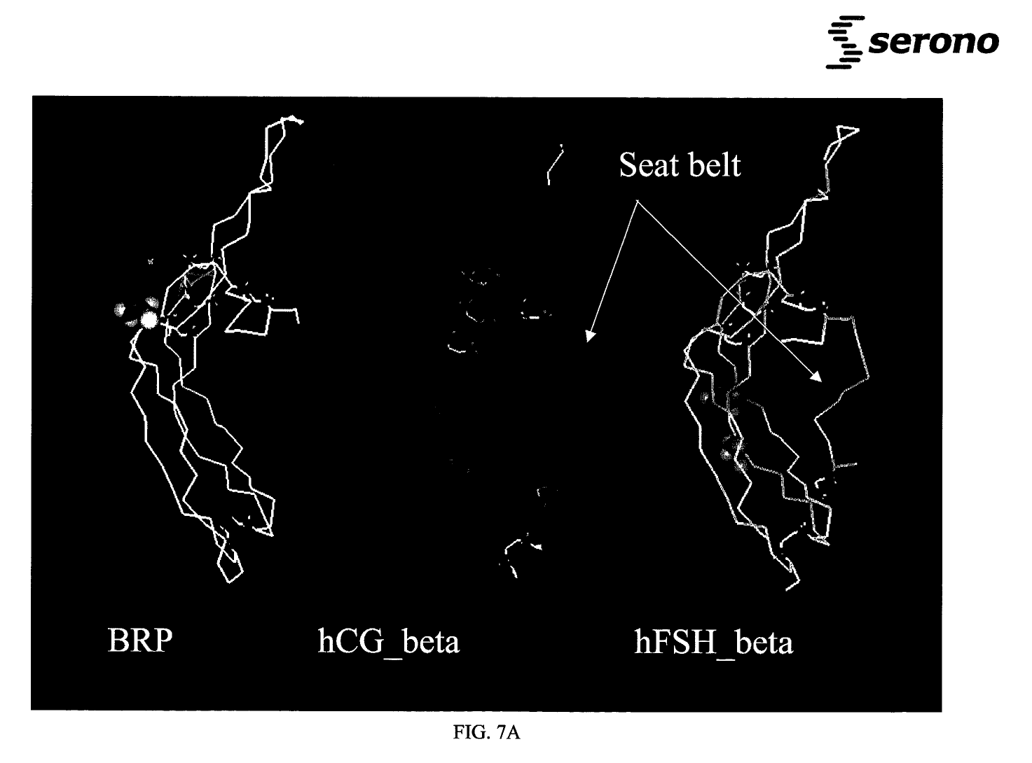

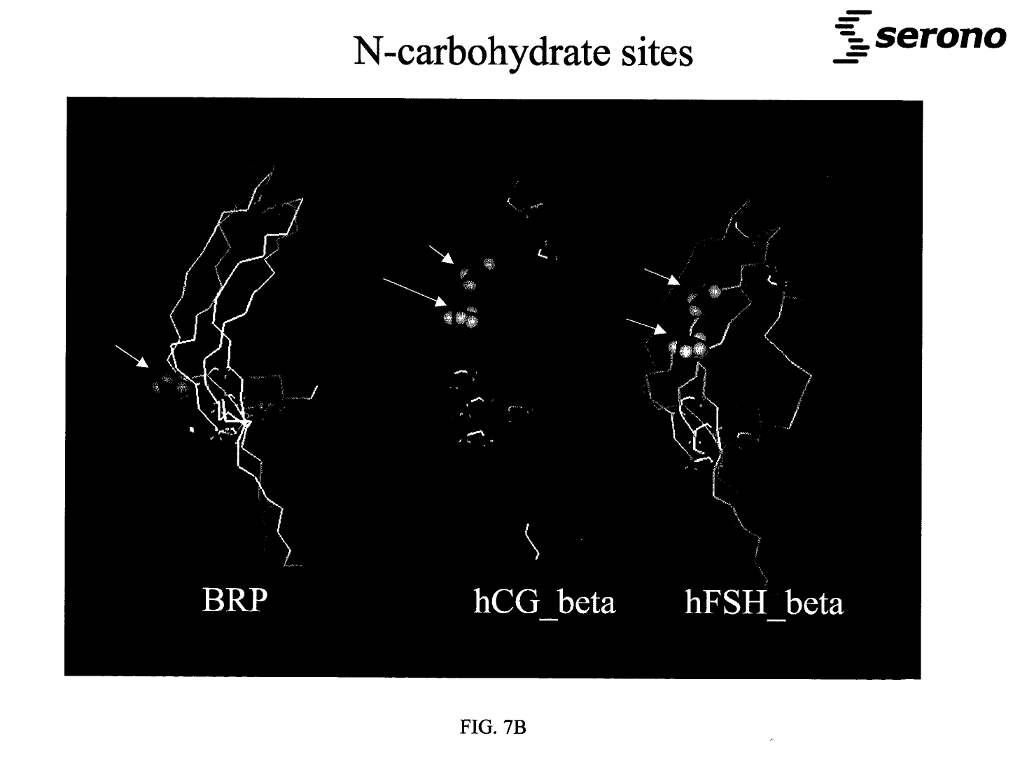

The putative signal peptide and the cysteine pattern of human BRP is similar to that of previously reported glycoprotein hormone subunits except for the absence of the seat-belt cysteines corresponding to cys residues 26 and 110 of choriogonadotropin beta subunit. (see FIG. 7A). In addition, the glycosylation pattern of the human BRP protein is different from that of known glycoprotein hormone beta subunits. (see FIG. 7B).

Multi-tissue expression (MTE) analysis identified BRPexpression in the pituitary.

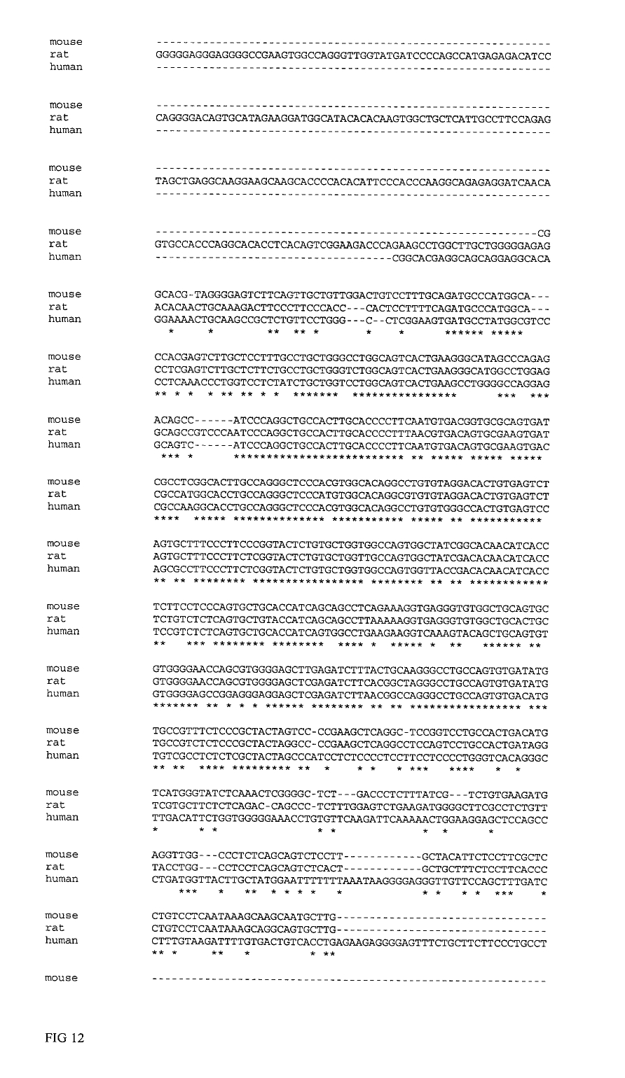

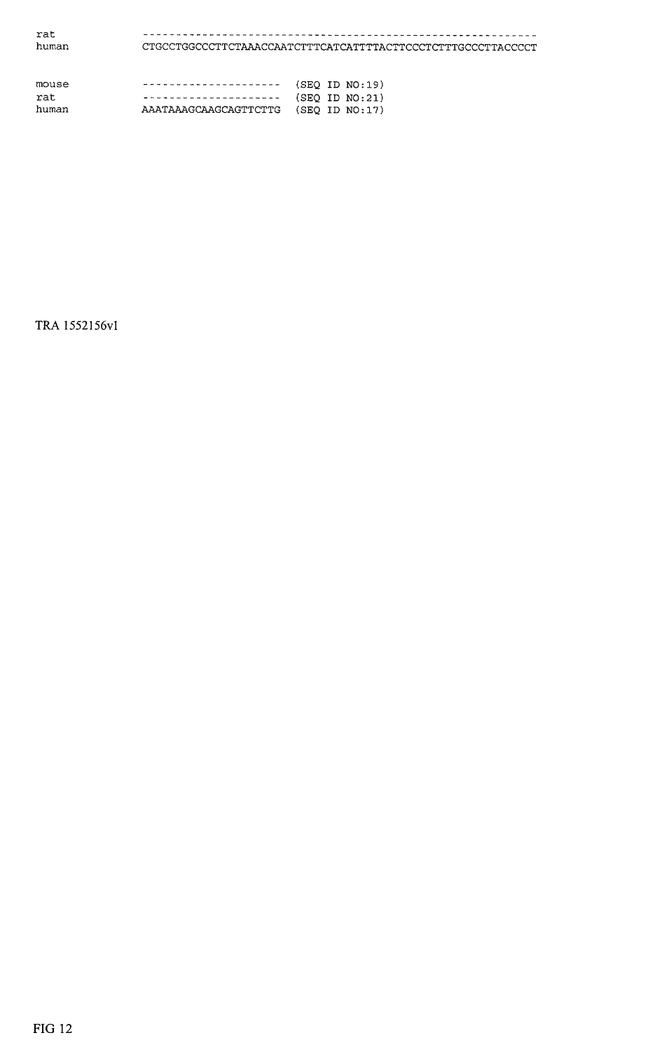

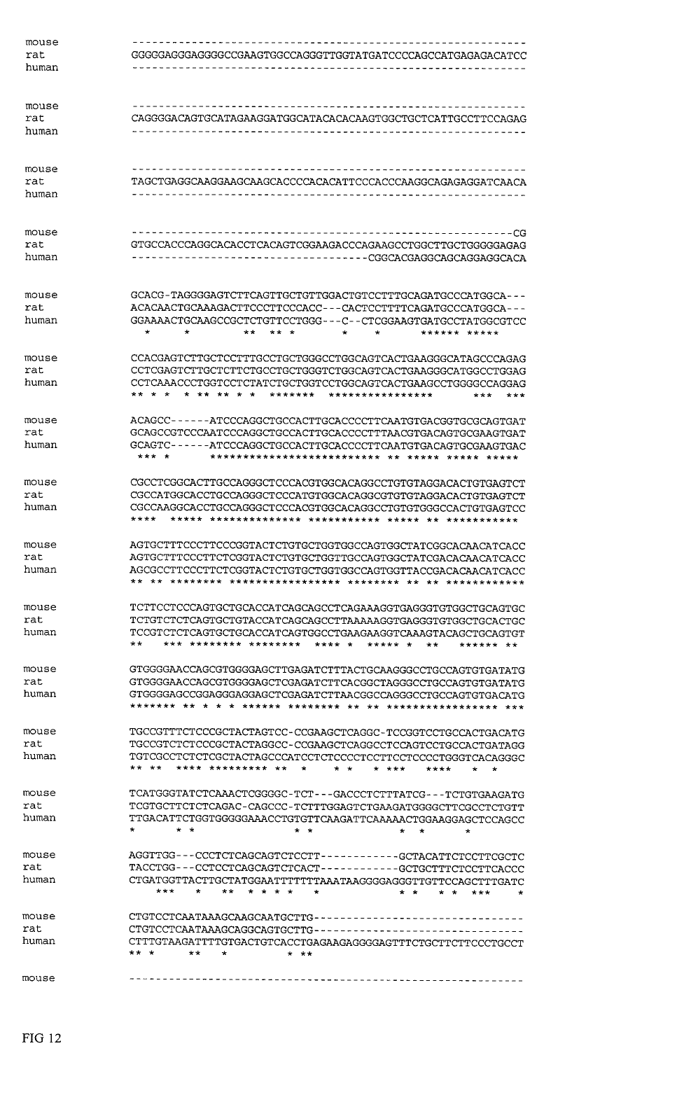

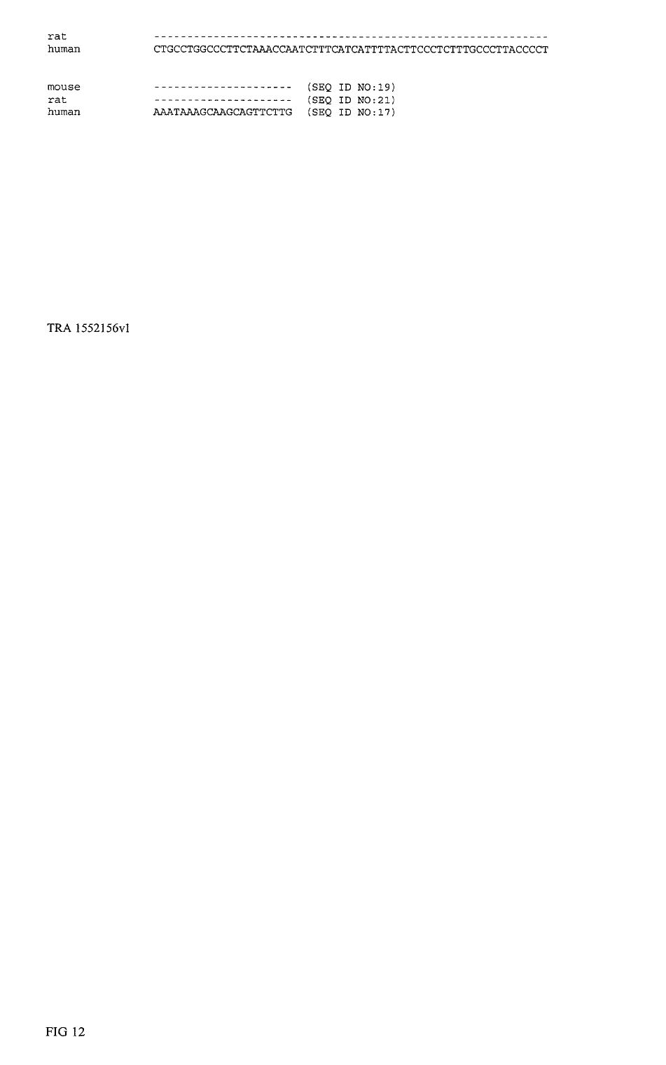

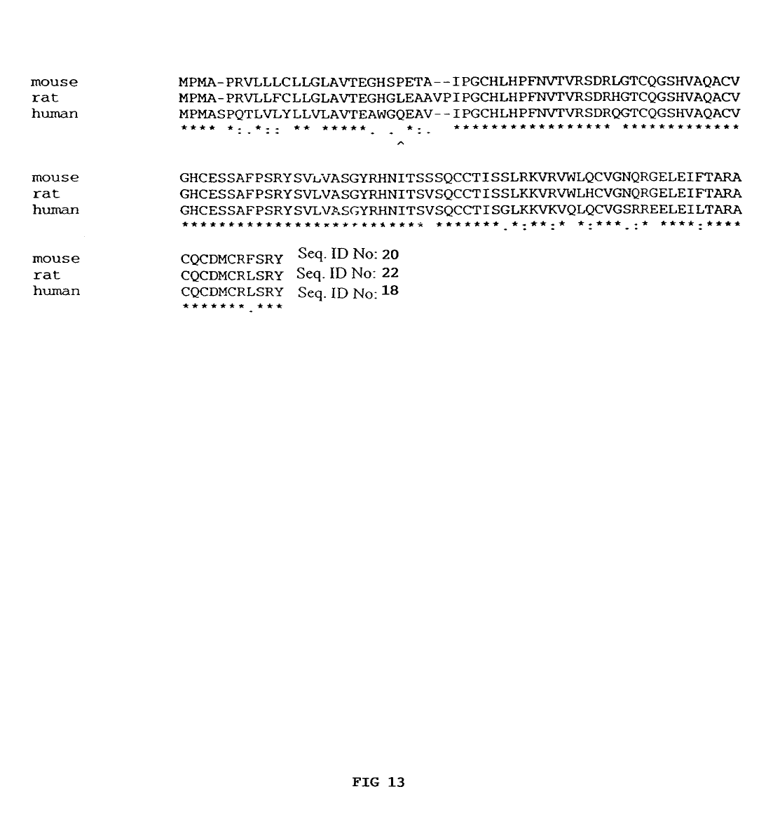

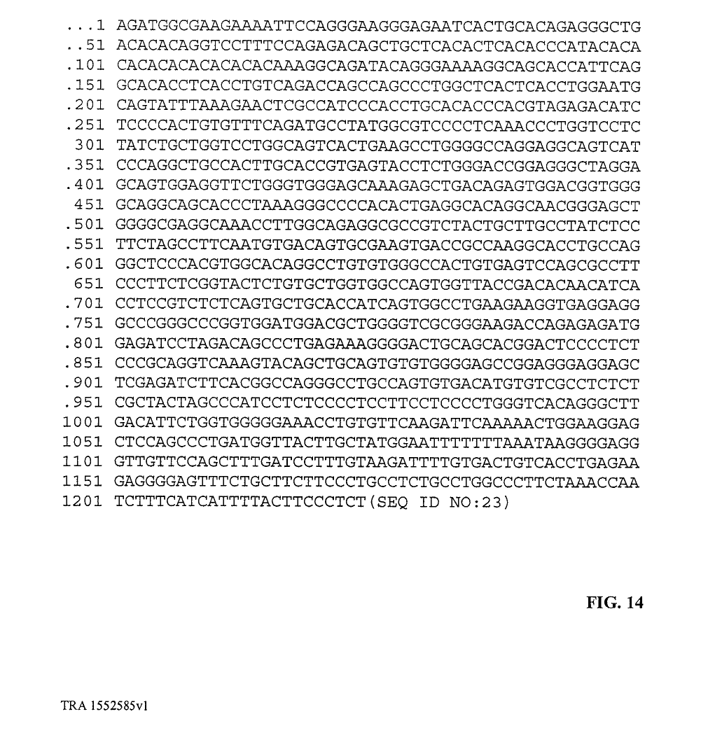

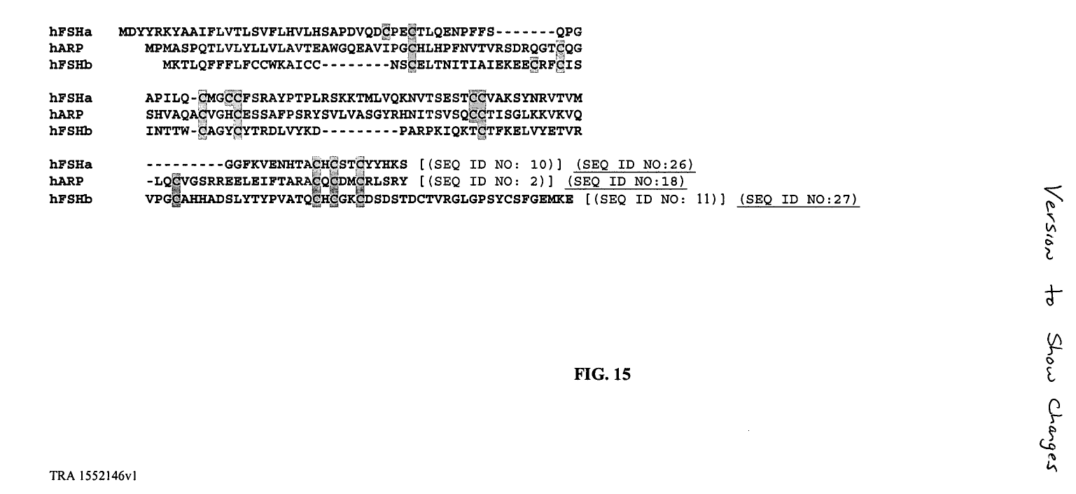

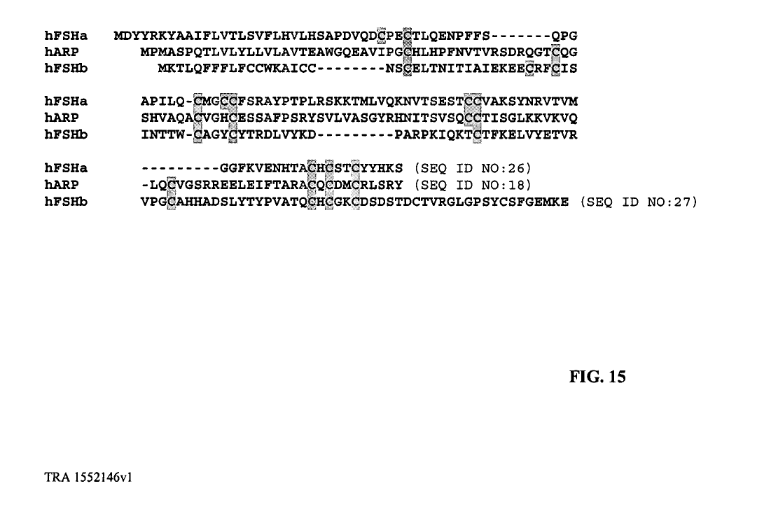

Also included in the invention are nucleotide sequences encoding a novel glycoprotein alpha subunit. (see FIG. 12; SEQ ID NO: 17, 19, and 21). GCG Spscan analysis predicted signal sequences in ARP. (SEQ ID NO: 28, 29 and 30). The ARP amino acid sequences of the encoded polypeptides are shown in FIG. 13 (SEQ ID NO: 18, 20 and 22).

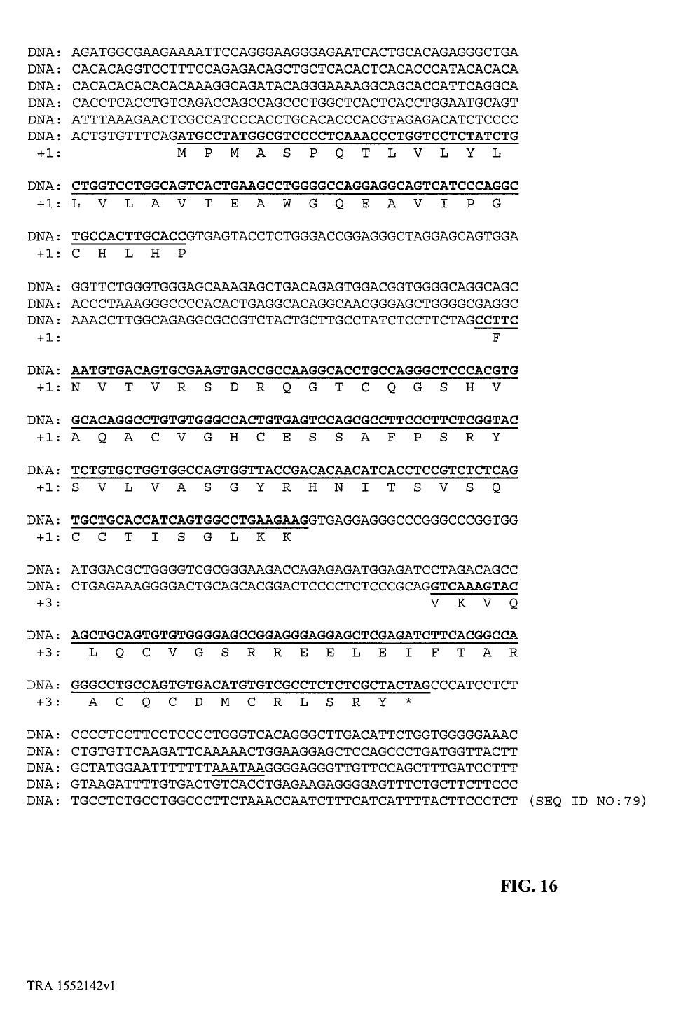

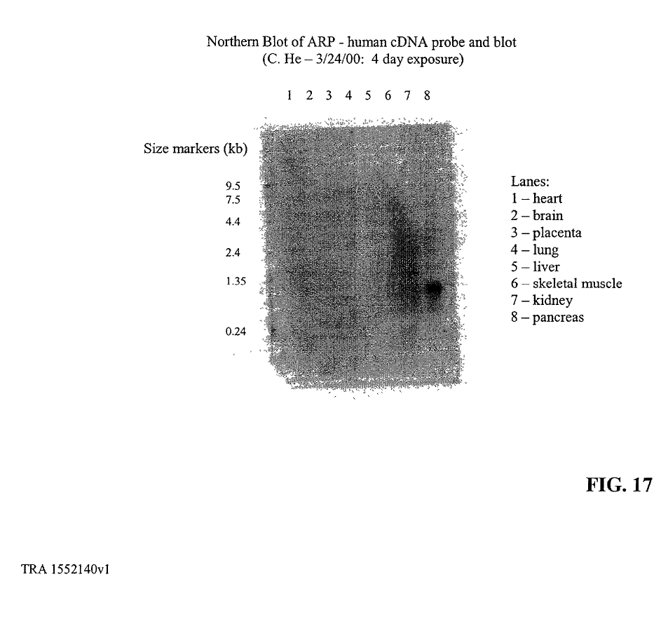

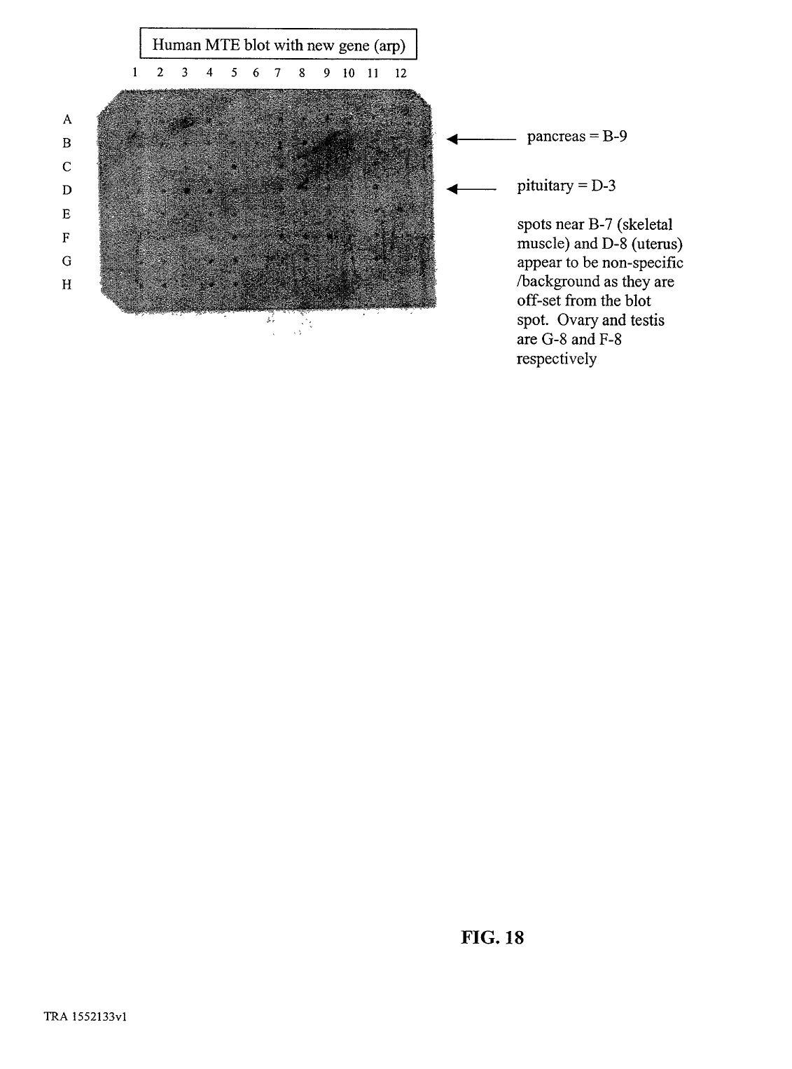

The ARP coding sequence is present in a BAC containing genomic DNA sequence from chromosome 11. (GenBank Accession No. AC000159). A full-length ARP coding region, containing a translational start site and termination codon was identified in the BAC. Northern analyis of ARP identifies a single mRNA species about 800-900 bases. (FIG. 17) The ARP coding region includes three exons and two introns in positions similar to the second and third intron positions in the known alpha subunit genes. (see FIG. 16) The ARP DNA sequence has 387 bases that encode a polypeptide predicted to have 129 amino acids (SEQ ID NO: 18).

The ARP nucleic acid sequence shows 21% identity to the alpha subunit and 14% idenity to the beta subunit of the glycoprotein family of hormones. The predicted mature coding region of the ARP protein shows 22% identity to the alpha subunit and 13% idenity to the beta subunit of the glycoprotein family of hormones.

Additionally, the peptide shares secondary structural motifs unique to each hormone unit. Similar to other alpha subunit proteins, ARP has an N-linked glycosylation site at Asn81 (counting from the initiation methionine) in loop 2. This glycosylation site and position is conserved in all alpha subunits and has been shown for several hormones to be critical for full hormone activity. Loop 2 is similar in length to the loop seen in the alpha subunits of the gonadatrophins and TSH. The ends of the loop sequence of ARP are consisitant with the alpha sequences of gonadotrophins and TSH. These end regions in alpha are known to be contact sites with the beta subunit, and across the contact sites in loops 2 and 3. A second N-linked glycosylation site is also present in loop 3 of the ARP protein in a position analogous to a site in the beta subunit.

ARP also has a cysteine pair near the middle of its sequence. The sequence corresponds to cysteines 59 & 60 in the alpha.subunit This feature is not found in beta subunits, however it is seen in other cystine knot proteins including members of the PDGF/VEGF family. The presence of the cysteine pair in the new sequence suggests an alpha-like disulfide bond between amino acid 59 and the cysteine corresponding to alpha amino acid 87.

Multi-tissue expression (MTE) analysis identified ARP expression in the pancreas and the pituitary.

Also included in the invention are ARP and BRP protein multimers (i.e., polymers). As used herein “multimer” and “polymer” are used interchangeably. For example, a multimer is a dimer. The ARP and BRP polypeptides or a fragment thereof, of the invention may form a multimer for example, with other apha or beta glycoprotein subunits to produce a functional glycoprotein hormone with similar, altered or enhanced activity to that of other related glycoprotein hormones (e.g., luteinizing hormone (LH), follicle stimulating hormone (FSH), thyroid stimulating hormone (TSH) and chorionic gonadotropin (hCG)). The multimer may be a homopolymer (i.e., ARP-ARP, BRP- BRP,) or alternatively a heteropolymer with a second polypeptide. The second polypeptide can be from the same species of ARP or BRP, e.g.,. Alternatively, the second polypeptide can be from a different species. Preferably, the second polypeptide is human. The second peptide may be a glycoprotein hormone beta or alpha subunit. Alternatively, the second peptide is a cystine knot protein, e.g., NGF, HCG, PDGF and TGF-beta2. For example, a BRP heteropolymer includes a BRP protein and an alpha glycoprotein subunit or a fragment thereof. Examples of an alpha glycoprotein subunit include, GenBank Acession Numbers, AAH10957, and CAC43234. Preferably, the BRP polypeptide forms a multimer with an ARP polypeptide. Alternatively, an ARP heteropolymer includes an ARP polypeptide and a beta glycoprotein subunit. Examples of an beta glycoprotein subunit include, GenBank Acession Numbers, P01225 and P18842. Preferably, the ARP polypeptide forms a multimer with an BRP polypeptide.

The similarity of ARP and BRP polypeptides to these previously described glycoproteins demonstrates that the ARP and BRP nucleic acids, polypeptides, protein multimers, antibodies and related compounds of the invention may be used to treat, prevent or diagnose a variety of reproductive and cell proliferative disorders. These disorders include for example, ovulatory disorders (i.e., stimulating follicular development and triggering ovulation), fertility related disorders, hypothyroidism, or metabolic disorders effecting pituitary function or pituitary target organs, e.g., adrenal gland, thyroid, gonad and liver. In addition, the BRP and ARP nucleic acid, polypeptides and protein multimers can be used to stimulate spermatogenesis, increase the function of the thyroid glandular cells (i.e., increase thyroid hormone production and iodide trapping), regulate gonadal function, regulate gonadal hormone production and promote or suppress fertility. The BRP and ARP nucleic acids and polypeptides can also be used to identify novel agents that modulate these disorders.

ARP/BRP Nucleic Acids

One aspect of the invention pertains to isolated nucleic acid molecules that encode ARP/BRP proteins or biologically active portions thereof, as well as nucleic acid fragments sufficient for use as hybridization probes to identify ARP/BRP-encoding nucleic acids (e.g., ARP/BRP mRNA) and fragments for use as PCR primers for the amplification or mutation of ARP/BRP nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

Also included in the invention are DNA constructs capable of modifying the expression of an endogenous ARP/BRP genomic sequences within the cell. Such constructs include a DNA regulatory sequence and a DNA targeting sequence. The DNA targeting sequence is capable of undergoing homogous recombination with a genomic sequence in the cell, thus placing the DNA regulatory connection to the operative conection to the endogenous ARP/BRP genomic sequence.

Further included in the invention are DNA constructs capable of amplifying the expression of an endogenous ARP/BRP genomic sequences within the cell. Such constructs include an amplifiable gene and a DNA targeting sequence. The DNA targeting sequence is capable of undergoing homogous recombination with a genomic sequence in the cell, thus placing the amplifiable gene to the operative connection to the endogenous ARP/BRP genomic sequence such that the ARP/BRP genomic sequence can be amplified

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated ARP/ BRP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., testis, or pituitary gland). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, 3, 17, 19, and 21 or a complement of any of these nucleotide sequences, can be isolated using standard molecular biology techniques and the sequence information provided herein. Using all or a portion of the nucleic acid sequences of SEQ ID NO: 1, 3, 17, 19, and 21 as a hybridization probe, ARP/BRP molecules can be isolated using standard hybridization and cloning techniques (e.g., as described in Sambrook et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to ARP/BRP nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO: 1, 3, 17, 19, and 21, or a complement thereof. Oligonucleotides may be chemically synthesized and may be used as probes.

In another embodiment, an isolated nucleic acid molecule of the invention comprises a nucleic acid molecule that is a complement of the nucleotide sequence shown in SEQ ID NO: 1, 3, 17, 19, and 21. In another embodiment, an isolated nucleic acid molecule of the invention comprises a nucleic acid molecule that is a complement of the nucleotide sequence shown in SEQ ID NO: 1, 3, 17, 19, and 21, or a portion of this nucleotide sequence. A nucleic acid molecule that is complementary to the nucleotide sequence shown in SEQ ID NO: 1, 3, 17, 19, and 21 is one that is sufficiently complementary to the nucleotide sequence shown in SEQ ID NO: 1, 3, 17, 19, and 21 that it can hydrogen bond with little or no mismatches to the nucleotide sequence shown in SEQ ID NO: 1, 3, 17, 19, and 21, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Van der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

In one aspect, the isolated nucleic acid molecule of the invention, e.g., a BRP nucleic acid, comprises contiguous nucleotides encoding the amino acid sequence WEKPI (SEQ ID NO: 5).

Alternativley, the isolated nucleic acid molecule of the invention, e.g., an ARP nucleic acid, comprises contiguous nucleotides encoding the amino acid sequence LHPFNV (SEQ ID NO: 24), In an alternative emodiment the isolated nucleic acid molecule of the invention e.g., an ARP nucleic acid, comprises contiguous nucleotides encoding the amino acid sequence LKKVKV (SEQ ID NO: 25). Optimally, the isolated nucleic acid molecule of the invention comprises contiguous nucleotides encoding the amino acid sequence LHPFNV (SEQ ID NO: 24) and LKKVKV (SEQ ID NO: 25).

Moreover, the nucleic acid molecule of the invention can comprise only a portion of the nucleic acid sequence of SEQ ID NO: 1, 3, 17, 19 or 21, e.g., a fragment that can be used as a probe or primer or a fragment encoding a biologically active portion of ARP/BRP.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of ARP/BRP polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a ARP/BRP polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding ARP/BRP protein. Homologous nucleic acid sequences include those nucleic acid sequences that encode conservative amino acid substitutions (see below) in. SEQ ID NO: 1, 3, 17, 19, and 21, as well as a polypeptide having ARP/BRP activity. Biological activities of the ARP/BRP proteins are described below.

An ARP/BRP polypeptide is encoded by the open reading frame (“ORF”) of a ARP/BRP nucleic acid. The invention includes the nucleic acid sequence comprising the stretch of nucleic acid sequences of SEQ ID NO: 3, that comprises the ORF of that nucleic acid sequence and encodes a polypeptide of SEQ ID NO: 4.

An “open reading frame” (“ORF”) corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, for example, a stretch of DNA that would encode a protein of 50 amino acids or more.

The nucleotide sequence determined from the cloning of the ARP/BRP gene allows for the generation of probes and primers designed for use in identifying and/or cloning ARP/BRP homologues in other cell types, e.g. from other tissues, as well as ARP/BRP homologues from other mammals. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 1, 3, 17, 19, and 21, or an anti-sense strand nucleotide sequence of SEQ ID NO: 1, 3, 17, 19, and 21 or of a naturally occurring mutant of SEQ ID NO: 1, 3, 17, 19, and 21.

Probes based on the ARP/BRP nucleotide sequence can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissue which misexpress a ARP/BRP protein, such as by measuring a level of a ARP/BRP-encoding nucleic acid in a sample of cells from a subject e.g., detecting ARP/BRP mRNA levels or determining whether a genomic ARP/BRP gene has been mutated or deleted.

“A polypeptide having a biologically active portion of ARP/BRP” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of ARP/BRP” can be prepared by isolating a portion of SEQ ID NO: 1, 3, 17, 19, and 21 that encodes a polypeptide having a ARP/BRP biological activity (the biological activities of the ARP/BRP proteins are described below), expressing the encoded portion of ARP/BRP protein (e.g., by recombinant expression in vitro) and assessing the activity of the encoded portion of ARP/BRP.

ARP/BRP Variants

The invention further encompasses nucleic acid molecules that differ from the nucleotide sequence shown in SEQ ID NO: 1, 3, 17, 19, and 21 due to degeneracy of the genetic code and thus encode the same ARP/BRP protein as that encoded by the nucleotide sequence shown in SEQ ID NO: 1, 3, 17, 19, and 21. In another embodiment, an isolated nucleic acid molecule of the invention has a nucleotide sequence encoding a protein having an amino acid sequence shown in SEQ ID NO: 2, 4, 18, 20, and 22.

In addition to the ARP/BRP nucleotide sequence shown in SEQ ID NO: 1, 3, 17, 19, and 21 it will be appreciated by those skilled in the art that DNA sequence polymorphisms that lead to changes in the amino acid sequences may exist within a population (e.g., the population). Such genetic polymorphism in the ARP/BRP gene may exist among individuals within a population due to natural allelic variation. As used herein, the terms “gene” and “recombinant gene” refer to nucleic acid molecules comprising an open reading frame encoding a ARP/BRP protein, preferably a mammalian ARP/BRP protein. Such natural allelic variations can typically result in 1-5% variance in the nucleotide sequence of the ARP/BRP gene. Any and all such nucleotide variations and resulting amino acid polymorphisms in ARP/BRP that are the result of natural allelic variation and that do not alter the functional activity of ARP/BRP are intended to be within the scope of the invention.

Moreover, nucleic acid molecules encoding ARP/BRP proteins from other species, and thus that have a nucleotide sequence that differs from the sequence of SEQ ID NO: 1, 3, 17, 19, and 21 are intended to be within the scope of the invention. Nucleic acid molecules corresponding to natural allelic variants and homologues of the ARP/BRP cDNAs of the invention can be isolated based on their homology to the ARP/BRP nucleic acids disclosed herein using the cDNAs, or a portion thereof, as a hybridization probe according to standard hybridization techniques under stringent hybridization conditions. For example, a soluble ARP/BRP cDNA can be isolated based on its homology to membrane-bound ARP/BRP. Likewise, a membrane-bound ARP/BRP cDNA can be isolated based on its homology to soluble ARP/BRP.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 3, 17, 19, and 21. In another embodiment, the nucleic acid is at least 10, 25, 50, 100, 250, 500, 750, 1000 or 1250 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i e., nucleic acids encoding ARP/BRP proteins derived from species other than) or other related sequences (e.g., paralogs) can be obtained by low, moderate or high stringency hybridization with all or a portion of the particular sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), CURRENT PROTOCOLS TN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequence of SEQ ID NO: 1, 3, 17, 19, and 21 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 3, 17, 19, and 21 , or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, 3, 17, 19, and 21, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 25 78: 6789-6792.

Conservative Mutations

In addition to naturally-occurring allelic variants of the ARP/BRP sequence that may exist in the population, the skilled artisan will further appreciate that changes can be introduced by mutation into the nucleotide sequence of SEQ ID NO: 1, 3, 17, 19, and 21, thereby leading to changes in the amino acid sequence of the encoded ARP/BRP protein, without altering the functional ability of the ARP/BRP protein. For example, nucleotide substitutions leading to amino acid substitutions at “non-essential” amino acid residues can be made in the sequence of SEQ ID NO: 1, 3, 17, 19, and 21. A “non-essential” amino acid residue is a residue that can be altered from the wild-type sequence of ARP/BRP without altering the biological activity, whereas an “essential” amino acid residue is required for biological activity. For example, amino acid residues that are conserved among the ARP/BRP proteins of the present invention, are predicted to be particularly unamenable to alteration.

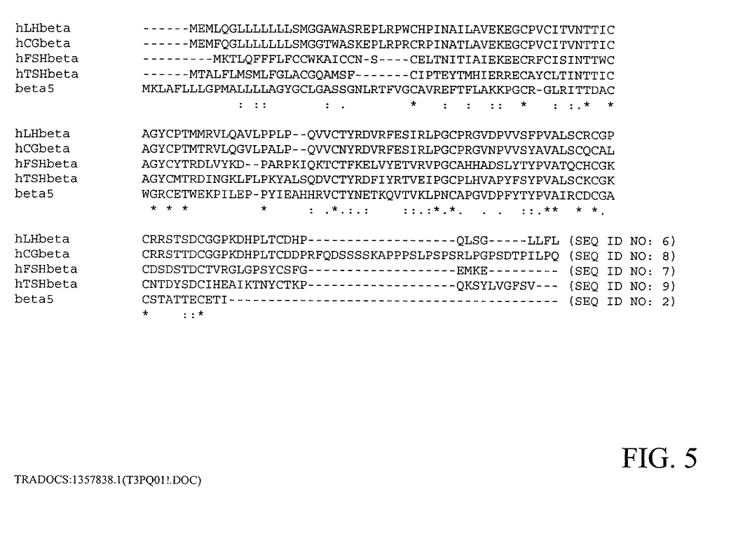

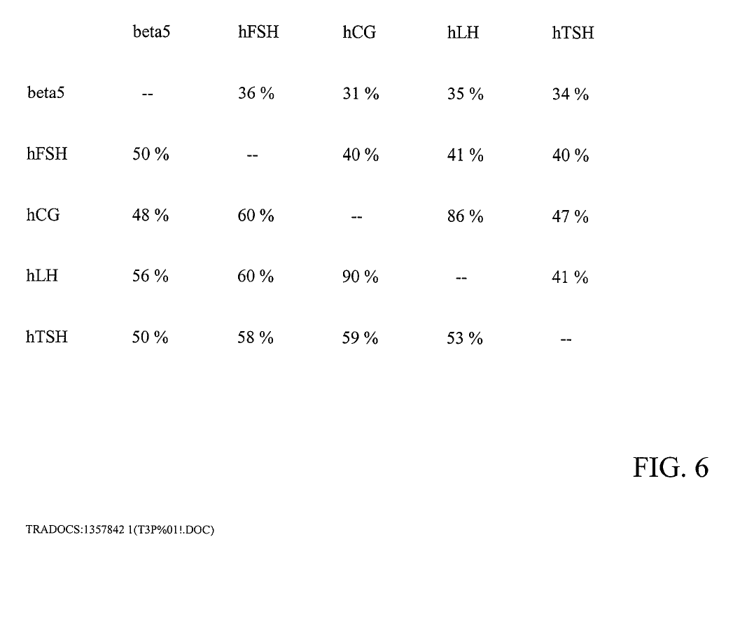

In addition, amino acid residues that are conserved among family members of the ARP/BRP proteins of the present invention, as indicated by the alignment presented as FIG. 5, are also predicted to be particularly unamenable to alteration. For example, ARP/BRP proteins of the present invention can contain at least one cystine knot domain that is a typically conserved region in ARP/BRP family members and ARP/BRP homologs. As such, these conserved domains are not likely to be amenable to mutation. Other amino acid residues, however, (e.g., those that are not conserved or only semi-conserved among members of the ARP/BRP proteins) may not be essential for activity and thus are likely to be amenable to alteration.

Another aspect of the invention pertains to nucleic acid molecules encoding ARP/BRP proteins that contain changes in amino acid residues that are not essential for activity. Such ARP/BRP proteins differ in amino acid sequence from SEQ ID NO: 2, 4, 18, 20, and 22, yet retain biological activity. In one embodiment, the isolated nucleic acid molecule comprises a nucleotide sequence encoding a protein, wherein the protein comprises an amino acid sequence at least about 45% homologous to the amino acid sequence of SEQ ID NO: 2, 4, 18, 20, and 22. Preferably, the protein encoded by the nucleic acid molecule is at least about 60% homologous to SEQ ID NO: 2, 4, 18, 20, and 22, more preferably at least about 70% homologous to SEQ ID NO: 2, 4, 18, 20, and 22, still more preferably at least about 80% homologous to SEQ ID NO: 2, 4, 18, 20, and 22, even more preferably at least about 90% homologous to SEQ ID NO: 2, 4, 18, 20, and 22, and most preferably at least about 95% homologous to SEQ ID NO: 2, 4, 18, 20, and 22.

An isolated nucleic acid molecule encoding a ARP/BRP protein homologous to the protein of SEQ ID NO: 2, 4, 18, 20, and 22 can be created by introducing one or more nucleotide substitutions, additions or deletions into the nucleotide sequence of SEQ ID NO: 1, 3, 17, 19, and 21 such that one or more amino acid substitutions, additions or deletions are introduced into the encoded protein.

Mutations can be introduced into SEQ ID NO: 1, 3, 17, 19, and 21 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in ARP/BRP is replaced with another amino acid residue from the same side chain family. Alternatively, in another embodiment, mutations can be introduced randomly along all or part of a ARP/BRP coding sequence, such as by saturation mutagenesis, and the resultant mutants can be screened for ARP/BRP biological activity to identify mutants that retain activity. Following mutagenesis of SEQ ID NO: 1, 3, 17, 19, and 21, the encoded protein can be expressed by any recombinant technology known in the art and the activity of the protein can be determined.

In one embodiment, a mutant ARP/BRP protein can be assayed for (1) the ability to form protein:protein interactions with other ARP/BRP proteins, other cell-surface proteins, or biologically active portions thereof, (2) complex formation between a mutant ARP/BRP protein and a ARP/BRP ligand; (3) the ability of a mutant ARP/BRP protein to bind to an intracellular target protein or biologically active portion thereof, (e.g. avidin proteins).

In yet another embodiment, a mutant ARP/BRP can be assayed for the ability to perform glycoprotein hormone family member activities, such as, complex formation i.e. binding, between (i) a ARP/BRP protein and a glycoprotein receptor; (ii) a protein having substantial homology to the cystine knot family of proteins; (iii) a ARP/BRP protein with a LGR orphan G-protein coupled receptor family member protein; and (iv) a ARP/BRP protein with a glycoprotein hormone.

Antisense

Another aspect of the invention pertains to isolated antisense nucleic acid molecules that are hybridizable to or complementary to the nucleic acid molecule comprising the nucleotide sequence of SEQ ID NO: 1, 3, 17, 19, and 21, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire ARP/BRP coding strand, or to only a portion thereof. Nucleic acid molecules encoding fragments, homologs, derivatives and analogs of a ARP/BRP protein of SEQ ID NO: 2, 4, 18, 20, and 22, or antisense nucleic acids complementary to a ARP/BRP nucleic acid sequence of SEQ ID NO: 1, 3, 17, 19, and 21, are additionally provided.

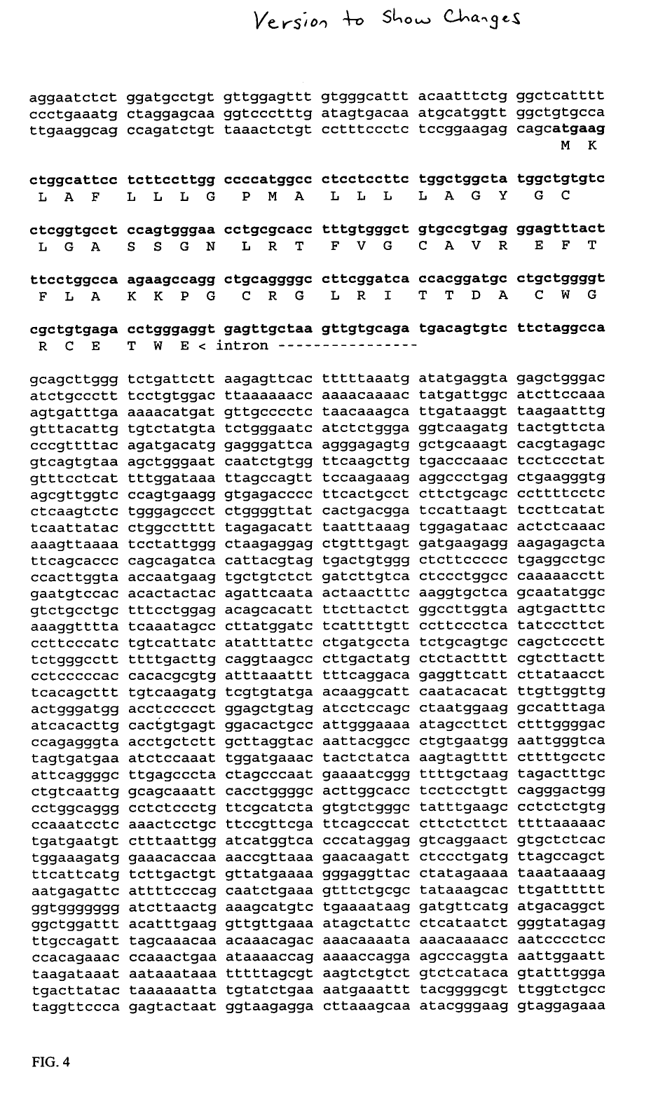

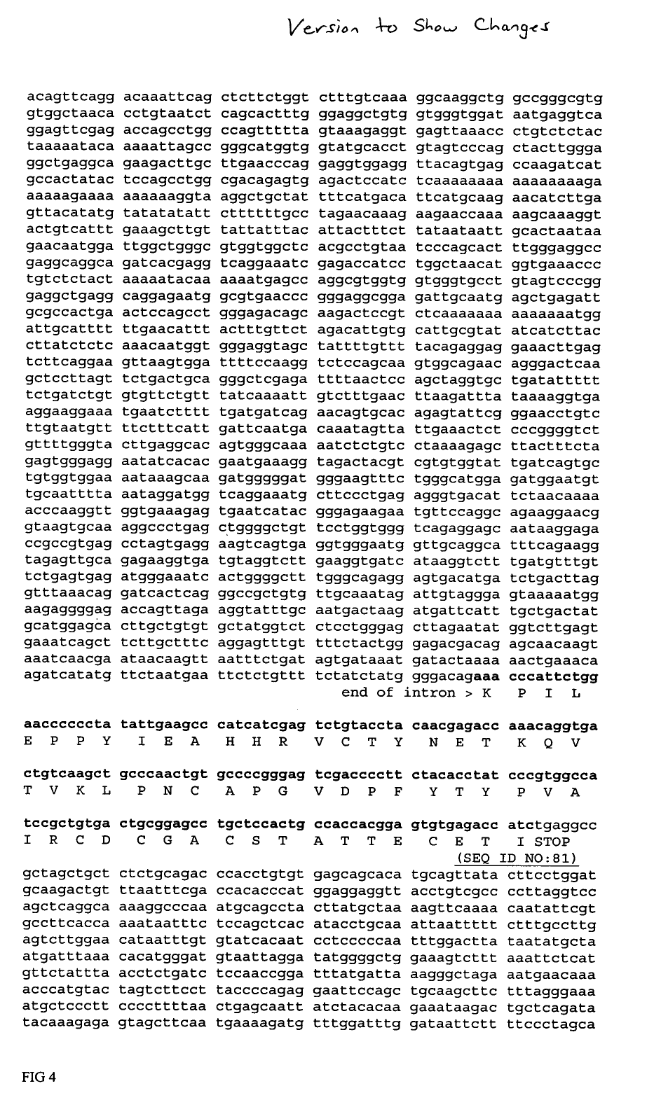

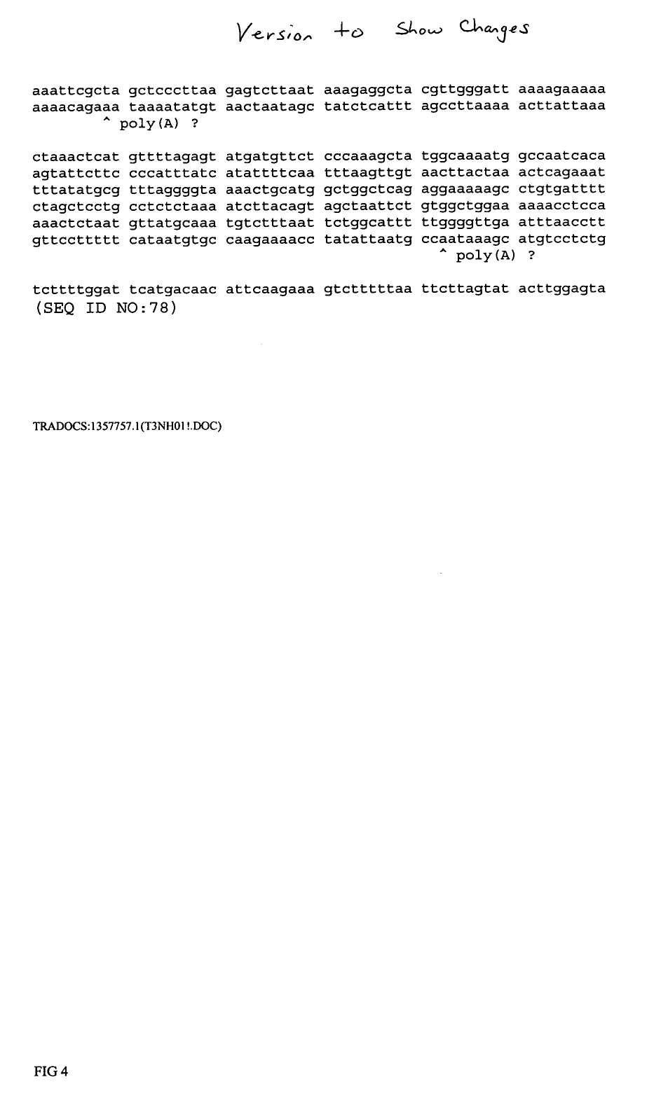

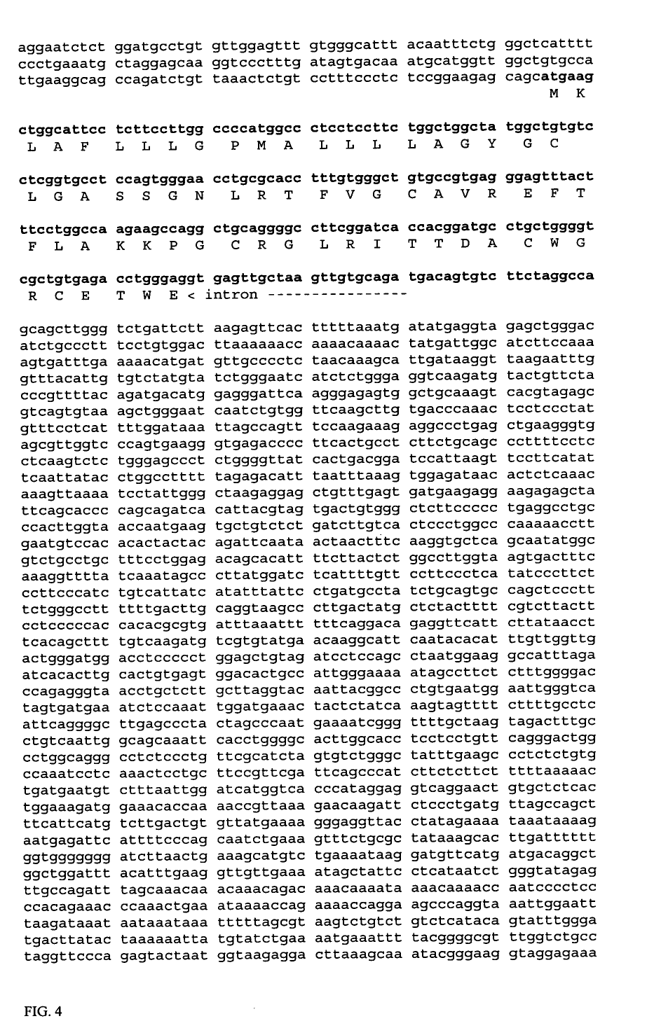

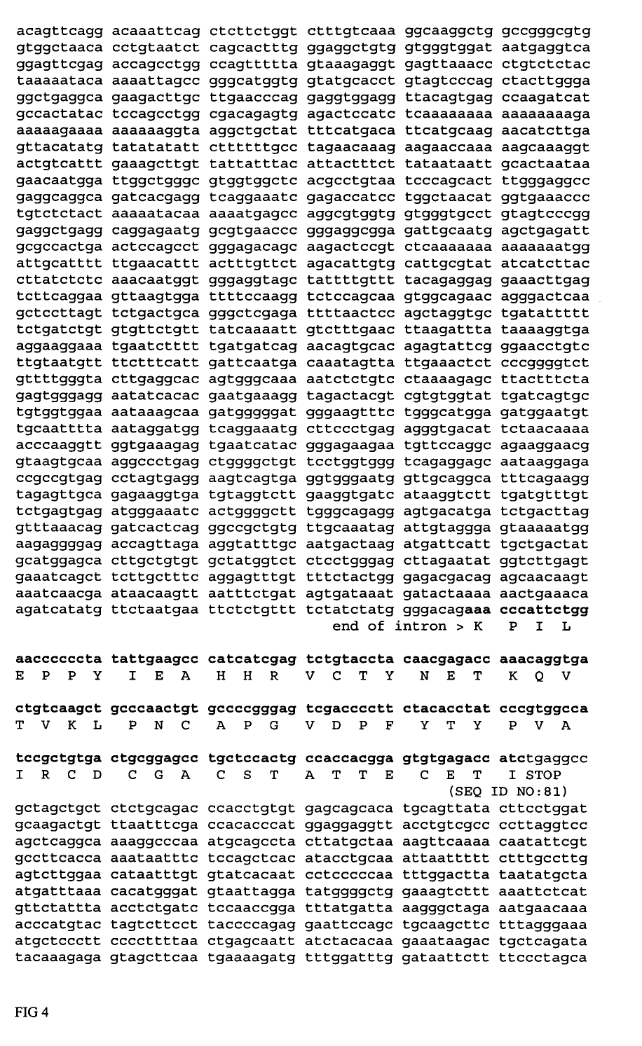

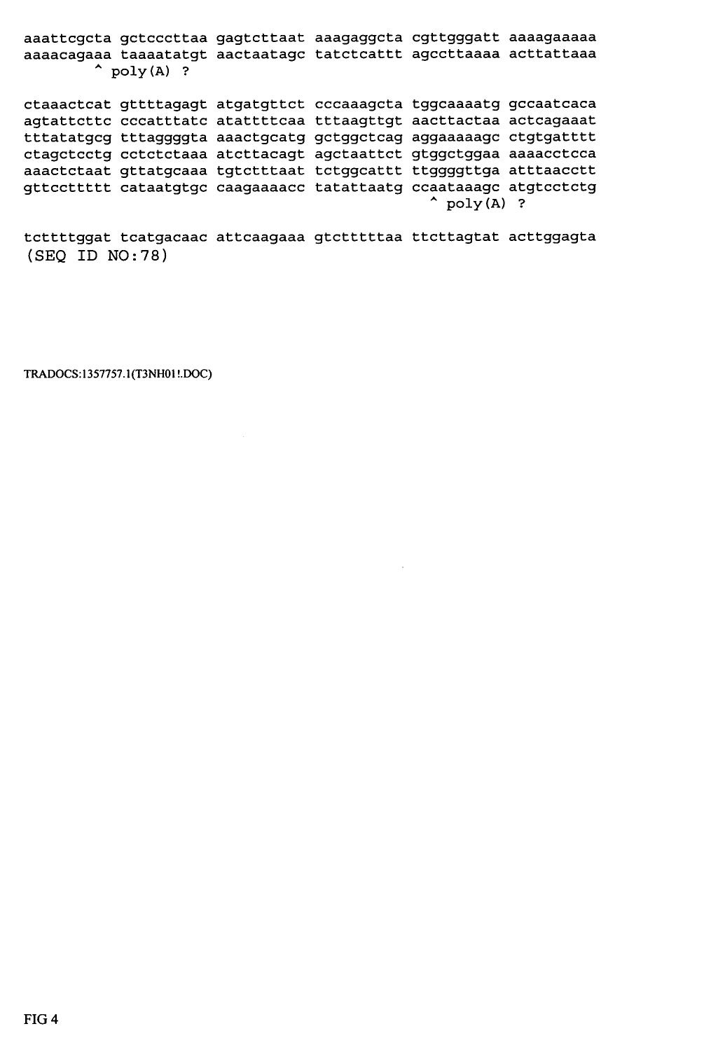

In one embodiment, an antisense nucleic acid molecule is antisense to a “coding region” of the coding strand of a nucleotide sequence encoding ARP/BRP. The term “coding region” refers to the region of the nucleotide sequence comprising codons which are translated into amino acid residues (see, e.g., FIG. 4). In another embodiment, the antisense nucleic acid molecule is antisense to a “noncoding region” of the coding strand of a nucleotide sequence encoding ARP/BRP. The term “noncoding region” refers to 5′ and 3′ sequences which flank the coding region that are not translated into amino acids (i.e., also referred to as 5′ and 3′ untranslated regions).

Given the coding strand sequences encoding ARP/BRP disclosed herein (e.g., SEQ ID NO: 1, 3, 17, 19, and 21 ), antisense nucleic acids of the invention can be designed according to the rules of Watson and Crick or Hoogsteen base pairing. The antisense nucleic acid molecule can be complementary to the entire coding region of ARP/BRP mRNA, but more preferably is an oligonucleotide that is antisense to only a portion of the coding or noncoding region of ARP/BRP mRNA. For example, the antisense oligonucleotide can be complementary to the region surrounding the translation start site of ARP/BRP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a ARP/BRP protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1 987) FEBS Lett 215: 327-330).

Ribozymes and PNA Moieties

Nucleic acid modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave ARP/BRP mRNA transcripts to thereby inhibit translation of ARP/BRP mRNA. A ribozyme having specificity for a ARP/BRP-encoding nucleic acid can be designed based upon the nucleotide sequence of a ARP/BRP cDNA disclosed herein (i.e., SEQ ID NO: 1, 3, 17, 19, and 21). For example, a derivative of a Tetrahymena L-19 IVS RNA can be constructed in which the nucleotide sequence of the active site is complementary to the nucleotide sequence to be cleaved in a ARP/BRP-encoding mRNA. See, e.g., Cech et al. U.S. Pat. No. 4,987,071; and Cech et al. U.S. Pat. No. 5,116,742. Alternatively, ARP/BRP mRNA can be used to select a catalytic RNA having a specific ribonuclease activity from a pool of RNA molecules. See, e.g., Bartel et al., (1993) Science 261:1411-1418.

Alternatively, ARP/BRP gene expression can be inhibited by targeting nucleotide sequences complementary to the regulatory region of the ARP/BRP (e.g., the ARP/BRP promoter and/or enhancers) to form triple helical structures that prevent transcription of the ARP/BRP gene in target cells. See generally, Helene. (1991) Anticancer Drug Des. 6: 569-84; Helene. et al. (1992) Ann. N.Y Acad. Sci 660:27-36; and Maher (1992) Bioassays 14: 807-15.

In various embodiments, the nucleic acids of ARP/BRP can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of ARP/BRP can be used in therapeutic and diagnostic applications. For example, PNAs can be used as antisense or antigene agents for sequence-specific modulation of gene expression by, e.g., inducing transcription or translation arrest or inhibiting replication. PNAs of ARP/BRP can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of ARP/BRP can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of ARP/BRP can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA SEQment and a 3′ DNA SEQment (Finn et al. (1996) above). Alternatively, chimeric molecules can be synthesized with a 5′ DNA SEQment and a 3′ PNA SEQment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

Nucleotide Polymorphisms Associated with ARP/BRP Genes

The invention also includes nucleic acid sequences that include one or more polymorphic ARP/BRP sequences. Also included are methods of identifying a base occupying a polymorphic in an ARP/BRP sequence, as well as methods of identifying an individualized therapeutic agent for treating ARP/BRP associated pathologies based on ARP/BRP sequence polymorphisms.

The nucleotide polymorphism can be a single nucleotide polymorphism (SNP). A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

For example, a polymorphism according to the invention includes a sequence polymorphism in the ARP gene in which the adenosine at nucleotide 342 is replaced by cytosine. (Fog. 27) This results in a amino acid change of a Leu to a Phe in the ARP polypeptide sequence at position 114. In some embodiments the polymorphic sequence includes a nucleotide sequence of an ARP gene, wherein the nucleotide at 342 is any nucleotide other that adenosine.

In some embodiments, the polymorphic sequence includes the full length of any ARP/BRP. In other embodiments, the polymorphic sequence includes a polynucleotide that is between 10 and 100 nucleotides, 10 and 75 nucleotides, 10 and 50 nucleotides, or 10 and 25 nucleotides in length.

ARP/BRP Proteins

One aspect of the invention pertains to isolated ARP/BRP proteins, and biologically active portions thereof, or derivatives, fragments, analogs or homologs thereof. Also provided are polypeptide fragments suitable for use as immunogens to raise anti-ARP/BRP antibodies. In one embodiment, native ARP/BRP proteins can be isolated from cells or tissue sources by an appropriate purification scheme using standard protein purification techniques. In another embodiment, ARP/BRP proteins are produced by recombinant DNA techniques. Alternative to recombinant expression, a ARP/BRP protein or polypeptide can be synthesized chemically using standard peptide synthesis techniques. The ARP/BRP proteins may be glycosylated at one or more sites. Alternatively, the ARP/BRP protein is not glycosylated

An “isolated” or “purified” protein or biologically active portion thereof is substantially free of cellular material or other contaminating proteins from the cell or tissue source from which the ARP/BRP protein is derived, or substantially free from chemical precursors or other chemicals when chemically synthesized. The language “substantially free of cellular material” includes preparations of ARP/BRP protein in which the protein is separated from cellular components of the cells from which it is isolated or recombinantly produced. In one embodiment, the language “substantially free of cellular material” includes preparations of ARP/BRP protein having less than about 30% (by dry weight) of non-ARP/BRP protein (also referred to herein as a “contaminating protein”), more preferably less than about 20% of non-ARP/BRP protein, still more preferably less than about 10% of non-ARP/BRP protein, and most preferably less than about 5% non-ARP/BRP protein. When the ARP/BRP protein or biologically active portion thereof is recombinantly produced, it is also preferably substantially free of culture medium, i e., culture medium represents less than about 20%, more preferably less than about 10%, and most preferably less than about 5% of the volume of the protein preparation.

The language “substantially free of chemical precursors or other chemicals” includes preparations of ARP/BRP protein in which the protein is separated from chemical precursors or other chemicals that are involved in the synthesis of the protein. In one embodiment, the language “substantially free of chemical precursors or other chemicals” includes preparations of ARP/BRP protein having less than about 30% (by dry weight) of chemical precursors or non-ARP/BRP chemicals, more preferably less than about 20% chemical precursors or non-ARP/BRP chemicals, still more preferably less than about 10% chemical precursors or non-ARP/BRP chemicals, and most preferably less than about 5% chemical precursors or non-ARP/BRP chemicals.

Biologically active portions of a ARP/BRP protein include peptides comprising amino acid sequences sufficiently homologous to or derived from the amino acid sequence of the ARP/BRP protein, e.g., the amino acid sequence shown in SEQ ID NO: 2, 4, 18, 20, and 22, that include fewer amino acids than the full length ARP/BRP proteins, and exhibit at least one activity of a ARP/BRP protein. Typically, biologically active portions comprise a domain or motif with at least one activity of the ARP/BRP protein. A biologically active portion of a ARP/BRP protein can be a polypeptide which is, for example, 10, 25, 50, 100 or more amino acids in length.

In one embodiment, a biologically active portion of a ARP/BRP protein comprises at least one cystine knot domain characteristic of the glycoprotein family of proteins.

In yet another embodiment, a biologically active portion of a ARP protein comprises at least one N-linked-glycosylation site in loop 2, characteristic of the glycoprotein hormone family of proteins, optimally the alpha subunit of the hormone.

It is to be understood that a biologically active portion of a ARP/BRP protein of the present invention may contain at least one of the above-identified structural domains. Moreover, other biologically active portions, in which other regions of the protein are deleted, can be prepared by recombinant techniques and evaluated for one or more of the functional activities of a native ARP/BRP protein.

In an embodiment, the ARP/BRP protein has an amino acid sequence shown in SEQ ID NO: 2, 4, 18, 20, and 22. In other embodiments, the ARP/BRP protein is substantially homologous to SEQ ID NO: 2, 4, 18, 20, and 22 and retains the functional activity of the protein of SEQ ID NO: 2, 4, 18, 20, and 22 yet differs in amino acid sequence due to natural allelic variation or mutagenesis, as described in detail below. Accordingly, in another embodiment, the ARP/BRP protein is a protein that comprises an amino acid sequence at least about 45% homologous to the amino acid sequence of SEQ ID NO: 2, 4, 18, 20, and 22 and retains the functional activity of the ARP/BRP proteins of SEQ ID NO: 2, 4, 18, 20, and 22.

In another embodiment, the ARP/BRP protein is a protein having an amino acid sequence 55% homologous to a cystine knot domain of SEQ ID NO: 2 (e.g., about amino acid residues 30-129, or amino acid residues 21-124). Another embodiment of the invention features isolated ARP/BRP protein having and amino acid sequence at least about 65%, preferably 75%, 85%, or 95% homologous to a cystine knot domain of SEQ ID NO: 2, 4, 18, 20, and 22 (e.g., about amino acid residues 31-124). In one embodiment, the ARP/BRP protein retains the functional activity of the ARP/BRP proteins of SEQ ID NO: 2, 4, 18, 20, and 22.

ARP/BRP Multimers

Also provided by the the invention are ARP and BRP protein multimers (i.e., polymer). A multimer is for example a dimer, trimer, or tetramer. A multimer comprises a ARP or BRP protein, or a biologically active portions thereof, or derivatives, fragments, analogs or homologs thereof and a second polypeptide. The polypeptides of the multimer interact covalently, e.g., disulfide bond, or non-covalently. Alternatively, the polypeptides of the multimer may be chemically linked.

The ARP and ARP/BRP polypeptides or a fragment thereof, of the invention may form a multimer for example, with other apha or beta glycoprotein subunits to produce a functional glycoprotein hormone with similar, altered or enhanced activity to that of other related glycoprotein hormones (e.g., luteinizing hormone (LH), follicle stimulating hormone (FSH), thyroid stimulating hormone (TSH) and chorionic gonadotropin (hCG). The multimer may be a homopolymer ( i.e., ARP-ARP, BRP- BRP,) or alternatively a heteropolymer with a second polypeptide. The second polypeptide can be from the same species of ARP or BRP, e.g.,. Alternatively, the second polypeptide can be from a different species. Preferably the second polypeptide is. For example, a BRP heteropolymer includes a BRP protein and an alpha glycoprotein subunit or a fragment thereof. Alternatively, the second polypeptide is a cystine knot protein. Examples of an alpha glycoprotein subunit include, GenBank Acession Numbers, AAH10957, and CAC43234. Preferably, the BRP polypeptide forms a multimer with an ARP polypeptide. More preferably, the BRP polypeptide forms a multimer with the polypeptide and biologically active portions thereof, or derivatives, fragments, analogs or homologs thereof of SEQ ID NO: 18. Alternatively, an ARP heteropolymer includes an ARP polypeptide and a beta glycoprotein subunit. Examples of an beta glycoprotein subunit include, GenBank Acession Numbers, P01225 and P18842. Preferably, the ARP polypeptide forms a multimer with an BRP polypeptide.

Also provided are polypeptide fragments suitable for use as immunogens to raise anti-ARP or BRP multimer antibodies. In one embodiment, native ARP or BRP multimer can be isolated from cells or tissue sources by an appropriate purification scheme using standard protein purification techniques. In another embodiment, ARP or BRP multimer is produced by recombinant DNA techniques. Alternative to recombinant expression, a ARP or BRP multimers can be synthesized chemically using standard peptide synthesis techniques. The ARP or BRP multimer may be glycosylated at one or more sites. Alternatively, the ARP or BRP multimer is not glycosylated.

Determining Homology Between Two or more Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 3, 17, 19, or 21.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides ARP/BRP chimeric or fusion proteins. As used herein, a ARP/BRP “chimeric protein” or “fusion protein” comprises a ARP/BRP polypeptide operatively linked to a non-ARP/BRP polypeptide. Alternatively, the ARP/BRP fusion protein is a multimer, e.g., homodimer or heterodimer. A “ARP/BRP polypeptide” refers to a polypeptide having an amino acid sequence corresponding to ARP/BRP, whereas a “non-ARP/BRP polypeptide” refers to a polypeptide having an amino acid sequence corresponding to a protein that is not substantially homologous to the ARP/BRP protein, e.g., a protein that is different from the ARP/BRP protein and that is derived from the same or a different organism. Within a ARP/BRP fusion protein the ARP/BRP polypeptide can correspond to all or a portion of a ARP/BRP protein. In one embodiment, a ARP/BRP fusion protein comprises at least one biologically active portion of a ARP/BRP protein. In another embodiment, a ARP/BRP fusion protein comprises at least two biologically active portions of a ARP/BRP protein. In yet another embodiment, a ARP/BRP fusion protein comprises at least three biologically active portions of a ARP/BRP protein. Within the fusion protein, the term “operatively linked” is intended to indicate that the ARP/BRP polypeptide and the non-ARP/BRP polypeptide are fused in-frame to each other. The non-ARP/BRP polypeptide can be fused to the N-terminus or C-terminus of the ARP/BRP polypeptide.

For example, in one embodiment a ARP/BRP fusion protein comprises a ARP/BRP cystine knot domain or glycoprotein hoermone beta subunit domain operably linked to the extracellular domain of a second protein. Such fusion proteins can be further utilized in screening assays for compounds which modulate ARP/BRP activity (such assays are described in detail below).

In one embodiment, the fusion protein is a GST-ARP/BRP fusion protein in which the ARP/BRP sequences are fused to the C-terminus of the GST (i.e., glutathione S-transferase) sequences. Such fusion proteins can facilitate the purification of recombinant ARP/BRP.

In another embodiment, the fusion protein is a ARP/BRP protein containing a heterologous signal sequence at its N-terminus. For example, the native BRP signal sequence MKLAFLLLGPMALLLLAGYGCLG (SEQ ID NO: 10, i e., about amino acids 1 to 23 of SEQ ID NO: 2) can be removed and replaced with a signal sequence from another protein. Alternatively, the native ARP signal sequence MPMASPQTLVLYLLVLAVTEAWG (SEQ ID NO: 28, i.e., about amino acids 1 to 25 of SEQ ID NO: 18) can be removed and replaced with a signal sequence from another protein. In certain host cells (e.g., mammalian host cells), expression and/or secretion of ARP/BRP can be increased through use of a heterologous signal sequence. In a specific embodiment, the signal sequence of the ARP/BRP protein is removed and replaced with the signal sequence of the hCG beta subunit (MEMFQGLLLLLLLSMGGTWA; SEQ ID NO: 11) to promote the secretion of ARP/BRP.

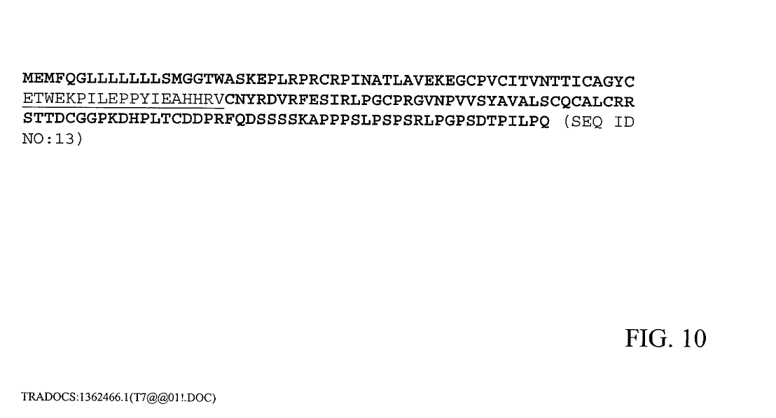

In yet another embodiment, the fusion protein comprises a known glycoprotein hormone with the ARP/BRP loop 2 domain exchanged with the the native loop 2 domain of the hormone. For example, amino acids ETWEKPILEPPYIEAHHRV (SEQ ID NO: 12), comprising the loop 2 domain of a BRP protein can be placed into a CG beta subunit to produce a hCG analog with altered activity. The resulting fusion protein is shown in FIG. 10. (SEQ ID NO: 13)

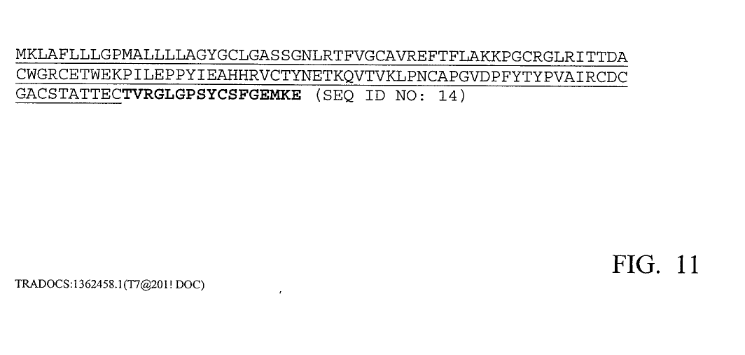

In a further embodiment, the fusion protein is a ARP/BRP protein containing a heterologous seat belt domain from a known glycoprotein hormone beta subunit, e.g., FSH, TSH, LH or hCG. This may for example stabilize the interaction with the alpha subunit or influence receptor binding. For example, the sequence of the BRP protein up to the last cysteine is fused to residues 95-111 from FSH beta (see FIG. 11; SEQ ID NO: 14)). In various embodiments, the ARP/BRP protein is further modified by replacing any amino acid within amino acids 1-75 of the ARP/BRP protein with a cysteine. In one embodiment, the cysteine replaces the glycine at position 51 of BRP. In an alternative embodiment, the cysteine replaces the leucine at position 52 of BRP.

In one embodiment, the fusion protein is a ARP/BRP-immunoglobulin fusion protein in which the ARP/BRP sequences comprising primarily the cystine knot domains are fused to sequences derived from a member of the immunoglobulin protein family. The ARP/BRP-immunoglobulin fusion proteins of the invention can be incorporated into pharmaceutical compositions and administered to a subject to inhibit an interaction between a ARP/BRP ligand and a ARP/BRP protein on the surface of a cell, to thereby suppress ARP/BRP-mediated signal transduction in vivo. The ARP/BRP-immunoglobulin fusion proteins can be used to affect the bioavailability of a ARP/BRP cognate ligand. Inhibition of the ARP/BRP ligand/ARP/BRP interaction may be useful therapeutically for both the treatment of proliferative and differentiative disorders, as well as modulating (e.g. promoting or inhibiting) cell survival. Moreover, the ARP/BRP-immunoglobulin fusion proteins of the invention can be used as immunogens to produce anti-ARP/BRP antibodies in a subject, to purify ARP/BRP ligands, and in screening assays to identify molecules that inhibit the interaction of ARP/BRP with a ARP/BRP ligand.

A ARP/BRP chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A ARP/BRP-encoding nucleic acid can be cloned into such an expression vector such that the fusion moiety is linked in-frame to the ARP/BRP protein.

ARP/BRP Agoni-sts and Antagonists

The present invention also pertains to variants of the ARP/BRP proteins that function as either ARP/BRP agonists (mimetics) or as ARP/BRP antagonists. Variants of the ARP/BRP protein can be generated by mutagenesis, e.g., discrete point mutation or truncation of the ARP/BRP protein. An agonist of the ARP/BRP protein can retain substantially the same, or a subset of, the biological activities of the naturally occurring form of the ARP/BRP protein. An antagonist of the ARP/BRP protein can inhibit one or more of the activities of the naturally occurring form of the ARP/BRP protein by, for example, competitively binding to a downstream or upstream member of a cellular signaling cascade which includes the ARP/BRP protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the ARP/BRP proteins.

Variants of the ARP/BRP protein that function as either ARP/BRP agonists (mimetics) or as ARP/BRP antagonists can be identified by screening combinatorial libraries of mutants, e.g., truncation mutants, of the ARP/BRP protein for ARP/BRP protein agonist or antagonist activity. In one embodiment, a variegated library of ARP/BRP variants is generated by combinatorial mutagenesis at the nucleic acid level and is encoded by a variegated gene library. A variegated library of ARP/BRP variants can be produced by, for example, enzymatically ligating a mixture of synthetic oligonucleotides into gene sequences such that a degenerate set of potential ARP/BRP sequences is expressible as individual polypeptides, or alternatively, as a set of larger fusion proteins (e.g., for phage display) containing the set of ARP/BRP sequences therein. There are a variety of methods which can be used to produce libraries of potential ARP/BRP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ARP/BRP sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the ARP/BRP protein coding sequence can be used to generate a variegated population of ARP/BRP fragments for screening and subsequent selection of variants of a ARP/BRP protein. In one embodiment, a library of coding sequence fragments can be generated by treating a double stranded PCR fragment of a ARP/BRP coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the ARP/BRP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ARP/BRP proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify ARP/BRP variants (Arkin and Yourvan (1992) PNAS 89:7811-7815; Delgrave et al. (1993) Protein Engineering 6:327-331).

Anti-ARP/BRP Antibodies

The invention encompasses antibodies and antibody fragments, such as F_{ab} or (F_{ab})_{2,} that bind immunospecifically to any of the polypeptides, e.g., ARP/BRP protein or ARP/BRP multimers, of the invention.

An isolated ARP/BRP protein, or a portion or fragment thereof, can be used as an immunogen to generate antibodies that bind ARP/BRP using standard techniques for polyclonal and monoclonal antibody preparation. The full-length ARP/BRP protein can be used or, alternatively, the invention provides antigenic peptide fragments of ARP/BRP for use as immunogens. The antigenic peptide of ARP/BRP comprises at least 4 amino acid residues of the amino acid sequence shown in SEQ ID NO: 2, 4, 18, 20, and 22 and encompasses an epitope of ARP/BRP such that an antibody raised against the peptide forms a specific immune complex with ARP/BRP. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

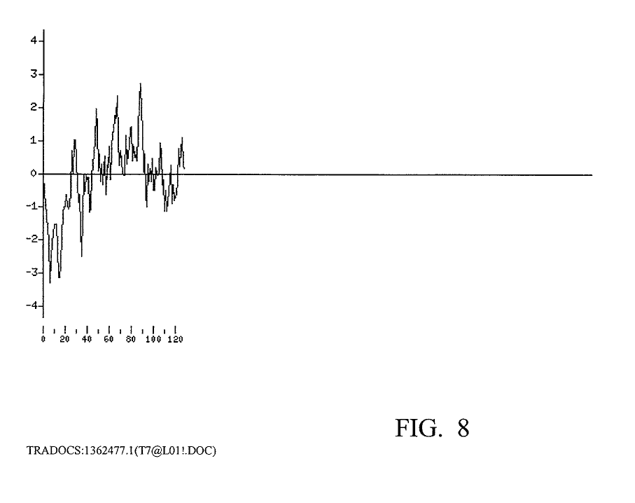

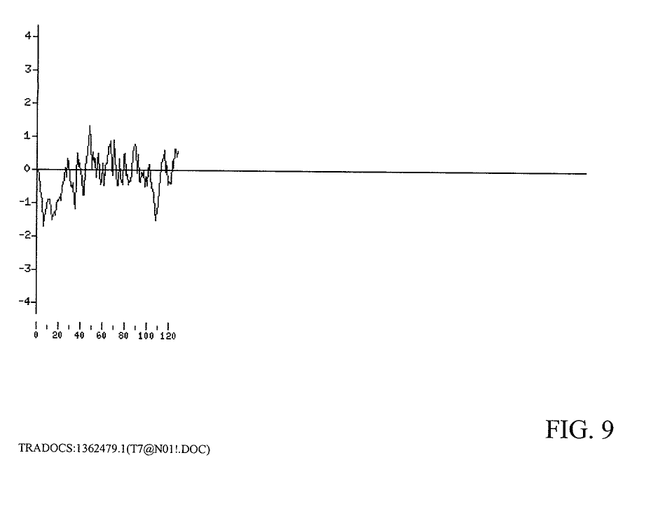

In certain embodiments of the invention, at least one epitope encompassed by the antigenic peptide is a region of ARP/BRP that is located on the surface of the protein, e.g., a hydrophilic region. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J Mol. Biol. 157: 105-142, each incorporated herein by reference in their entirety. Both a Kyte-Doolittle and a Hopp-Woods hydrophobicity analysis of the ARP/BRP protein sequence, as shown in FIGS. 8 and 9 indicate that regions in loop 2 (loop prediction is base on a homology from hCG beta subunit crystal structure) are particularly hydrophilic and, therefore, are likely to encode surface residues useful for targeting antibody production.

In a specific embodiment the antigenic peptide comprises the amino acid sequence CETWEKPILEPPYIEAHHRVC. (SEQ ID NO: 15) In yet another specific embodiment the antigenic peptide comprises the amino acid sequence ETWEKPILEPPYIEAHHRV. (SEQ ID NO: 16)

As disclosed herein, ARP/BRP protein sequence of SEQ ID NO: 2, 4, 18, 20, and 22, or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as ARP/BRP. Such antibodies include, but are not limited to, polyclonal, monoclonal, chimeric, single chain, F_{ab} and F(ab′)₂ fragments, and an F_{ab} expression library. In a specific embodiment, antibodies to ARP/BRP proteins are disclosed. Various procedures known within the art may be used for the production of polyclonal or monoclonal antibodies to a ARP/BRP protein sequence of SEQ ID NO: 2, 4, 18, 20, and 22, or derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed ARP/BRP protein or a chemically synthesized ARP/BRP polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants such as Bacille Calmette-Guerin and Corynebacterium parvum, or similar immunostimulatory agents. If desired, the antibody molecules directed against ARP/BRP can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of ARP/BRP. A monoclonal antibody composition thus typically displays a single binding affinity for a particular ARP/BRP protein with which it immunoreacts. For preparation of monoclonal antibodies directed towards a particular ARP/BRP protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the B-cell hybridoma technique (see Kozbor, et al., 1983 Immunol Today 4: 72) and the EBV hybridoma technique to produce monoclonal antibodies (see Cole, et al., 1985 In: MONOCLONAL ANTIBODIES AND CANCER THERAPY, Alan R. Liss, Inc., pp. 77-96). monoclonal antibodies may be utilized in the practice of the present invention and may be produced by using hybridomas (see Cote, et al., 1983. Proc Natl Acad Sci USA 80: 2026-2030) or by transforming B-cells with Epstein Barr Virus in vitro (see Cole, et al., 1985 In: MONOCLONAL ANTIBODIES AND CANCER THERAPY, Alan R. Liss, Inc., pp. 77-96). Each of the above citations are incorporated herein by reference in their entirety.

According to the invention, techniques can be adapted for the production of single-chain antibodies specific to a ARP/BRP protein (see e.g., U.S. Pat. No. 4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a ARP/BRP protein or derivatives, fragments, analogs or homologs thereof. Non- antibodies can be “humanized” by techniques well known in the art. See e.g., U.S. Pat. No. 5,225,539. Antibody fragments that contain the idiotypes to a ARP/BRP protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-ARP/BRP antibodies, such as chimeric and humanized monoclonal antibodies, comprising both and non-portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison(1985) Science 229:1202-1207; Oi et al. (1986) BioTechniques 4:214; Jones et al. (1986) Nature 321:552-525; Verhoeyan et al. (1988) Science 239:1534; and Beidler et al. (1988) J Immunol 141:4053-4060. Each of the above citations are incorporated herein by reference in their entirety.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a ARP/BRP protein is facilitated by generation of hybridomas that bind to the fragment of a ARP/BRP protein possessing such a domain. Antibodies that are specific for a cystine knot domain within a ARP/BRP protein, or derivatives, fragments, analogs or homologs thereof, are also provided herein.

Anti-ARP/BRP antibodies may be used in methods known within the art relating to the localization and/or quantitation of a ARP/BRP protein (e.g., for use in measuring levels of the ARP/BRP protein within appropriate physiological samples, for use in diagnostic methods, for use in imaging the protein, and the like). In a given embodiment, antibodies for ARP/BRP proteins, or derivatives, fragments, analogs or homologs thereof, that contain the antibody derived binding domain, are utilized as pharmacologically-active compounds [hereinafter “Therapeutics”].

An anti-ARP/BRP antibody (e.g., monoclonal antibody) can be used to isolate ARP/BRP by standard techniques, such as affinity chromatography or immunoprecipitation. An anti-ARP/BRP antibody can facilitate the purification of natural ARP/BRP from cells and of recombinantly produced ARP/BRP expressed in host cells. Moreover, an anti-ARP/BRP antibody can be used to detect ARP/BRP protein (e.g., in a cellular lysate or cell supernatant) in order to evaluate the abundance and pattern of expression of the ARP/BRP protein. Anti-ARP/BRP antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

ARP/BRP Recombinant Expression Vectors and Host Cells

Another aspect of the invention pertains to vectors, preferably expression vectors, containing a nucleic acid encoding ARP/BRP protein ARP/BRP multimers, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA SEQments can be ligated. Another type of vector is a viral vector, wherein additional DNA SEQments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., ARP/BRP proteins, mutant forms of ARP/BRP, fusion proteins, etc.).

The recombinant expression vectors of the invention can be designed for expression of ARP/BRP in prokaryotic or eukaryotic cells. For example, ARP/BRP can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the ARP/BRP expression vector is a yeast expression vector. Examples of vectors for expression in yeast S. cerivisae include pYepSec1 (Baldari, et al., (1987) EMBO J 6:229-234), pMFa (Kuijan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, ARP/BRP can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of 2, directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to ARP/BRP mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, ARP/BRP protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding ARP/BRP or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) ARP/BRP protein. Accordingly, the invention further provides methods for producing ARP/BRP protein using the host cells of the invention. In one embodiment, the method comprises culturing the host cell of invention (into which a recombinant expression vector encoding ARP/BRP has been introduced) in a suitable medium such that ARP/BRP protein is produced. In another embodiment, the method further comprises isolating ARP/BRP from the medium or the host cell.

Transgenic Animals

The host cells of the invention can also be used to produce nonhuman transgenic animals. For example, in one embodiment, a host cell of the invention is a fertilized oocyte or an embryonic stem cell into which ARP/BRP-coding sequences have been introduced. Such host cells can then be used to create non-transgenic animals in which exogenous ARP/BRP sequences have been introduced into their genome or homologous recombinant animals in which endogenous ARP/BRP sequences have been altered. Such animals are useful for studying the function and/or activity of ARP/BRP and for identifying and/or evaluating modulators of ARP/BRP activity. As used herein, a “transgenic animal” is a non-animal, preferably a mammal, more preferably a rodent such as a rat or mouse, in which one or more of the cells of the animal includes a transgene. Other examples of transgenic animals include non-primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a a non-animal, preferably a mammal, more preferably a mouse, in which an endogenous ARP/BRP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing ARP/BRP-encoding nucleic acid into the male pronuclei of a fertilized oocyte, e.g., by microinjection, retroviral infection, and allowing the oocyte to develop in a pseudopregnant female foster animal. The ARP/BRP cDNA sequence of SEQ ID NO: 1, 3, 17, 19, and 21, can be introduced as a transgene into the genome of a non-animal. Alternatively, a nonhuman homologue of the ARP/BRP gene, such as a mouse ARP/BRP gene, can be isolated based on hybridization to the ARP/BRP cDNA (described further above) and used as a transgene. Intronic sequences and polyadenylation signals can also be included in the transgene to increase the efficiency of expression of the transgene. A tissue-specific regulatory sequence(s) can be operably linked to the ARP/BRP transgene to direct expression of ARP/BRP protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the ARP/BRP transgene in its genome and/or expression of ARP/BRP mRNA in tissues or cells of the animals. A transgenic founder animal can then be used to breed additional animals carrying the transgene. Moreover, transgenic animals carrying a transgene encoding ARP/BRP can further be bred to other transgenic animals carrying other transgenes.

To create a homologous recombinant animal, a vector is prepared which contains at least a portion of a ARP/BRP gene into which a deletion, addition or substitution has been introduced to thereby alter, e.g., functionally disrupt, the ARP/BRP gene. The ARP/BRP gene can be a gene (e.g., the cDNA of SEQ ID NO: 1, 3, 17, 19, and 21, but more preferably, is a non-homologue of a ARP/BRP gene. For example, a mouse homologue of ARP/BRP gene of SEQ ID NO: 1, 3, 17, 19, and 21, can be used to construct a homologous recombination vector suitable for altering an endogenous ARP/BRP gene in the mouse genome. In one embodiment, the vector is designed such that, upon homologous recombination, the endogenous ARP/BRP gene is functionally disrupted (i.e., no longer encodes a functional protein; also referred to as a “knock out” vector).

Alternatively, the vector can be designed such that, upon homologous recombination, the endogenous ARP/BRP gene is mutated or otherwise altered but still encodes functional protein (e.g., the upstream regulatory region can be altered to thereby alter the expression of the endogenous ARP/BRP protein). In the homologous recombination vector, the altered portion of the ARP/BRP gene is flanked at its 5′ and 3′ ends by additional nucleic acid of the ARP/BRP gene to allow for homologous recombination to occur between the exogenous ARP/BRP gene carried by the vector and an endogenous ARP/BRP gene in an embryonic stem cell. The additional flanking ARP/BRP nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced ARP/BRP gene has homologously recombined with the endogenous ARP/BRP gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a in recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The ARP/BRP nucleic acid molecules, ARP/BRP proteins, ARP/BRP multimers and anti-ARP/BRP antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ARP/BRP protein or anti-ARP/BRP antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Uses and Methods of the Invention

Soluble proteins containing cystine knot domains such as the glycoprotein hormones and other growth factors are know to bind (i) G-protein coupled receptors, (ii) other cystine knot proteins, (iii) glycoprotein hormone superfamily members, and (iv) tyrosine kinase growth factor receptors. These superfamily members are multifunctional proteins that modulate a number of functions. The nucleic acid molecules, proteins, protein homologues, multimers and antibodies described herein that include cystine knot domains, therefore, can be used in one or more of the following methods: (a) screening assays; (b) detection assays (e.g., tissue typing), (c) predictive medicine (e.g., diagnostic assays, prognostic assays, monitoring clinical trials, and pharmacogenomics); and (d) methods of treatment (e.g., therapeutic and prophylactic). A ARP/BRP protein interacts with other cellular proteins and can thus be used for (i) modulation of ARP/BRP-related protein activity; (ii) regulation of cellular proliferation; (iii) regulation of cellular differentiation; and (iv) regulation of reproductive functions.

The isolated nucleic acid molecules of the invention can be used to express ARP/BRP protein (e.g., via a recombinant expression vector in a host cell in gene therapy applications), to detect ARP/BRP mRNA (e.g., in a biological sample) or a genetic lesion in a ARP/BRP gene, and to modulate ARP/BRP activity, as described further below. In addition, the ARP/BRP proteins can be used to screen drugs or compounds that modulate the ARP/BRP polypeptide, multimer or nucleic acid activity or expression as well as to treat disorders characterized by insufficient or excessive production of ARP/BRP protein or multimers or production of ARP//BRP protein or multimer forms that have decreased or aberrant activity compared to ARP/BRP wild type protein or multimer (e.g. proliferative disorders such as cancer, ovulatory disorders, infertility, hypogonadism or metabolic disorder effecting pituitary function or pituitary target organs such as for example, adrenal gland, thyroid, gonad or liver). In addition, the anti-ARP/BRP antibodies of the invention can be used to detect and isolate ARP/BRP proteins and modulate ARP/BRP activity.

This invention further pertains to novel agents identified by the above described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) that bind to ARP/BRP proteins or ARP/BRP multimers or have a stimulatory or inhibitory effect on, for example, ARP/BRP expression or ARP/BRP activity.

In one embodiment, the invention provides assays for screening candidate or test compounds which bind to or modulate the activity of the membrane-bound form of a ARP/BRP protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1 992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of ARP/BRP protein or ARP/BRP multimer, or a biologically active portion thereof, on the cell surface is contacted with a test compound and the ability of the test compound to bind to a ARP/BRP protein or multimer is determined. The cell, for example, can of mammalian origin or a yeast cell. Determining the ability of the test compound to bind to the ARP/BRP protein or multimer can be accomplished, for example, by coupling the test compound with a radioisotope or enzymatic label such that binding of the test compound to the ARP/BRP protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of ARP/BRP protein, or a biologically active portion thereof, on the cell surface with a known compound which binds ARP/BRP to form an assay mixture, contacting the assay mixture with a test compound, and determining the ability of the test compound to interact with a ARP/BRP protein, wherein determining the ability of the test compound to interact with a ARP/BRP protein comprises determining the ability of the test compound to preferentially bind to ARP/BRP or a biologically active portion thereof as compared to the known compound.

In another embodiment, an assay is a cell-based assay comprising contacting a cell expressing a membrane-bound form of ARP/BRP protein, or multimer or a biologically active portion thereof, on the cell surface with a test compound and determining the ability of the test compound to modulate (e.g., stimulate or inhibit) the activity of the ARP/BRP protein or multimer or biologically active portion thereof. Determining the ability of the test compound to modulate the activity of ARP/BRP or a biologically active portion thereof can be accomplished, for example, by determining the ability of the ARP/BRP protein to bind to or interact with a ARP/BRP target molecule. As used herein, a “target molecule” is a molecule with which a ARP/BRP protein binds or interacts in nature, for example, a molecule on the surface of a cell which expresses a ARP/BRP interacting protein, a molecule on the surface of a second cell, a molecule in the extracellular milieu, a molecule associated with the internal surface of a cell membrane or a cytoplasmic molecule. A ARP/BRP target molecule can be a non-ARP/BRP molecule or a ARP/BRP protein or polypeptide of the present invention. In one embodiment, a ARP/BRP target molecule is a component of a signal transduction pathway that facilitates transduction of an extracellular signal (e.g. a signal generated by binding of a compound to a membrane-bound ARP/BRP molecule) through the cell membrane and into the cell. The target, for example, can be a second intercellular protein that has catalytic activity or a protein that facilitates the association of downstream signaling molecules with ARP/BRP.

Determining the ability of the ARP/BRP protein to bind to or interact with a ARP/BRP target molecule can be accomplished by one of the methods described above for determining direct binding. In one embodiment, determining the ability of the ARP/BRP protein to bind to or interact with a ARP/BRP target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a ARP/BRP-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a ARP/BRP protein or biologically active portion thereof with a test compound and determining the ability of the test compound to bind to the ARP/BRP protein or biologically active portion thereof. Binding of the test compound to the ARP/BRP protein can be determined either directly or indirectly as described above. In one embodiment, the assay comprises contacting the ARP/BRP protein or biologically active portion thereof with a known compound which binds ARP/BRP to form an assay mixture, contacting the assay mixture with a test compound, and determining the ability of the test compound to interact with a ARP/BRP protein, wherein determining the ability of the test compound to interact with a ARP/BRP protein comprises determining the ability of the test compound to preferentially bind to ARP/BRP or biologically active portion thereof as compared to the known compound.

In another embodiment, an assay is a cell-free assay comprising contacting ARP/BRP protein or biologically active portion thereof with a test compound and determining the ability of the test compound to modulate (e.g. stimulate or inhibit) the activity of the ARP/BRP protein or biologically active portion thereof. Determining the ability of the test compound to modulate the activity of ARP/BRP can be accomplished, for example, by determining the ability of the ARP/BRP protein to bind to a ARP/BRP target molecule by one of the methods described above for determining direct binding. In an alternative embodiment, determining the ability of the test compound to modulate the activity of ARP/BRP can be accomplished by determining the ability of the ARP/BRP protein further modulate a ARP/BRP target molecule. For example, the catalytic/enzymatic activity of the target molecule on an appropriate substrate can be determined as previously described.

In yet another embodiment, the cell-free assay comprises contacting the ARP/BRP protein or biologically active portion thereof with a known compound which binds ARP/BRP to form an assay mixture, contacting the assay mixture with a test compound, and determining the ability of the test compound to interact with a ARP/BRP protein, wherein determining the ability of the test compound to interact with a ARP/BRP protein comprises determining the ability of the ARP/BRP protein to preferentially bind to or modulate the activity of a ARP/BRP target molecule.

The cell-free assays of the present invention are amenable to use of both the soluble form or the membrane-bound form of ARP/BRP. In the case of cell-free assays comprising the membrane-bound form of ARP/BRP, it may be desirable to utilize a solubilizing agent such that the membrane-bound form of ARP/BRP is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either ARP/BRP or its target molecule to facilitate separation of complexed from uncomplexed forms of one or both of the proteins, as well as to accommodate automation of the assay. Binding of a test compound to ARP/BRP, or interaction of ARP/BRP with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-ARP/BRP fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or ARP/BRP protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of ARP/BRP binding or activity determined using standard techniques.

Other techniques for immobilizing proteins on matrices can also be used in the screening assays of the invention. For example, either ARP/BRP or its target molecule can be immobilized utilizing conjugation of biotin and streptavidin. Biotinylated ARP/BRP or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with ARP/BRP or target molecules, but which do not interfere with binding of the ARP/BRP protein to its target molecule, can be derivatized to the wells of the plate, and unbound target or ARP/BRP trapped in the wells by antibody conjugation. Methods for detecting such complexes, in addition to those described above for the GST-immobilized complexes, include immunodetection of complexes using antibodies reactive with the ARP/BRP or target molecule, as well as enzyme-linked assays that rely on detecting an enzymatic activity associated with the ARP/BRP or target molecule.

In another embodiment, modulators of ARP/BRP expression are identified in a method wherein a cell is contacted with a candidate compound and the expression of ARP/BRP mRNA or protein in the cell is determined. The level of expression of ARP/BRP mRNA or protein in the presence of the candidate compound is compared to the level of expression of ARP/BRP mRNA or protein in the absence of the candidate compound. The candidate compound can then be identified as a modulator of ARP/BRP expression based on this comparison. For example, when expression of ARP/BRP mRNA or protein is greater (statistically significantly greater) in the presence of the candidate compound than in its absence, the candidate compound is identified as a stimulator of ARP/BRP mRNA or protein expression. Alternatively, when expression of ARP/BRP mRNA or protein is less (statistically significantly less) in the presence of the candidate compound than in its absence, the candidate compound is identified as an inhibitor of ARP/BRP mRNA or protein expression. The level of ARP/BRP mRNA or protein expression in the cells can be determined by methods described herein for detecting ARP/BRP mRNA or protein.

In yet another aspect of the invention, the ARP/BRP proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with ARP/BRP (“ARP/BRP-binding proteins” or “ARP/BRP-bp”) and modulate ARP/BRP activity. Such ARP/BRP-binding proteins are also likely to be involved in the propagation of signals by the ARP/BRP proteins as, for example, upstream or downstream elements of the ARP/BRP pathway.

The two-hybrid system is based on the modular nature of most transcription factors, which consist of separable DNA-binding and activation domains. Briefly, the assay utilizes two different DNA constructs. In one construct, the gene that codes for ARP/BRP is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a ARP/BRP-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with ARP/BRP.

This invention further pertains to novel agents identified by the above-described screening assays and uses thereof for treatments as described herein.

Tissue Typing

The ARP/BRP sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the ARP/BRP sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The ARP/BRP sequences of the invention uniquely represent portions of the genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, 3, 17, 19, and 21 can comfortably provide positive individual identification with a panel of perhaps 10 to 1,000 primers that each yield a noncoding amplified sequence of 100 bases. If predicted coding sequences, such as those in SEQ ID NO: 2, 4, 18, 20, and 22 are used, a more appropriate number of primers for positive individual identification would be 500-2,000.

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining ARP/BRP protein, ARP/BRP multimer and/or nucleic acid expression as well as ARP/BRP or ARP/BRP multimer activity, in the context of a biological sample (e.g., blood, serum, cells, tissue) to thereby determine whether an individual is afflicted with a disease or disorder, or is at risk of developing a disorder, associated with aberrant ARP/BRP expression or activity, e.g. reproductive disorders, infertility, ovulatory disorders. The invention also provides for prognostic (or predictive) assays for determining whether an individual is at risk of developing a disorder associated with ARP/BRP protein, multimer nucleic acid expression or activity. For example, mutations in a ARP/BRP gene can be assayed in a biological sample. Such assays can be used for prognostic or predictive purpose to thereby prophylactically treat an individual prior to the onset of a disorder characterized by or associated with ARP/BRP protein, nucleic acid expression or activity.

Another aspect of the invention provides methods for determining ARP/BRP protein, multimer nucleic acid expression or ARP/BRP activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of ARP/BRP in clinical trials.

These and other agents are described in further detail in the following sections.

Diagnostic Assays

An exemplary method for detecting the presence or absence of ARP/BRP in a biological sample involves obtaining a biological sample from a test subject and contacting the biological sample with a compound or an agent capable of detecting ARP/BRP protein, ARP/BRP multimer or nucleic acid (e.g., mRNA, genomic DNA) that encodes ARP/BRP protein such that the presence of ARP/BRP is detected in the biological sample. An agent for detecting ARP/BRP mRNA or genomic DNA is a labeled nucleic acid probe capable of hybridizing to ARP/ BRP mRNA or genomic DNA. The nucleic acid probe can be, for example, a full-length ARP/BRP nucleic acid, such as the nucleic acid of SEQ ID NO: 1, 3, 17, 19, and 21, or a portion thereof, such as an oligonucleotide of at least 15, 30, 50, 100, 250 or 500 nucleotides in length and sufficient to specifically hybridize under stringent conditions to ARP/BRP mRNA or genomic DNA. Other suitable probes for use in the diagnostic assays of the invention are described herein.

An agent for detecting ARP/BRP protein or ARP/BRP multimer is an antibody capable of binding to ARP/BRP protein or ARP/BRP multimer, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect ARP/BRP mRNA, protein, or genomic DNA in a biological sample in vitro as well as in vivo. For example, in vitro techniques for detection of ARP/BRP mRNA include Northern hybridizations and in situ hybridizations. In vitro techniques for detection of ARP/BRP protein include enzyme linked immunosorbent assays (ELISAs), Western blots, immunoprecipitations and immunofluorescence. In vitro techniques for detection of ARP/BRP genomic DNA include Southern hybridizations. Furthermore, in vivo techniques for detection of ARP/BRP protein include introducing into a subject a labeled anti-ARP/BRP antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting ARP/BRP protein, multimers, mRNA, or genomic DNA, such that the presence of ARP/BRP protein, multimers, mRNA or genomic DNA is detected in the biological sample, and comparing the presence of ARP/BRP protein, mRNA or genomic DNA in the control sample with the presence of ARP/BRP protein, multimers, mRNA or genomic DNA in the test sample.

The invention also encompasses kits for detecting the presence of ARP/BRP in a biological sample. For example, the kit can comprise: a labeled compound or agent capable of detecting ARP/BRP protein, multimer or mRNA in a biological sample; means for determining the amount of ARP/BRP in the sample; and means for comparing the amount of ARP/BRP in the sample with a standard. The compound or agent can be packaged in a suitable container. The kit can further comprise instructions for using the kit to detect ARP/BRP protein or nucleic acid.

Prognostic Assays

The diagnostic methods described herein can furthermore be utilized to identify subjects having or at risk of developing a disease or disorder associated with aberrant ARP/BRP expression or activity. For example, the assays described herein, such as the preceding diagnostic assays or the following assays, can be utilized to identify a subject having or at risk of developing a disorder associated with ARP/BRP protein, multimer, nucleic acid expression or activity such as cancer, ovulatory disorders, infertility or hypogonadism. Alternatively, the prognostic assays can be utilized to identify a subject having or at risk for developing a disease or disorder. Thus, the present invention provides a method for identifying a disease or disorder associated with aberrant ARP/BRP expression or activity in which a test sample is obtained from a subject and ARP/BRP protein, multimer or nucleic acid (e.g., mRNA, genomic DNA) is detected, wherein the presence of ARP/BRP protein, multimer or nucleic acid is diagnostic for a subject having or at risk of developing a disease or disorder associated with aberrant ARP/BRP expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant ARP/BRP expression or activity. For example, such methods can be used to determine whether a subject can be effectively treated with an agent for a disorder, such as cancer, ovulatory disorders, infertility or hypogonadism. Thus, the present invention provides methods for determining whether a subject can be effectively treated with an agent for a disorder associated with aberrant ARP/BRP expression or activity in which a test sample is obtained and ARP/BRP protein or nucleic acid is detected (e.g., wherein the presence of ARP/BRP protein or nucleic acid is diagnostic for a subject that can be administered the agent to treat a disorder associated with aberrant ARP/BRP expression or activity.)

The methods of the invention can also be used to detect genetic lesions in a ARP/BRP gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a ARP/BRP-protein, or the mis-expression of the ARP/BRP gene. For example, such genetic lesions can be detected by ascertaining the existence of at least one of (1) a deletion of one or more nucleotides from a ARP/BRP gene; (2) an addition of one or more nucleotides to a ARP/BRP gene; (3) a substitution of one or more nucleotides of a ARP/BRP gene, (4) a chromosomal rearrangement of a ARP/BRP gene; (5) an alteration in the level of a messenger RNA transcript of a ARP/BRP gene, (6) aberrant modification of a ARP/BRP gene, such as of the methylation pattern of the genomic DNA, (7) the presence of a non-wild type splicing pattern of a messenger RNA transcript of a ARP/BRP gene, (8) a non-wild type level of a ARP/BRP-protein, (9) allelic loss of a ARP/BRP gene, and (10) inappropriate post-translational modification of a ARP/BRP-protein. As described herein, there are a large number of assay techniques known in the art which can be used for detecting lesions in a ARP/BRP gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the ARP/BRP-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a ARP/BRP gene under conditions such that hybridization and amplification of the ARP/BRP gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a ARP/BRP gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in ARP/BRP can be identified by hybridizing a sample and control nucleic acids, e.g., DNA or RNA, to high density arrays containing hundreds or thousands of oligonucleotides probes (Cronin et al. (1996) Mutation 7: 244-255; Kozal et al. (1996) Nature Medicine 2: 753-759). For example, genetic mutations in ARP/BRP can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ARP/BRP gene and detect mutations by comparing the sequence of the sample ARP/BRP with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the ARP/BRP gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type ARP/BRP sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in ARP/BRP cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a ARP/BRP sequence, e.g., a wild-type ARP/BRP sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in ARP/BRP genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control ARP/BRP nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6:1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a ARP/BRP gene.

Furthermore, any cell type or tissue, preferably peripheral blood leukocytes, in which ARP/BRP is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on ARP/BRP or ARP/BRP multimer activity (e.g., ARP/BRP gene expression), as identified by a screening assay described herein can be administered to individuals to treat (prophylactically or therapeutically) disorders (e.g., cancer, ovulatory disorders, infertility or hypogonadism) associated with aberrant ARP/BRP activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ARP/BRP protein, expression of ARP/BRP nucleic acid, or mutation content of ARP/BRP genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of ARP/BRP protein, ARP/BRP multimer, expression of ARP/BRP nucleic acid, or mutation content of ARP/BRP genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a ARP/BRP modulator, such as a modulator identified by one of the exemplary screening assays described herein.

Monitoring of Effects During Clinical Trials

Monitoring the influence of agents (e.g., drugs, compounds) on the expression or activity of ARP/BRP or ARP/BRP multimer (e.g., the ability to modulate aberrant cell proliferation and/or differentiation) can be applied not only in basic drug screening, but also in clinical trials. For example, the effectiveness of an agent determined by a screening assay as described herein to increase ARP/BRP gene expression, protein levels, or upregulate ARP/BRP activity, can be monitored in clinical trails of subjects exhibiting decreased ARP/BRP gene expression, protein levels, or downregulated ARP/BRP activity. Alternatively, the effectiveness of an agent determined by a screening assay to decrease ARP/BRP gene expression, protein levels, or downregulate ARP/BRP activity, can be monitored in clinical trails of subjects exhibiting increased ARP/BRP gene expression, protein levels, or upregulated ARP/BRP activity. In such clinical trials, the expression or activity of ARP/BRP and, preferably, other genes that have been implicated in, for example, cancer, ovulatory disorders, infertility or hypogonadism can be used as a “read out” or markers of the immune responsiveness of a particular cell.

For example, and not by way of limitation, genes, including ARP/BRP, that are modulated in cells by treatment with an agent (e.g., compound, drug or small molecule) that modulates ARP/BRP activity (e.g., identified in a screening assay as described herein) can be identified. Thus, to study the effect of agents on cellular proliferation disorders, for example, in a clinical trial, cells can be isolated and RNA prepared and analyzed for the levels of expression of ARP/BRP and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of ARP/BRP or other genes. In this way, the gene expression pattern can serve as a marker, indicative of the physiological response of the cells to the agent. Accordingly, this response state may be determined before, and at various points during, treatment of the individual with the agent.

In one embodiment, the present invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a ARP/BRP protein, mRNA, or genomic DNA in the preadministration sample; (iii) obtaining one or more post-administration samples from the subject; (iv) detecting the level of expression or activity of the ARP/BRP protein, mRNA, or genomic DNA in the post-administration samples; (v) comparing the level of expression or activity of the ARP/BRP protein, mRNA, or genomic DNA in the pre-administration sample with the ARP/BRP protein, mRNA, or genomic DNA in the post administration sample or samples; and (vi) altering the administration of the agent to the subject accordingly. For example, increased administration of the agent may be desirable to increase the expression or activity of ARP/BRP to higher levels than detected, i e., to increase the effectiveness of the agent. Alternatively, decreased administration of the agent may be desirable to decrease expression or activity of ARP/BRP to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant ARP/BRP expression or activity, e.g. cell proliferative disorders or reproductive disorders. In addition, the BRP and ARP nucleic acid, polypeptides and protein multimers can be used to stimulate spermatogenesis, increase the function of the thyroid glandular cells (i.e., increase thyroid hormone production and iodide trapping), regulate gonadal function, regulate gonadal hormone production and promote or suppress fertility.

Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989, Science 244: 1288-1292); (v) modulators ( i.e., inhibitors, agonists and antagonists, including additional peptide mimetic of the invention or antibodies specific to a peptide of the invention) that alter the interaction between an aforementioned peptide and its binding partner or (vi) an aforementioned protein multimer.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic Methods

In one aspect, the invention provides a method for preventing, in a subject, a disease or condition associated with an aberrant ARP/BRP or expression or activity, by administering to the subject an agent that modulates ARP/BRP expression or at least one ARP/BRP activity. Subjects at risk for a disease that is caused or contributed to by aberrant ARP/BRP expression or activity can be identified by, for example, any or a combination of diagnostic or prognostic assays as described herein. Administration of a prophylactic agent can occur prior to the manifestation of symptoms characteristic of the ARP/BRP aberrancy, such that a disease or disorder is prevented or, alternatively, delayed in its progression. Depending on the type of ARP/BRP aberrancy, for example, a ARP/BRP agonist or ARP/BRP antagonist agent can be used for treating the subject. The appropriate agent can be determined based on screening assays described herein. The prophylactic methods of the present invention are further discussed in the following subsections.

Therapeutic Methods

Another aspect of the invention pertains to methods of modulating ARP/BRP expression or activity for therapeutic purposes. The modulatory method of the invention involves contacting a cell with an agent that modulates one or more of the activities of ARP/BRP protein activity associated with the cell. An agent that modulates ARP/BRP protein activity can be an agent as described herein, such as a nucleic acid or a protein, a naturally-occurring cognate ligand of a ARP/BRP protein, a peptide, a ARP/BRP peptidomimetic, or other small molecule. In one embodiment, the agent stimulates one or more ARP/BRP protein activity. Examples of such stimulatory agents include active ARP/BRP protein and a nucleic acid molecule encoding ARP/BRP that has been introduced into the cell. In another embodiment, the agent inhibits one or more ARP/BRP protein activity. Examples of such inhibitory agents include antisense ARP/BRP nucleic acid molecules and anti-ARP/BRP antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a ARP/BRP protein or nucleic acid molecule. In one embodiment, the method involves administering an agent (e.g., an agent identified by a screening assay described herein), or combination of agents that modulates (e.g., upregulates or downregulates) ARP/BRP expression or activity. In another embodiment, the method involves administering a ARP/BRP protein or nucleic acid molecule as therapy to compensate for reduced or aberrant ARP/BRP expression or activity.

Stimulation of ARP/BRP activity is desirable in situations in which ARP/BRP is abnormally downregulated and/or in which increased ARP/BRP activity is likely to have a beneficial effect. One example of such a situation is where a subject has a disorder characterized by aberrant cell proliferation and/or differentiation (e.g., cancer). Another example of such a situation is where the subject has a reproductive disorder (e.g., ovulatory disorders, or infertility).

Determination of the Biological Effect of the Therapeutic

In various embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in subjects. Similarly, for in vivo testing, any of the animal model system known in the art may be used prior to administration to subjects.

Reproductive Disorders

An aforementioned protein and multimer is involved in reproductive function. Accordingly, Therapeutics of the present invention may be useful in the therapeutic or prophylactic treatment of reproductive diseases or disorders. Reproductive disorders include both female and male reproductive disorders. Examples of female reproductive disorders include, Ovulatory Disorders (i.e., stimulating follicular development and triggering ovulation), premenstrual syndrome, ovarian cysts, endometriosis, uterine leiomyomas, infertility, pelvic inflammatory disease, vaginismus and menopause. Examples of male reproductive disorders include, penile disorder, scrotum disorders, prostate disorders and infertility.

In addition, the BRP and ARP nucleic acid, polypeptides and protein multimers can be used to stimulate spermatogenesis, increase the function of the thyroid glandular cells (i.e., increase thyroid hormone production and iodide trapping), regulate gonadal function, regulate gonadal hormone production and promote or suppress fertility.

Malignancies

An aforementioned protein and multimer is involved in the regulation of cell proliferation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J. B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

Premalignant Conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W. B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDal cell-surface protein, and the like. See e.g., Richards, et al., 1986. MOLECULAR PATHOLOGY, W. B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myelogenous leukemia and t(14;18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a patient to prevent the progression to breast, colon, ovarian, lung, pancreatic, or uterine cancer, or melanoma or sarcoma.

Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Cytokine and Cell Proliferation/Differentiation Activity

A ARP/BRP protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnoili et al., J Immunol 145:1706-1712, 1990; Bertagnolli et al., Cell Immunol 133:327-341, 1991; Bertagnolli, et al., J Immunol 149:3778-3783, 1992; Bowman et al., J Immunol 152:1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith et al, Proc Natl Acad Sci U.S.A. 83:1857-1861, 1986; Measurement of Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405-411, 1981; Takai et al., J Immunol 137:3494-3500, 1986; Takai et al., J Immunol 140:508-512, 1988.

Immune Stimulating or Suppressing Activity

A ARP/BRP protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of autoimmune diseases. Examples include murine experimental autoimmune encephalitis, systemic lupus erythematosis in MRL/lpr/lpr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class a chain protein and β₂ microglobulin protein or an MHC class II a chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Takai et al., J Immunol 140:508-512, 1988; Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Bowman et al., J Virology 61:1992-1998;

Takai et al., J Immunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al., J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virol 67:4062-4069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al., Blood 84:111-117, 1994; Fine et al., Cell Immunol 155: 111-122, 1994; Galy et al., Blood 85:2770-2778, 1995; Toki et al., Proc Nat Acad Sci USA 88:7548-7551, 1991.

Tissue Growth Activity

An ARP/BRP protein or ARP/BRP multimers of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendonitis, cARP/BRPal tunnel syndrome and other tendon or ligament defects. The compositions may also include an appropriate matrix and/or sequestering agent as a career as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, EPIDERMAL WOUND HEALING, pp. 71-112 (Maibach and Rovee, eds.), Year Book Medical Publishers, Inc., Chicago, as modified by Eaglstein and Menz, J. Invest. Dermatol 71:382-84 (1978).

Activin/Inhibin Activity

An ARP/BRP protein or ARP/BRP multimer of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heteromultimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc Natl Acad Sci USA 83:3091-3095, 1986.

Chemotactic/Chemokinetic Activity

A protein or multimer of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95:1370-1376, 1995; Lind et al. APMIS 103:140-146, 1995; Muller et al., Eur J Immunol 25: 1744-1748; Gruberet al. J Immunol 152:5860-5867, 1994; Johnston et al., J Immunol 153: 1762-1768, 1994.

Receptor/Ligand Activity

A protein or multimer of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell—cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868, 1987; Bierer et al., J. Exp. Med. 168:1145-1156, 1988; Rosenstein et al., J. Exp. Med. 169:149-160 1989; Stoltenborg et al., J Immunol Methods 175:59-68, 1994; Stitt et al., Cell 80:661-670, 1995.

Anti-Inflammatory Activity

Proteins or multimers of the present invention may also exhibit anti-inflammatory activity. The anti-inflammatory activity may be achieved by providing a stimulus to cells involved in the inflammatory response, by inhibiting or promoting cell—cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be usefull to treat anaphylaxis and hypersensitivity to an antigenic substance or material.

Tumor Inhibition Activity

In addition to the activities described above for immunological treatment or prevention of tumors, a protein or multimer of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

### EXAMPLES

Brief descriptions of specific terms and procedures frequently used in the examples are t5 provided below.

“PCR” is the polymerase chain reaction.

“cDNA” is complementary DNA synthesized from total or poly(A)+ RNA by reverse transcriptase.

“Digestion” of DNA was done with restriction endonucleases purchased usually from New England Biolabs (Beverly, Mass.). Buffers and reaction conditions were similar to those specified by the manufacturer.

“Gel-purification” of PCR DNA fragments and DNA fragments resulting from restriction endonuclease digestion was done by size-fractionating the reaction mix by electrophoresis in agarose gels with appropriate molecular weight standards. After electrophoresis, the DNA was visualized by staining with ethidium bromide and the fragment of the desired size was excised from the gel and then separated from the agarose using a Wizard® PCR Preps DNA purification system (Promega Corporation, Madison, Wis.).

“Ligation” or “insertion” of a purified DNA fragment(s) into digested and purified vector DNA was done using T4 DNA ligase from New England Biolabs (Beverly, Mass.) with the buffer supplied by the manufacturer. Ligations were also done with Topoisomerase I, using vectors and reagents supplied in kits from Invitrogen (Carlsbad, Calif.).

“Cloning” refers to transformation of suitable E. coli host strains with DNA from ligation reactions, propagating the transformed strains, and purifying plasmid DNA from the bacteria using kits from Qiagen Inc., Santa Clarita, Calif.

“DNA sequencing” was done using the plasmid DNA template, DNA oligonucleotide primers, (such as T7, T3, M13F, M13R, and gene-specific primers), and reagent from the ABI PRISM® BigDye™ Terminator Cycle Sequencing Ready Reaction kit (Applied Biosystems, Foster City, Calif.) in a cycle sequencing reaction. Reaction conditions and cycling conditions were done according to specifications supplied in the kit. An ABI PRISM® 310 Genetic Analyzer (Applied Biosystems, Foster City, Calif.) was used for capillary electrophoresis and raw DNA sequence determination. Sequencher (version 4.0.5, Gene Codes Corporation) and GCG (Wisconsin Package Version 10.1, Genetics Computer Group, Madison, Wis.) software were used for fragment assemblies and DNA sequence alignments.

“TaqMan®” fluorogenic 5′ nuclease assays were done with an ABI PRISM® 7700 Sequence Detection System (Applied Biosystems, Foster City, Calif.). Reactions were done in 1× universal mix (Applied Biosystems, Foster City, Calif.) with 300 or 900 nmole forward and reverse primers, 250 nmole probe, and various amounts of cDNA prepared from total or poly(A)+RNA in a total reaction volume of 25 μl. The universal cycling parameters were used for PCR (50° C., 10 min; 95° C. 10 min, followed by 40 cycles of 95° C. 15 sec, 60° C. 1 min). Primers and probes for the TaqMan® assays were designed using Primer Express Version 1.5® Oligo Design software (Applied Biosystems, Foster City, Calif.).

“Crude Culture Supernatant Concentration” refers to any process whereby the volume of particular sample of conditioned medium is reduced, without significant loss of protein, thereby enriching the sample with respect to protein content. The method used for this work was tangential flow filtration across a polyethersulfone low protein binding membrane of nominal molecular weight cutoff 10,000 Da (Pall Filtron Ultrasette P/N 05010C70). Driving force was provided with a Masterflex L/S peristaltic pump drive and a Model 7518-10 easy-load pump head plumbed with PharMed size 15 thick-walled tubing. The circulation rate was 900-1100 ml/min and backpressure was maintained at ˜1.5 atm through the use a variable outflow restriction. Culture supernatant volumes were reduced 25-50 fold to generate the starting material for immunoaffinity purification.

“SDS-PAGE” refers to a family of techniques by which a dodecyl sulfate detergent-treated protein sample is fractionated, on the basis of mobility in a polymer gel matrix, in response to the driving force of an applied electric field.

“Western Blotting” refers to any technique in which electrophoretically separated proteins that have been transferred to a membrane substratum are subsequently detected by binding to a specific antibody. In this work electrophoretic transfer to polyvinylidene fluoride (PVDF) membrane was used in conjunction with a variety of detection antibodies, as specificed in individual figures.

“Immunoaffinity Chromatography” is the process whereby a sample is fractionated based upon binding affinity of one or more of the sample components for an immobilized antibody. In principle, either competing ligand or reversible denaturation of the immobilized antibody by a sudden pH shift can be used to recover bound sample. For this work captured protein was eluted from immobilized antibody by a pH shift from 7.5 to 3.5. Fractions containing eluted protein were monitored with pH paper and immediately neutralized by the addition of an appropriate volume of 1 M Tris pH 9.2. Immediately following the elution of bound protein the chromatography resin was equilibrated back to pH 7.5 to minimize irreversible denaturation of the immobilized protein.

### Example 1

Detection of Human BRP Transcripts in RNA Extracted from Tissues

Poly(A)+RNA purified from testis, pituitary, liver, thyroid, kidney, pancreas, and K-562 (chronic mylogenous leukemia) was purchased from Clontech (Palo Alto, Calif.). The RNA was used to synthesize complementary DNA (cDNA) using the SUPERSCRIPT™ First-Strand Synthesis System for RT-PCR from Life Technologies, Inc. (cat #11904-018) according to the manufacturer's recommendations, with 0.5-1 μg RNA and 50 ng random hexamers per 20 μl reaction volume. Control reactions, identical except that the reverse transcriptase was omitted, were done to monitor PCR priming from residual genomic DNA in the RNA preparations. Reactions done with or without reverse transcriptase were designated “+RT” or “−RT”, respectively.

The BRP forward and reverse primers used for the TaqMan® assay were 5′-GGGCCTTCGGATCACCAC-3′ (SEQ ID NO: 76) and 5′-TCGATGATGGGCTTCAATATAGG-3′ (SEQ ID NO: 46), respectively. The probe for ARP/BRP was 5′-6FAM-CCTGGGAGAAACCCATTCTGGAACCC-TAMRA-3′(SEQ ID NO: 47). The fluorescent probe spanned the predicted junction of the two coding exons for BRP, thus the assay is intended to specifically detect the spliced mRNA transcript. TaqMan PCR was done was template cDNA prepared from 25 ng or 50 ng poly(A)+RNA in a total reaction volume of 25 μl.

Results of real time quantitative PCR experiments using the BRP TaqMan® assay are shown Table 5.

TABLE 5
Detection of human BRP mRNA in tissues using TaqMan ®
real time quantitative PCR.

| | | Amount of input | | |
|---|---|---|---|---|
| Sample | | RNA per reaction | C_{T}¹ for | C_{T} for |
| # | Tissue/cell line | (ng) | +RT reaction | −RT reaction |
| 1 | K562 cDNA | 50 | 39.48 ± 0.45 | 40 |
| 2 | Pituitary cDNA | 50 | 30.41 ± 0.03 | 38.61 ± 0.84 |
| 3 | Testis cDNA | 50 | 31.34 ± 0.65 | 39.18 ± 1.41 |
| 4 | Pituitary cDNA | 25 | 31.64 ± 0.44 | 38.97 ± 1.79 |
| 5 | Testis cDNA | 25 | 32.11 ± 0.72 | 40 |
| 6 | Kidney cDNA | 25 | 40 | 40 |
| 7 | Liver cDNA | 25 | 38.77 ± 2.14 | 39.20 ± 1.39 |
| 8 | Pancreas | 25 | 39.21 ± 1.37 | 40 |
| | cDNA | | | |
| 9 | Placenta cDNA | 25 | 38.09 ± 1.77 | 40 |

| | | | |
|---|---|---|---|
| 10 | No Template | Not applicable | 40 |
| | Control | | |

¹C_{T}: The PCR cycle at which the normalized reporter fluorescence reaches a defined threshold value.
Lower C_{T} values indicate higher levels of target cDNA.
A C_{T} of >36 was considered negative.

The results show that spliced human BRP mRNA transcripts can be detected reproducibly in pituitary and testis poly(A)+RNA. Although BRP transcripts were not detected in the other RNA samples tested, the expression of BRP in small subpopulations of cells, or during certain developmental stages or physiological states in these tissues and others, cannot be ruled out.

For samples 1-3, a β-actin pre-developed TaqMan® assay (Applied Biosystems, Foster City, Calif.) was used to assess the efficiency of cDNA synthesis. Each sample was analyzed in m triplicate, using 50 ng input RNA per 25 μl reaction. The assay was done according to the manufacturer's specifications. The average C_{T} was 16 for all three cDNA samples, demonstrating efficient cDNA synthesis.

Samples 5-9 were tested for ARP levels for comparison to results obtained using the Origene panel. The ARP TaqMan® assay was used, with 25 ng input RNA per reaction. Results 15 showed the following relative order of expression: pancreas (C_{T}≅20)>pituitary (C_{T}≅22)>testis (C_{T}≅25)>kidney (C_{T}≅29)>Iiver (C_{T}≅31)>placenta (C_{T}≅33). These results were consistent with those reported in Example 4.

### Example 2

Isolation of a cDNA Clone Corresponding to Human BRP

The DNA sequence from Genbank accession number AL118555 (FIG. 4), and the PRIME software program (Wisconsin Package Version 10.1, Genetics Computer Group (GCG), Madison, Wis.) were used to design PCR primers for amplification of the predicted coding region of ARP/BRP, which is contained in two putative exons separated by an intron of approximately 5 kb. The sequences of the forward and reverse primers (called the ARP/BRPCDS primers) were 5′-CAGCATGAAGCTGGCATTCCTC-3′ (SEQ ID NO: 77) and 5′-GCCTCAGATGGTCTCACACTCC-3′, (SEQ ID NO: 48) respectively.

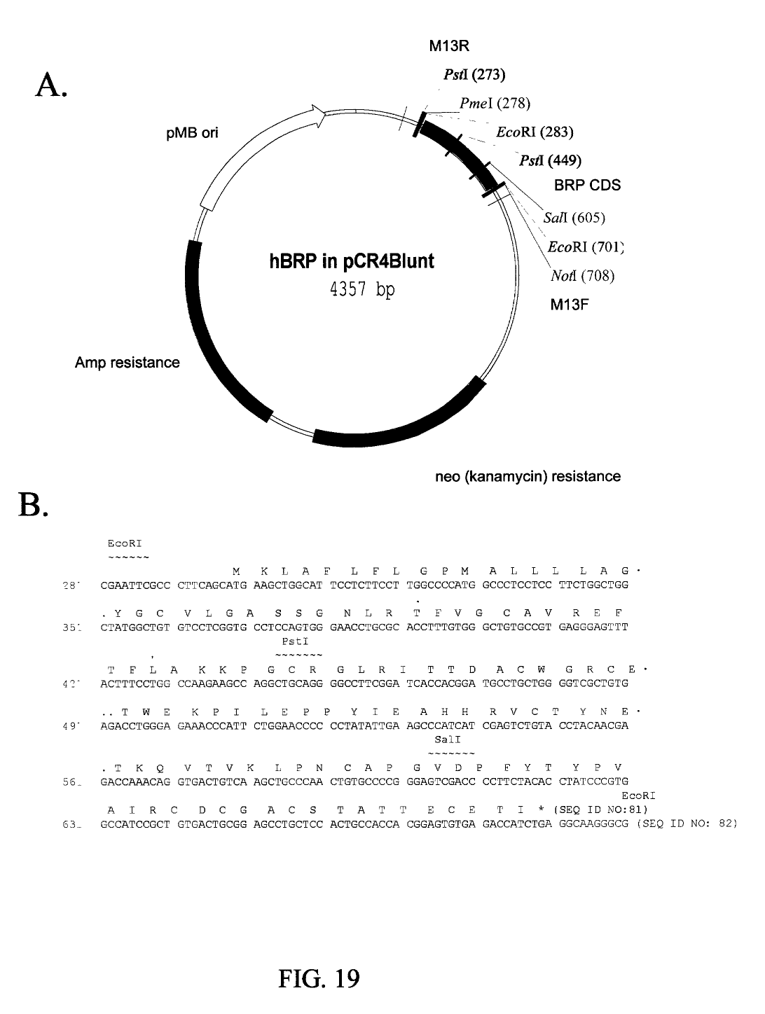

PCR reactions for cloning the human BRP protein coding region were done in a volume of 50 μl with the following components: 4 mM MgSO₄, 200 nM forward primer, 200 nM reverse primer, 200 μM each dCTP, dATP, dTTP, and dGTP, 1× Thermopol buffer (New England Biolabs, Beverly, Mass.), 0.5 unit Vent_{R} polymerase (New England Biolabs, Beverly, Mass.), and 2 μl pituitary cDNA (equivalent to cDNA prepared from 100 ng polyA+RNA as described in preceding section). The cycling conditions were 99.9° C. 2 min, followed by 40 cycles of 94° C. for 30 sec, 67° C. for 15 sec, and 75° C. for 30 sec. These conditions resulted in the detection of a faint band of approximately 400 base pairs (bp) on an agarose gel. The fragment was gel-purified and cloned using the Zero Blunt® TOPO® PCR cloning kit (Invitrogen, Carlsbad, Calif.). A plasmid with an EcoRI insert of approximately 400 bp was identified and called hBRP in pCR4Blunt (FIG. 19). Results of DNA sequencing confirmed that the identity of the 400 bp PCR fragment was BRP. The DNA sequence of the fragment was identical to that of SEQ ID NO: 1, with an open reading having an amino acid translation identical to SEQ ID NO: 2.

### Example 3

Construction of Plasmids for Expression of Human BRP Fusion Proteins in Mammalian Cells.

AP- BRP

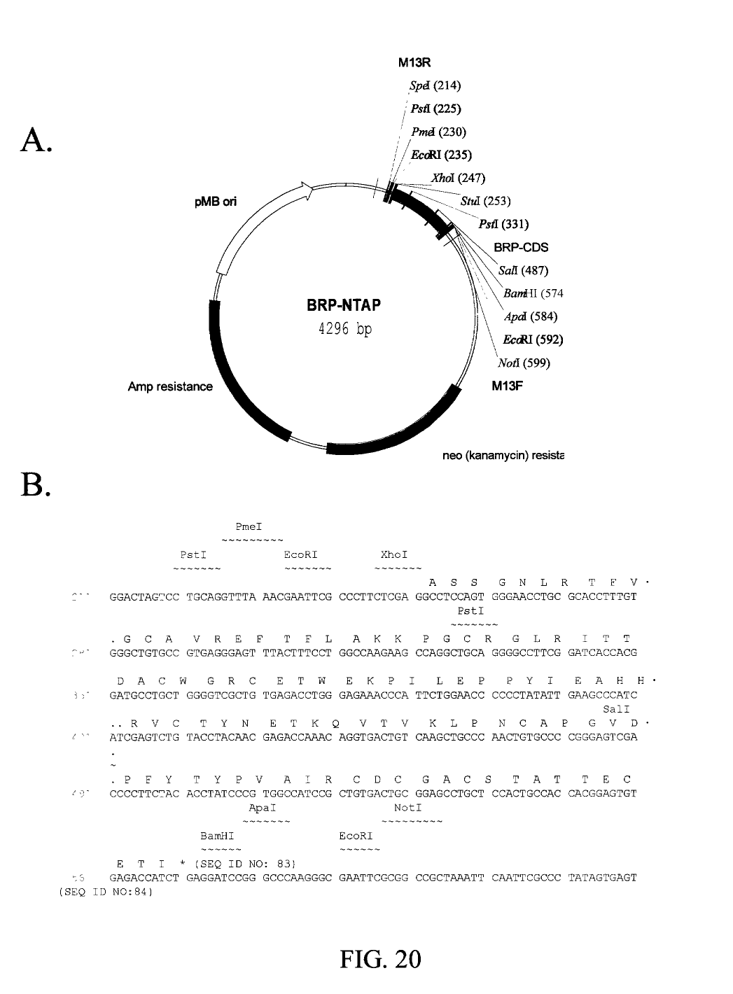

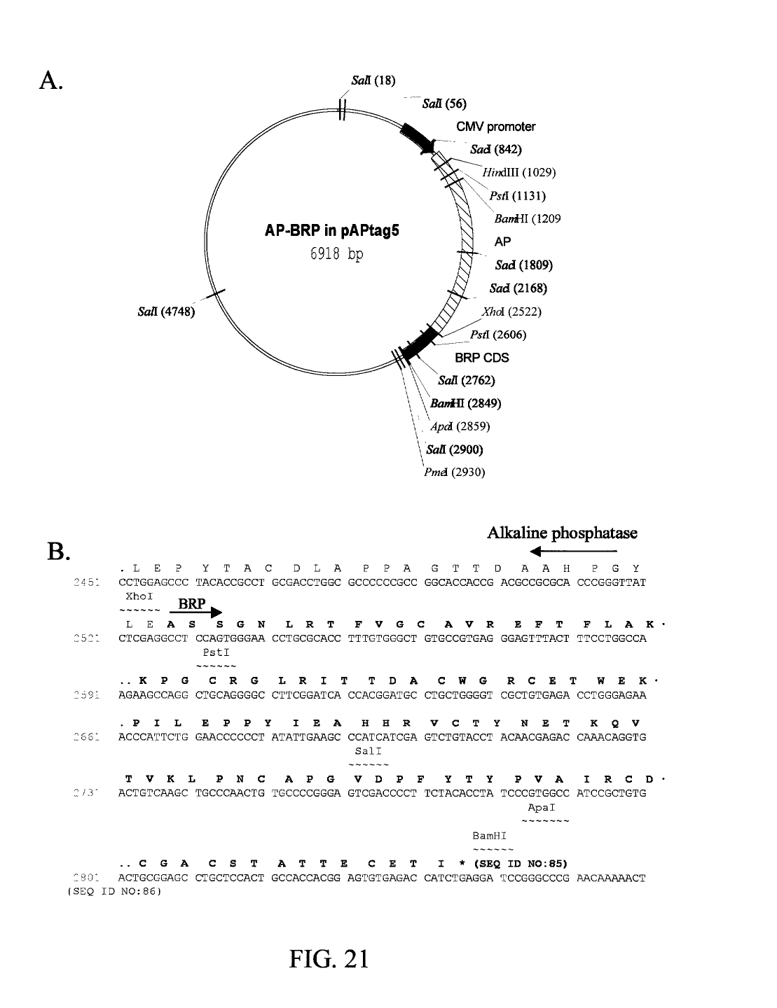

Fusion of the coding region for predicted mature human BRP(without the signal sequence) to the C-terminal region of alkaline phosphatase (AP) was done using the following primers 5′-CTCGAGGCCTCCAGTGGGAACCTGCGCAC-3′ (SEQ ID NO: 49)and 5′-GGGCCCGGATCCTCAGATGGTCTCACACTCC-3′ (SEQ ID NO: 50). PCR reaction conditions were as follows: 4 mM MgSO₄, 200 nM each primer, 200 μM each dCTP, dATP, dTTP, and dGTP, 1× Thermopol buffer (New England Biolabs, Beverly, Mass.), 1 unit Vent_{R} polymerase (New England Biolabs, Beverly, Mass.) and 100 ng ARP/BRPin pCR4Blunt plasmid DNA, in a 100 μl reaction volume. Cycling conditions were 99° C. 2 min, followed by 25 cycles of 94° C. 30 sec, 70° C. 30 sec. The resulting PCR fragment was gel purified and cloned using the Zero Blunt® TOPO® PCR cloning kit (Invitrogen, Carlsbad, Calif.). A plasmid called BRP-NTAP (FIG. 20) having the expected restriction endonuclease banding pattern was identified and sequenced. BRP-NTAP plasmid DNA was digested with XhoI and ApaI and the insert containing the BRPcoding region was gel-purified and inserted into XhoI and ApaI digested Aptag-5 vector DNA (GenHunter® Corporation, Nashville, Tenn.). This resulted in a plasmid engineered for the expression of a fusion protein consisting of secreted alkaline phosphatase at the N-terminus and BRPat the C-terminus (AP- BRPin Aptag-5, FIG. 21).

BRP-GFP

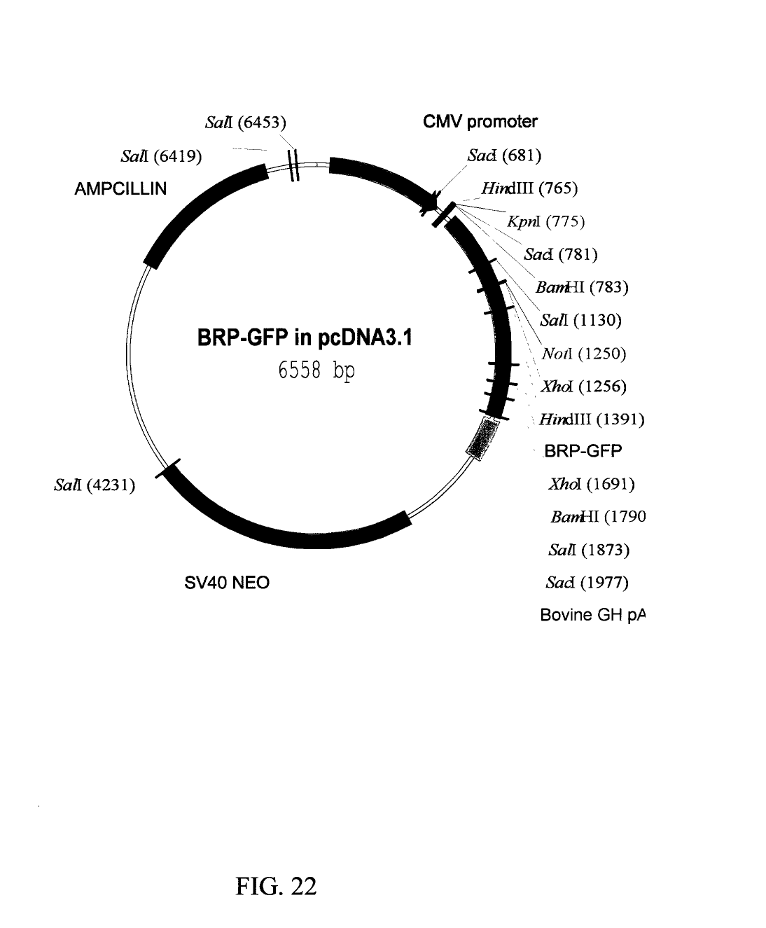

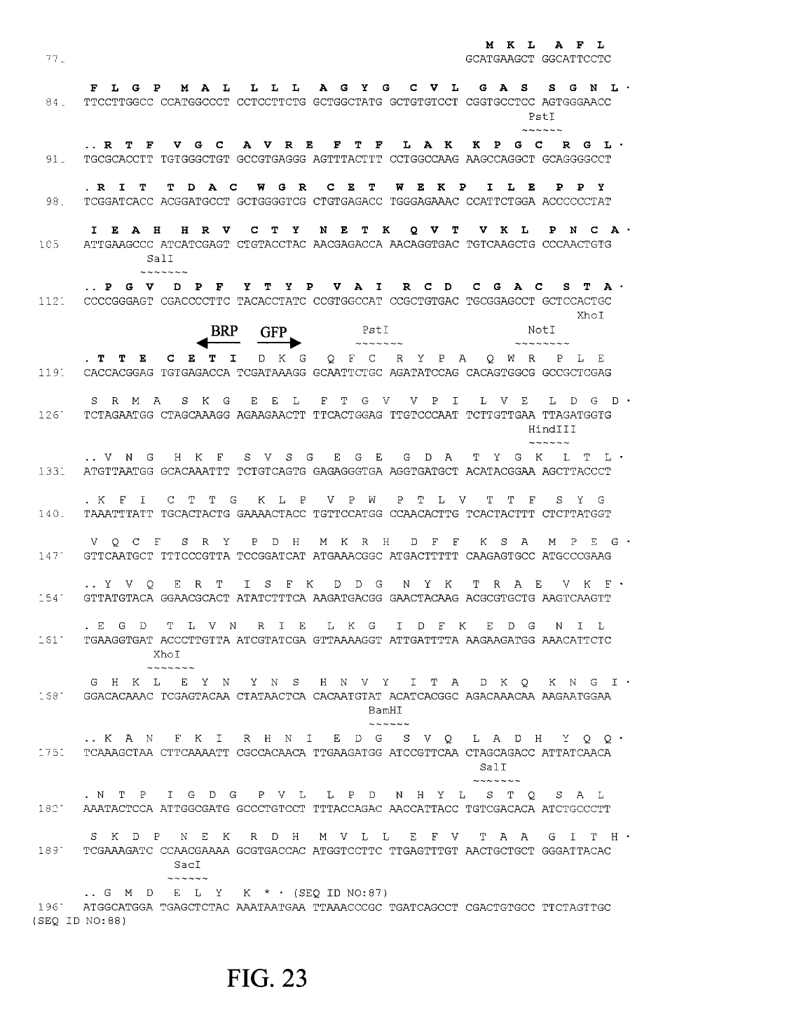

Fusion of green fluorescent protein (GFP) to the C-terminus of human BRP was done using the PCR primers 5′-GCTAGCATGAAGCTGGCATTCCTC-3′ (SEQ ID NO: 51)and 5′-TATCGATGGTCTCACACTCCGTG-3′(SEQ ID NO: 52). PCR reaction conditions were as follows: 4 mM MgSO₄, 200 nM each primer, 200 μM each dCTP, dATP, dTTP, and dGTP, 1× Thermopol buffer (New England Biolabs, Beverly, Mass.), 1 unit Vent_{R} polymerase (New England Biolabs, Beverly, Mass.) and 50 ng hBRP in pCR4Blunt plasmid DNA, in a 50 μl reaction volume. Twelve identical 50 μl reactions were prepared to test a 12-point gradient of annealing temperatures ranging from 49° C. to 69° C. Cycling conditions were 99° C. 5 min followed by 25 cycles of 94° C. 30 sec, 49-69.1° C. 15 sec, and 75° C. 30 sec. PCR product from the reactions with annealing temperatures of 63-68° C. were pooled and gel-purified. In order to add dA residues to the termini, the purified fragment was treated with 2.5 units of Taq DNA polymerase (Life Technologies, Rockville, Md.) in a 100 μl reaction volume with 1× PCR buffer minus Mg (Life Technologies, Rockville, Md.), 2 mM MgCl₂, 200 μM each dCTP, DATP, dTTP, and dGTP. After incubation at 72° C. for 15 min, the fragment was purified using the Wizard® PCR Preps DNA purification system (Promega Corporation, Madison, Wis.). The dA-tailed BRPPCR fragment was inserted into the vector provided in the CT-GFP Fusion TOPO® TA Expression kit (Invitrogen, Carlsbad, Calif.) using the protocol specified by the manufacturer. An expression vector for the expression of a fusion protein consisting of human BRP at the N-terminus, and Cycle 3 GFP at the C-terminus (BRP-GFP in pcDNA3.1, FIG. 22) was obtained. The structure of the fusion construct was confirmed by DNA sequencing (FIG. 23).

FLAG- BRP

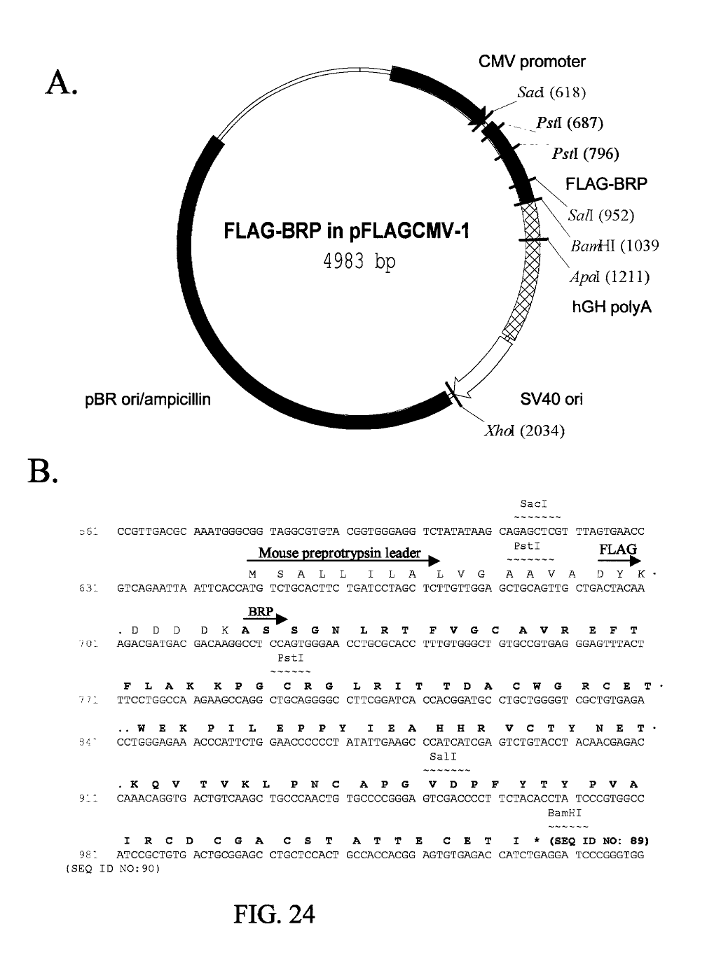

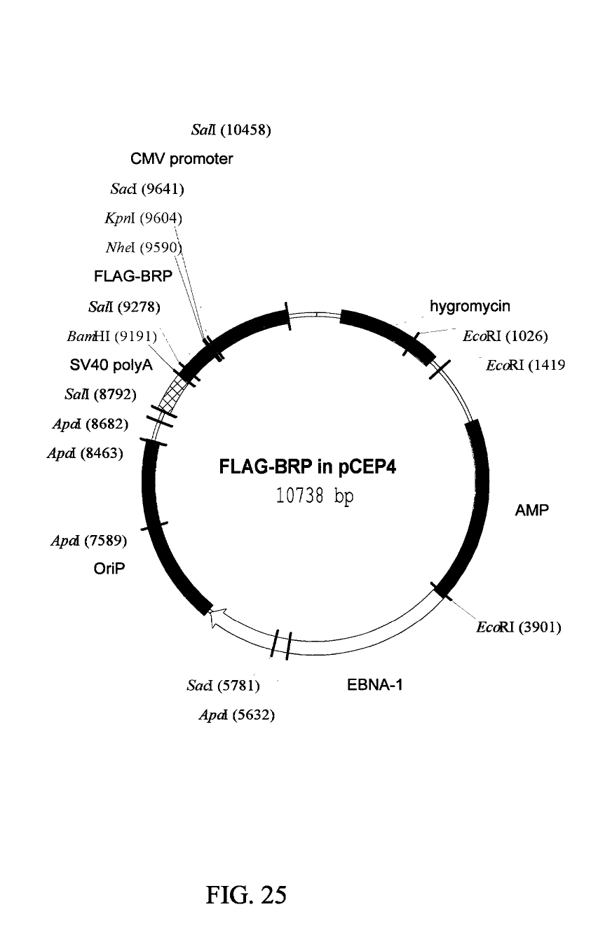

In order to engineer an vector for the expression of BRP with an N-terminal FLAG tag, the BRP-NTAP plasmid was digested with EcoRI and BamHI and the approximately 400 bp human BRP insert was gel-purified. The purified DNA fragment was inserted into pFLAG-CMV-1 (Sigma Chemical Co., St. Louis, Mo.) digested with EcoRI and BamHI. The resulting plasmid construct (pFLAG-CMV- BRP-RI-BAM) was digested with PstI and the 4.9 kilobase pair (kb) vector fragment was gel-purified to remove the 100 bp PstI fragment. BRP-NTAP was digested with SpeI and StuI, the 4.3 kb vector DNA fragment was gel-purified and then ligated to complementary oligonucleotides having the sequences 5′-CTAGTCTCGAGGCTGCAGTTGCTGACTACAAAGACGATGACGACAAGG-3′ (SEQ ID NO: 53) and 5′-CCTTGTCGTCATCGTCTTTGTAGTCAGCAACTGCAGCCTCGAGA-3′(SEQ ID NO: 54). After cloning, a plasmid construct with the correct sequence was identified and was digested with PstI. The 100 bp PstI fragment was gel-purified and inserted into the 4.9 kb PstI vector fragment from pFLAG-CMV- BRP-RI-BAM (above). Constructs with the PstI insert in the correct orientation were identified and sequenced to confirm that a FLAG-BRPfusion was encoded in frame with the mouse preprotrypsin signal sequence of pFLAG-CMV-1. This expression vector plasmid was called FLAG- BRPin pFLAG-CMV-1 (FIG. 24). The FLAG- BRPinsert was PCR amplified using the primers 5′-TTTGCTAGCACCATGTCTGCACTTCTG-3′ (SEQ ID NO: 55)and 5′-TTTGGATCCTCAGATGGTCTCACACTC-3′(SEQ ID NO: 56). PCR reaction conditions were as follows: 4 mM MgSO₄, 200 nM each primer, 200 μM each dCTP, dATP, dTTP, and dGTP, 1 × Thermopol buffer (New England Biolabs, Beverly, Mass.), 1 unit Vent_{R} polymerase (New England Biolabs, Beverly, Mass.) and 50 ng FLAG- BRPin pFLAG-CMV-1 plasmid DNA, in a 50 μl reaction volume. Twelve identical 50 μl reactions were prepared to test a 12-point gradient of annealing temperatures ranging from 65° C. to 75° C. Cycling conditions were 99° C. 5 min followed by 30 cycles of 94° C. 30 sec, 65-75° C. 15 sec, and 75° C. 30 sec. PCR product from the reactions with annealing temperatures of 65-71° C. were pooled and gel-purified. The fragment was digested with NheI and BamHI and then ligated to NheI and BamHI digested pCEP4 plasmid DNA (Invitrogen, Carlsbad, Calif.) to give the primate cell expression vector plasmid FLAG- BRPin pCEP4 (FIG. 25). DNA sequence analysis was used to confirm the correct sequence for coding the FLAG- BRP fusion protein.

HIS-ARP

A two stage PCR amplification was done to obtain a fusion protein consisting of (1) the mouse preprotrypsin signal peptide, (2) a six histidine-one glycine tag (6Hisg) and (3) an enterokinase cleavage site (EK) at the N-terminus of ARP. In the first stage, 11.73 ng FLAG-ARP-Phe in pCEP4 plasmid DNA was used as the template for primers 5′-CTCTTGTTGGAGCTGCAGTTGCTCATCATCACCATCACCATGGTGACGATGACGATAAGCAGGAGGCAG-3′ (SEQ ID NO: 103) and 5′-TTTGGATCCGTCGACTAGTAGCGAGAGAGGCGACACATG-3′(SEQ ID NO: 104). PCR was done in 9 identical 53 μl reactions containing 1 unit Vent_{R} polymerase (New England Biolabs, Beverly, Mass.), 4 mM MgSO₄, 420 nM each primer, 200 μM each dCTP, DATP, dTTP, and dGTP, and 1× Thermopol buffer (New England Biolabs, Beverly, Mass.). Cycling conditions were 99° C. 5 min followed by 30 cycles of 94° C. 30 sec, 68° C. 30 sec, and 75° C. 30 sec. Following PCR, 1 μl of the first stage reaction was transferred to a new tube for the second stage reaction with the primers and 5′-TTTGCTAGCGTCGACCATGTCTGCACTTCTGATCCTAGCTCTTGTTGGAGCTGCAGTTGCTCATC-3′(SEQ ID NO: 105) and 5′-TTTGGATCCGTCGACTAGTAGCGAGAGAGGCGACACATG-3′ (SEQ ID NO: 106).

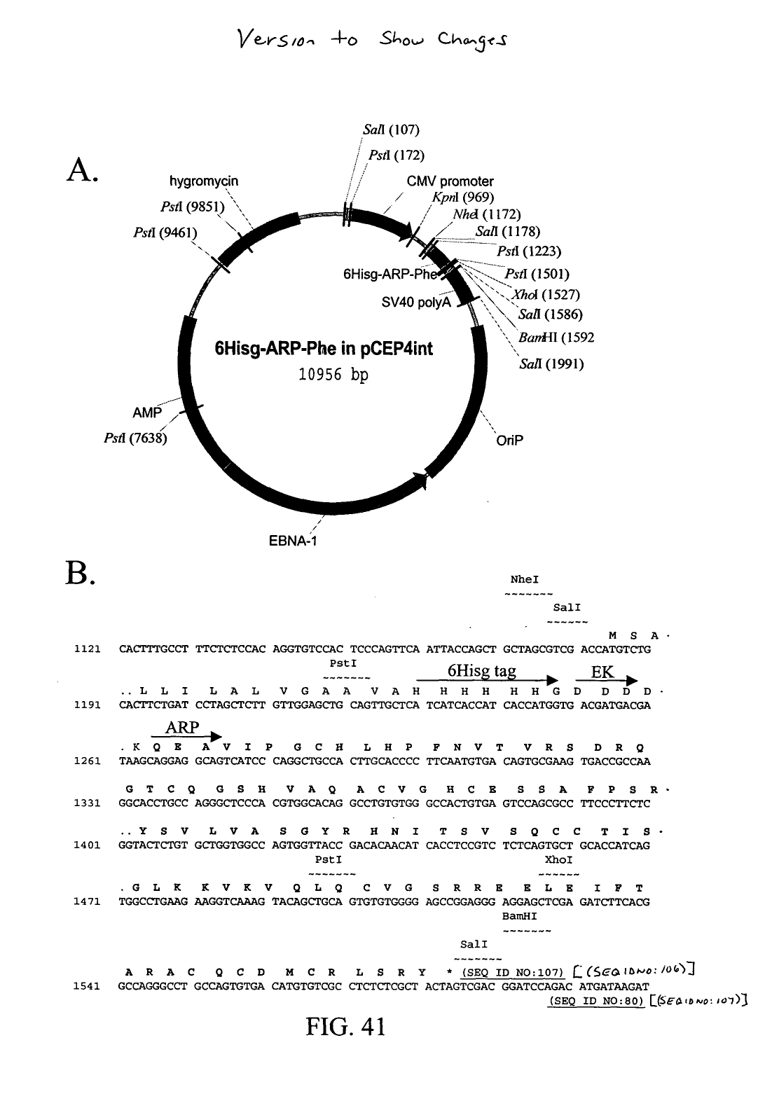

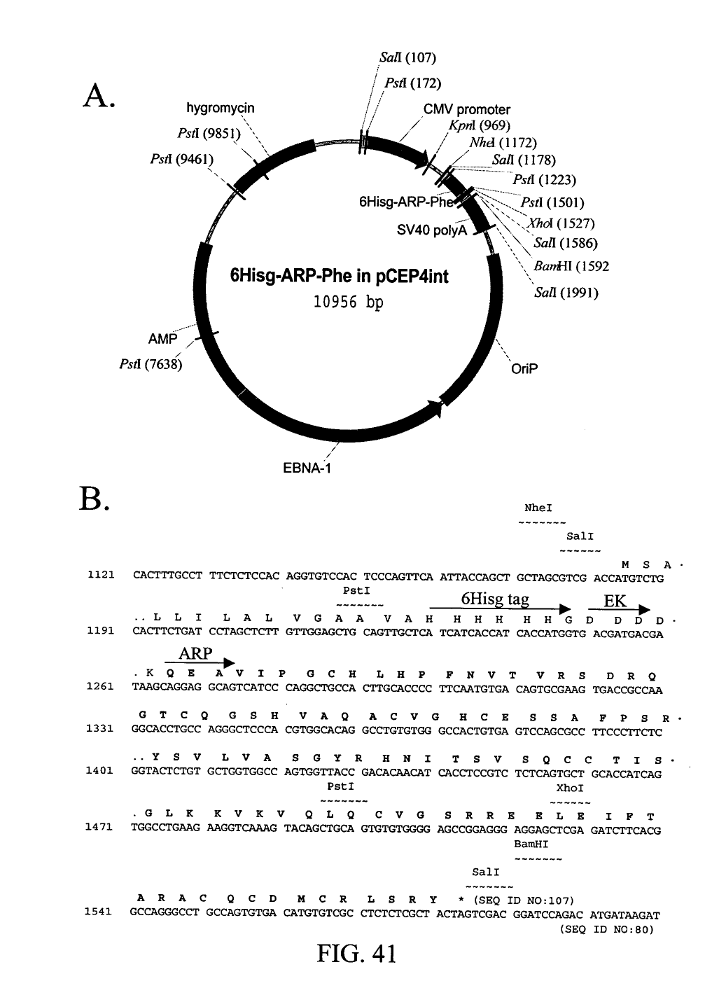

The volume of the reaction was 120 μl, with all other reaction conditions the same as for the first stage. Aliquots, 12 μl each, of the reaction mix were used to test a nine-point gradient of annealing temperatures ranging from 65° C. to 76° C. Cycling conditions were 99° C. 5 min followed by 30 cycles of 94° C. 30 sec, 65-76° C. 30 sec, and 75° C. 30 a fragment of the expected size that was gel-purified and digested with NheI and BamHI, concentrated using the Wizard® PCR Preps DNA purification system (Promega), then inserted into pCEP4int digested with NheI and BamHI. A plasmid with a correctly sized insert was identified and called 6Hisg-ARP-Phe in pCEP4int (abbreviated to His-ARP). The configuration of the fusion protein was confirmed by DNA sequencing. A diagram of the plasmid is shown in FIG. 41A with the DNA sequence and amino acid translation of the fusion protein shown in FIG. 41B.

### Example 4

Detection of Human ARP Transcripts in RNA Extracted from Human Tissues

The human ARP forward and reverse primers used in the TaqMan® assay were 5′-AGGAGGCAGTCATCCCAGG-3′ (SEQ ID NO: 57)and 5′-TGCCTTGGCGGTCACTTC-3′(SEQ ID NO: 58), respectively. The probe for ARP was 5′-6FAM-TGCCACTTGCACCCCTTCAATGTG-TAMRA-3′(SEQ ID NO: 59). The fluorescent probe spanned the predicted junction of the first two coding exons for ARP, thus the assay is intended to specifically detect the spliced mRNA transcript.

A Human Rapid-Scan™ Expression Panel (OriGene Technologies, Inc., Rockville, Md.) was used to provide cDNA templates for the ARP TaqMan® assay. The panel contained cDNA from 24 tissues serially diluted from 1000×, 100×, 10×, and 1×. The lowest concentration, 1×, was approximately 1 pg cDNA. The 1000× and 100× dilutions were used with for the ARP TaqMan® assay. The wells from duplicate panels containing the 1× cDNA concentration were used for the human β-actin pre-developed assay reagent kit (Applied Biosystems, Foster City, Calif.).

The panel did not include cDNA from the pituitary, therefore the pituitary cDNA from Example 1 was used. The β-actin pre-developed TaqMan® assay and dilutions of the pituitary cDNA were used to determine the approximate amounts equal to the 1000×, 100×, and 1× cDNA dilutions on the Origene panel. Results showed that cDNA prepared from 5 pg pituitary poly(A)+RNA was equivalent to the 1× cDNA concentration, giving CTS of approximately 31. Therefore cDNA from 5 ng and 0.5 ng of pituitary poly(A)+RNA were considered equivalent to the Origene panel 1000× and 100× cDNAs, respectively.

Results are shown in Table 6.

TABLE 6
Determination of relative amount of human ARP mRNA in
tissues using TaqMan ® real-time quantitative PCR.

Relative
amounts of ARP

| | | |
|---|---|---|
| ARP Ct | β-actin CT | mRNA, |

| | 1000 pg | 100 pg | 1 pg | 1 pg | normalized to |
|---|---|---|---|---|---|
| Tissue | cDNA | cDNA | cDNA | cDNA | β-actin¹ |
| Pancreas | 23.3 | 25.9 | 32.2 | 31.2 | 100.00 |
| Pituitary | 26.1 | 29.0 | 31.0 | 30.6 | 6.39 |
| Testis | 27.9 | 30.4 | 30.1 | 30.2 | 1.45 |
| Kidney | 30.5 | 33.7 | 31.1 | 30.4 | 0.26 |
| Ovary | 31.8 | 34.9 | 31.9 | 31.6 | 0.23 |
| Prostate | 31.1 | 33.9 | 31.0 | 30.4 | 0.20 |
| Skin | 31.6 | 36.1 | 31.0 | 31.1 | 0.11 |
| Salivary | 31.7 | 34.6 | 30.3 | 30.4 | 0.10 |
| Adrenal | 30.6 | 33.9 | 29.4 | 29.2 | 0.09 |
| Gland | | | | | |
| Stomach | 32.1 | 34.8 | 30.4 | 30.1 | 0.07 |
| Brain | 33.2 | 35.9 | 31.1 | 31.0 | 0.06 |
| Fetal Liver | 31.8 | 35.2 | 30.0 | 29.2 | 0.05 |
| Liver | 33.6 | 35.6 | 30.4 | 29.9 | 0.04 |
| Thyroid | 35.0 | 36.3 | 30.4 | 30.0 | — |
| Lung | 35.0 | 37.2 | 31.1 | 30.6 | — |
| Fetal Brain | 34.8 | 37.1 | 30.6 | 30.1 | — |
| Uterus | 35.9 | 39.3 | 31.1 | 31.2 | — |
| Spleen | 37.2 | 38.7 | 31.1 | 31.0 | — |
| PBL | 36.7 | 40.0 | 31.4 | 31.2 | — |
| Colon | 40.0 | 40.0 | 32.2 | 31.9 | — |
| Small | 35.5 | 40.0 | 30.6 | 30.3 | — |
| Intestine | | | | | |
| Bone | 40.0 | 40.0 | 31.6 | 31.2 | — |
| Marrow | | | | | |
| Heart | 35.4 | 40.0 | 31.3 | 28.3 | — |
| Muscle | 36.6 | 40.0 | 30.3 | 30.0 | — |
| Placenta | 40.0 | 40.0 | 29.7 | 29.6 | — |
| ¹Relative amounts of the target (ARP or β-actin) were determined by assuming a 100% efficiency of the PCR so that each cycle difference was equivalent to a 2-fold difference in target cDNA. For example, the fold difference of cDNA target in tissue A vs. tissue B = 2^{[Ct(tissue A) minus Ct(tissue B)]}. The 1000X and 100X ARP values were averaged and compared to averaged | | | | | |
| # β-actin values to obtain normalized relative values. A Ct of 35 or greater was considered negative. | | | | | |

The results show that of the tissue samples examined, pancreas, pituitary, and testis tissue contain the highest levels of ARP. The remaining tissues either had lower or undetectable levels of ARP mRNA.

### Example 5

Detection of ARP mRNA in Rat Tissue

Rat RNA was purchased from Clontech (Palo Alto, Calif.) or prepared from organs flash frozen in liquid nitrogen and stored at −80° C. Tissues up to 40 mg in weight were crushed by a hand held pestle (Kontes, Vineland, N.J.) for 45 sec in RLT buffer (Rneasy® kits Qiagen Inc. Valencia, Calif.), followed by homogenization with 10 passes through a 21 gauge needle. A Brinkman Polytron was used to homogenize the larger tissues in either RLT buffer or Trizol® (Life Technologies, Rockville, Md.). RNA was purified according to the protocol supplied by the manufacturer of the homogenization buffer used. Preparation of cDNA was done as described in Example 1.

The rat ARP forward and reverse primers used in the TaqMan® assay were 5′-AGGCAGCCGTCCCAATC-3′ (SEQ ID NO: 60) and 5′- GATCACTTCGCACTGTCACGTT-3′ (SEQ ID NO: 61), respectively. The probe for rat ARP was 5′-6FAM-CAGGCTGCCACTTGCACCCCTT-TAMRA-3′ (SEQ ID NO: 62). The fluorescent probe spanned the predicted junction of the first two coding exons for ARP, thus the assay is intended to specifically detect the spliced mRNA transcript.

ARP cDNA was detectable by TaqMan® PCR in the rat pituitary, ovary, testis, eye and rat pituitary adenoma cell line RC-4B/C (data not shown). Results of assays done on total RNA extracted from rat pituitary tissue taken from 76 day mature female animals in proestrus, estrus, and diestrus (as determined by vaginal smear), suggested that rat ARP mRNA is regulated during the estrus cycle and thus may have a role in reproduction. The regulation appears to be the opposite of that of FSHβ mRNA, in that ARP mRNA levels decrease during estrus, whereas FSHβ mRNA levels increase (Table 7).

TABLE 7
ARP and FSHβ mRNA levels in the pituitary of mature female
rats at proestrus, estrus, and diestrus.

| | Number | ARP mRNA levels | FSHβ mRNA levels |
|---|---|---|---|
| Animal Group | per group | (relative)¹ | (relative)² |
| Proestrus | 3 | 27.18 ± 4.98 | 5.05 ± 1.91 |
| Estrus | 5 | 17.95 ± 5.66 | 7.35 ± 2.56 |
| Diestrus | 5 | 32.96 ± 11.59 | 4.02 ± 1.42 |

¹ARP mRNA values were determined relative to a standard curve generated with rat pituitary cDNA, and were normalized to 18S RNA
²FSHβ mRNA values were determined relative to a standard curve generated with a rat FSHβ DNA fragment produced by PCR, and were normalized to 18S RNA.

### Example 6

Isolation of cDNA Clone Corresponding to Human ARP

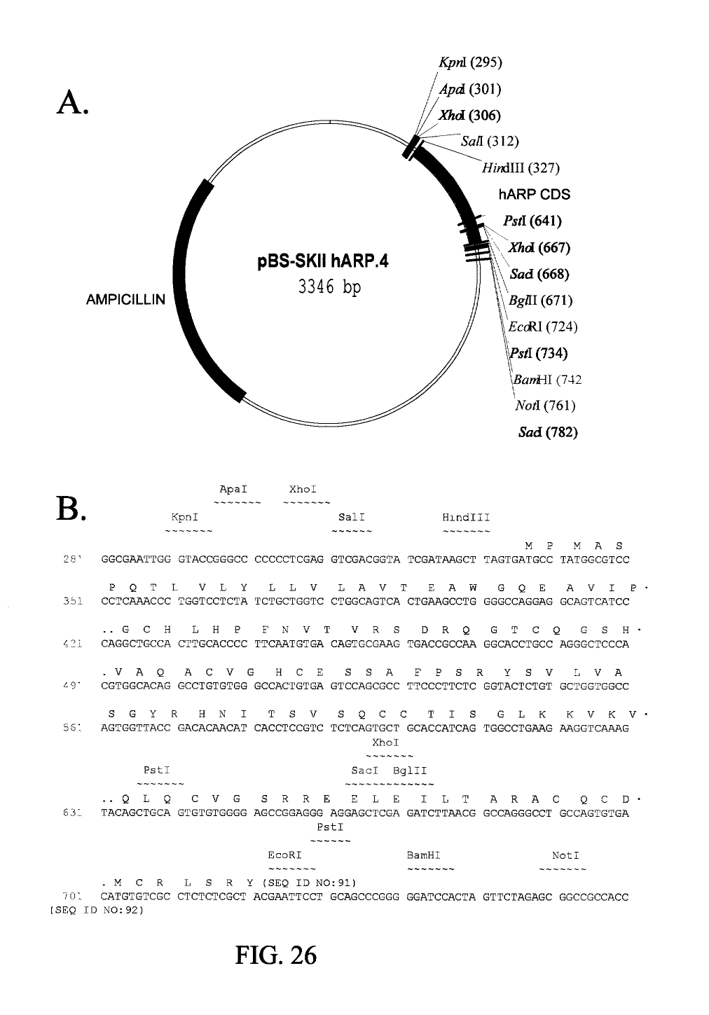

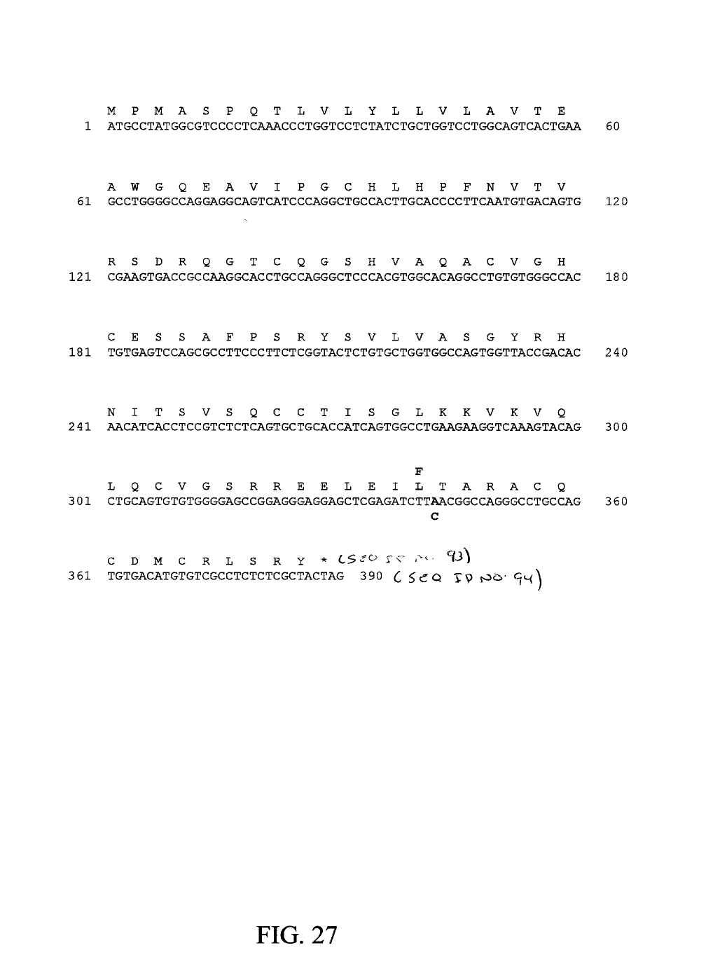

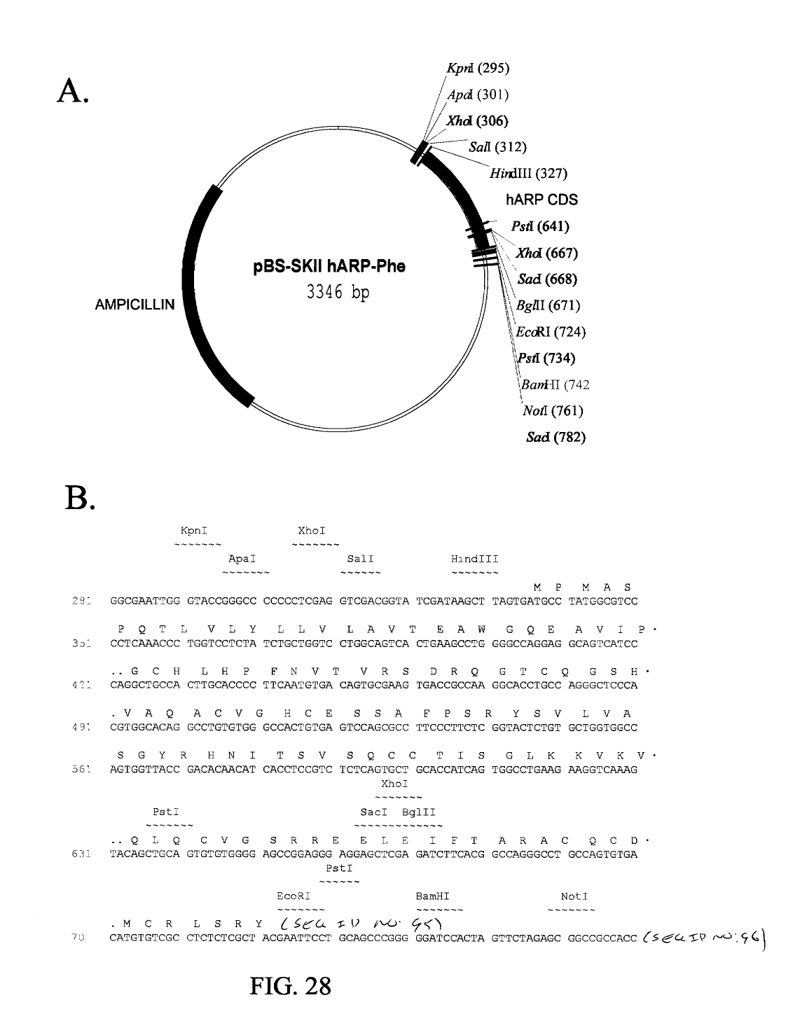

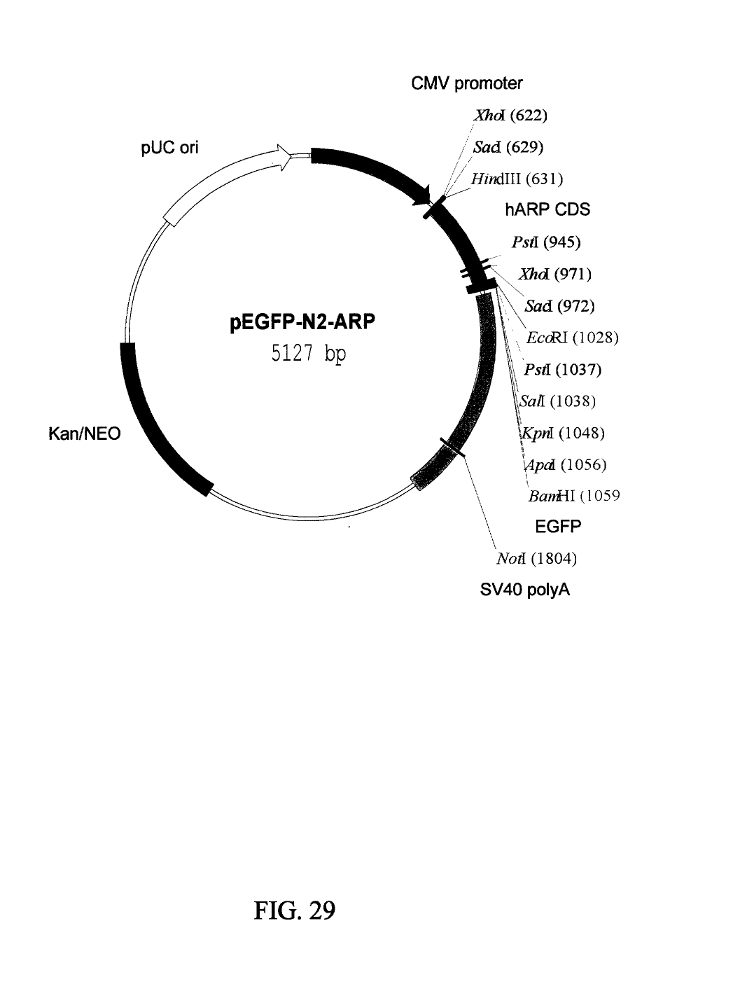

DNA purified from the IMAGE 2338950 clone (Research Genetics, Huntsville, Ala.) was used as a template for PCR with the following forward and reverse PCR primers: 5′-TTTTAAGCTTAGTGATGCCTATGGCGTCCCC-3′ (SEQ ID NO: 63)and 5′-TTTTGAATTCGTAGCGAGAGAGGCG-3 (SEQ ID NO: 64)′ (no stop codon), respectively. PCR was done in a reaction volume of 100 μl with 4 units Vent_{R} polymerase (New England Biolabs, Beverly, Mass.), 1 μM each of the forward and reverse primers, 2 mM MgSO₄, 250 μM each dCTP, dATP, dTTP, and dGTP, 1× Thermopol buffer (New England Biolabs, Beverly, Mass.), and 1 μl of IMAGE 2338950 plasmid DNA. Cycling conditions were 30 cycles of 94° C. 1 min, 56° C. 35 sec, 72° C. 1 min. The approximately 400 bp fragment obtained from the PCR was gel-purified, digested with HindII and EcoRI, then ligated into HindII and EcoRI digested pBluescriptSKII vector DNA (Sratagene, La Jolla, Calif.). The resulting plasmid, pBSSKII hARP.4 (26A) was subjected to DNA sequence analysis to confirm the identity of the insert 109 fragment as ARP. The sequence of the human ARP protein coding region, (FIG. 26B) was identical to SEQ ID NO: 17. Interestingly, the DNA sequence shown in SEQ ID NO: 23 has a single nucleotide difference when compared to both the cloned ARP insert in pBSSKII hARP.4 and SEQ ID NO: 17. This difference (ARC) is illustrated in FIG. 29, and results in a Leu to Phe change in the ARP amino acid sequence at residue 114. A search of the EST database revealed that the ARP-Phe form is predominant and thus the ARP Leu form is a possible polymorphic variant that may be less common in the population. In order to obtain a cDNA clone that encoded the ARP-Phe protein, the A residue corresponding to the polymorphism was mutated to C in pBSSKII hARP.4 so that the ARP-Phe form was encoded. This was done by using pBSSKII hARP.4 as a template for PCR with a primer corresponding to the T3 promoter sequence in pBluescript SKII and 5′-TTTGAGATCTTCACGGCCAGGG-3′ (SEQ ID NO: 65). Reaction conditions and cycling parameters were similar to those described for above for cloning the complete ARP coding region. The resulting PCR fragment was gel purified and cloned using the Zero Blunt® TOPO® RCR cloning kit (Invitrogen, Carlsbad, Calif.). DNA sequence analysis confirmed the presence of the mutation in a plasmid called pCRblunt Phe. Purified DNA from pCRblunt Phe was digested with BgII and NotI. The ARP fragment containing the Phe mutation was gel-purified and inserted into pBSSKII hARP.4 that had been digested with BglII and NotI, and purified from the ARP-Leu fragment. A plasmid with an insert of the expected size, called pBSSKII hARP-Phe (FIG. 28A), was identified and was shown by DNA sequence analysis to have an open reading frame that correctly encoded the ARP-Phe variant (FIG. 28B).

### Example 7

Construction of Plasmids for Expression of Human ARP Fusion Proteins in Mammalian Cells.

GFP-ARP

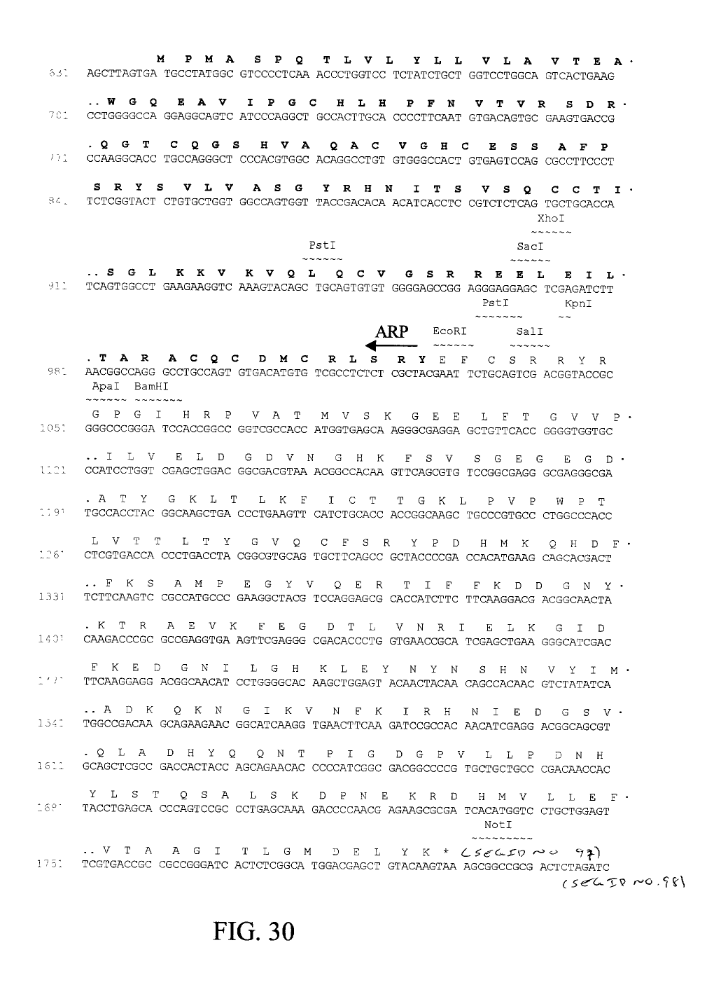

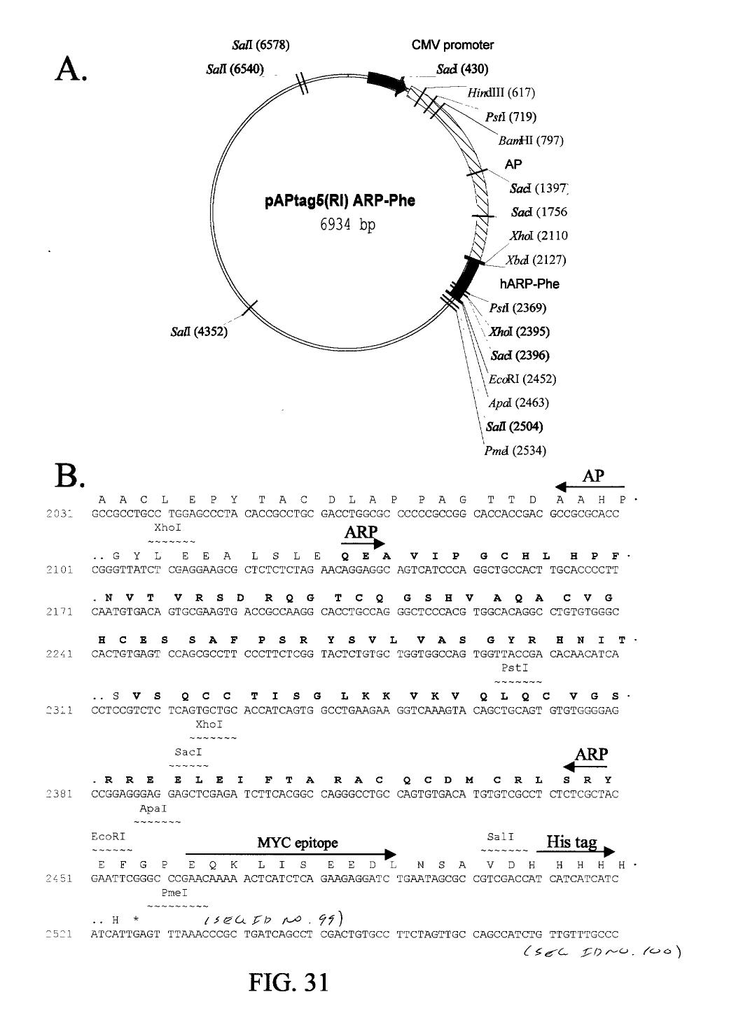

The HindII-EcoRI insert from pBSSKII ARP.4 was subcloned into HindII and EcoRI digested pEGFP-N2 (Clontech, Palo Alto, Calif.). Clones with the correct restriction endocuclease banding patterns were identified. One was selected for further studies and called pEGFP-N2-ARP, or ARP-GFP (FIG. 29). The DNA sequence of the fusion protein ORF with the corresponding amino acid translation is shown in FIG. 30.

AP-ARP

To facilitate subcloning, a oligonucleotide adapter cassette (5′-CTAGAGGAATTCGGGCC-3′ (SEQ ID NO: 66) and 5′-CGAATTCCT-3′ (SEQ ID NO: 67)) was used to insert an EcoRI site between the XbaI and ApaI sites in the polylinker of pAPtag5 and create the vector pAPtag5(RI). To obtain an ARP coding fragment without the signal peptide, PCR amplification was done with the primers 5′-TTTTCTAGAACAGGAGGCAGTCATCCCAGGC-3′ (SEQ ID NO: 68) and 5′-TTTTGAATTCCTAGTAGCGAGAGAGGCG-3′ (SEQ ID NO: 69) and pBSSKII ARP.4 as the template. The 340 bp PCR product was digested with XbaI and EcoRI, purified, and inserted into pAPtag5(RI) that had been digested with XbaI and EcoRI. This produced a vector called pAPtag5(RI)-ARP-Leu that is suitable for the expression of the ARP-Leu variant tagged at the N-terminus with secreted alkaline phosphatase. To construct a vector for the expression of AP-ARP-Phe, the following fragments were isolated and combined in a ligation reaction to give the plasmid pAPtag5(RI)-ARP-Phe (FIG. 3 1A): the 6.6 kb XbaI-EcoRI fragment from pAPtag5(RI), the 240 bp XbaI-PstI fragment from pAPtag5(RI)-ARP-Leu, and the 83 bp PstI-EcoRI fragment from pBS SKII ARP-Phe. The DNA sequence of the AP-ARP junction region was determined and shown to encode the expected open reading frame.

FLAG-ARP

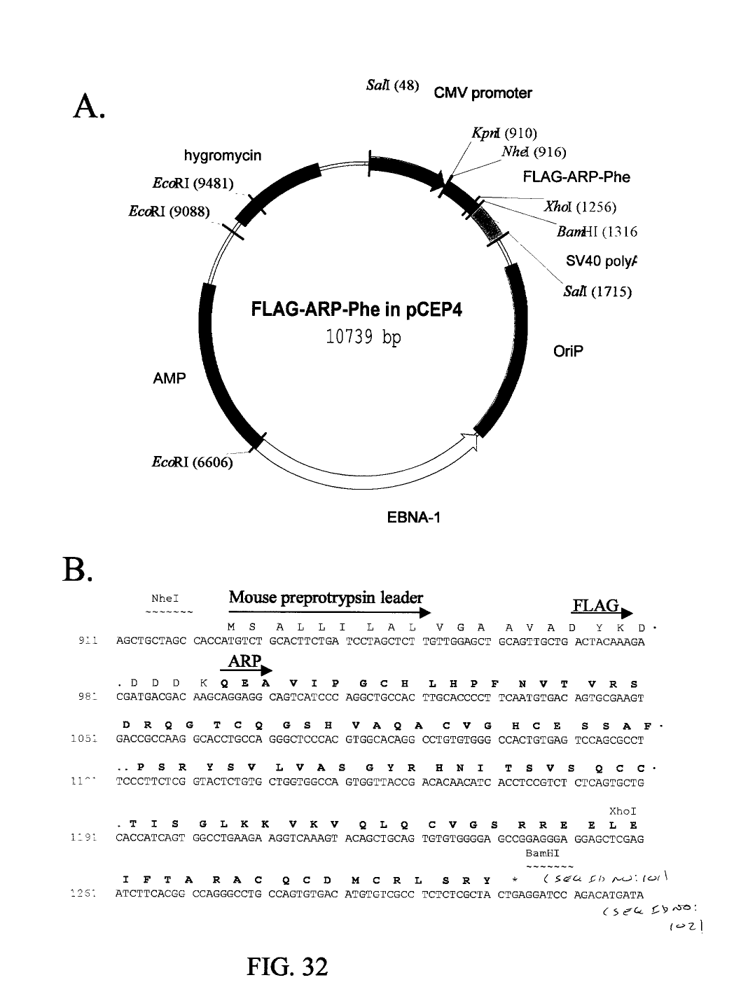

A two stage PCR amplification was done to obtain a fusion protein consisting of FLAG at the N-terminus of ARP with the mouse preprotrypsin signal peptide upstream of the FLAG tag. In the first stage, pBSSKII ARP-Phe (1 μl plasmid DNA) was used as the template for primers 5′-AGTTGCTGACTACAAAGACGATGACGACAAGCAGGAGGCAGTCATCCCAGGC-3′ (SEQ ID NO: 70) and 5′-CCCGTTTAAACGGATCCTCAGTAGCGAGAGAGGCGACACATG-3′ (SEQ ID NO: 71). PCR was done in a 50 μl reaction with 1 unit Vent_{R} polymerase (New England Biolabs, Beverly, Mass.), 4 mM MgSO₄, 100 nM each primer, 200 μM each dCTP, dATP, dTTP, and dGTP, and IX Thermopol buffer (New England Biolabs, Beverly, Mass.). Sixteen cycles of 96° .C 30 sec, 70° C. 30 sec were used for amplification. Following PCR, 10 μl of the first stage reaction mix was transferred to a new tube for the second stage reaction with the primers 5′-TTTGCTAGCCACCATGTCTGCACTTCTGATCCTAGCTCTTGTTGGAGCTGCAGTTGCTGACTACAAAGACGATG-3′ (SEQ ID NO: 72) and 5′-CCCGTTTAAACGGATCCTCAGTAGCGAGAGAGGCGACACATG-3′(SEQ ID NO: 73). The volume of the reaction was 100 μl, with all other reaction and cycling conditions the same as for the first stage. The PCR fragment produced from the second stage of PCR was digested with NheI and BamHI, then inserted into pCEP4 digested with NheI and BamHI. A plasmid with a correctly sized insert was identified and called FLAG-ARP-Phe in pCEP4 (FIG. 32).

FLAG-ARP-int

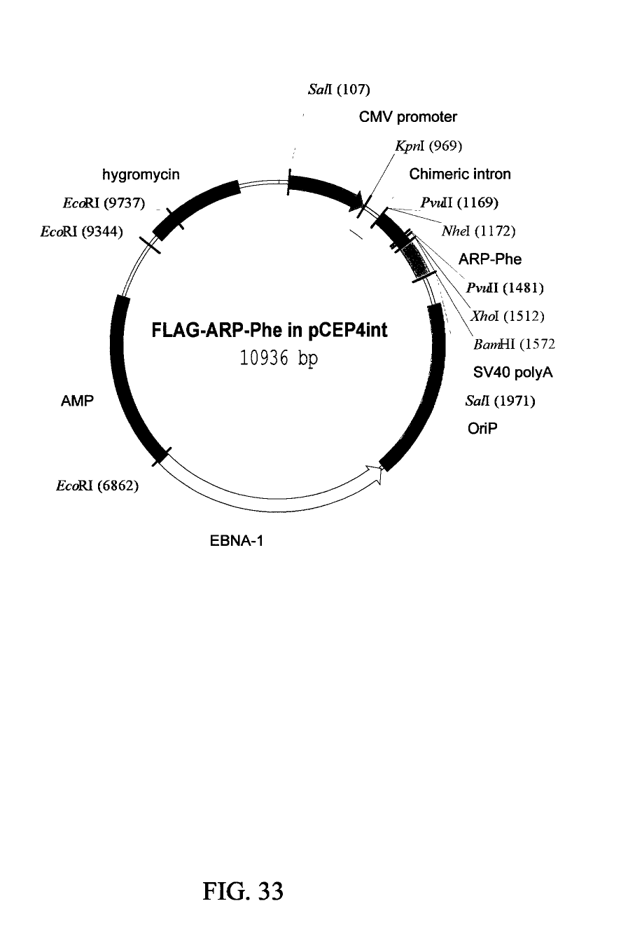

The intron-exon structure of the ARP gene is similar to that of its relative, the glycoprotein alpha subunit. Since the glycoprotein alpha subunit requires an intron or a genomic fragment for efficient expression in mammalian cells (U.S. Ser. No. 5,674,711), it is possible that ARP also has this requirement. To test this, a FLAG-ARP expression construct was engineered with an intron in the 5′ untranslated region of the mRNA. The chimeric intron from pCIneo (Promega Corporation, Madison, Wis.) was PCR amplified with primers designed to add a 5′ KpnI site (5′-GGTACCAAGGTAGCCTTGCAGAAGTT-3′ (SEQ ID NO: 74) and a 3′PvuII site (5′-CAGCTGGTAATTGAACTGGGAGTGGA-3′ (SEQ ID NO: 75)). The reaction mix included 10 ng pCIneo DNA, 1× Pfu buffer (Stratagene, La Jolla, Calif.), 1 μM each PCR primer, 200 nM each dATP, dGTP, dTTP, and dCTP, 3.2 mM MgCl₂, and 0.5 μl Pfu Turbo polymerase (Stratagene, La Jolla, Calif.) in a total volume of 25 μl. The cycling conditions were 95° C. 1 min followed by 20 cycles of 95° C. 30 sec, 55° C. 30 sec, 72° C. 1 min. After cycling, incubated at 72° C. for 10 min, then cooled to 4° C. The approximately 200 bp PCR fragment containing the intron was digested with PvuII and KpnI, gel-purified, and inserted into PvuII and KpnI-digested pCEP4. The plasmid pCEP4int, with an insert of the correct size, was identified. The structure of the intron was confirmed by DNA sequencing. FLAG-ARP-Phe in pCEP4 was digested with NheI and BamHI. The 400 bp insert was gel-purified and cloned into NheI and BamHI digested pCEP4int to engineer the plasmid FLAG-ARP-Phe in pCEP4int (FIG. 33).

### Example 8

Transient Transfection of Mammalian Cells with ARP and BRP

The HEK 293 EBNA cell line (ATCC, CRL 10852) was used for the production of ARP and BRPfusion proteins, unless stated otherwise. Cell cultures were maintained at 37° C., 5% CO₂, 95% humidity for growth and during procedural incubations. The calcium phosphate precipitation procedure described by Jordan et al. (Nucleic Acids Research. 24: 596-601, 1996) was used for transient transfections except that the growth medium was Dulbecco's modified Eagle's medium F-12 (DMEM/F-12) supplemented with 10% fetal bovine serum (FBS) and 1% L-glutamine. Approximately 1 hr prior to transfection, the growth medium was removed and replaced with transfection medium (DMEM/F-12 supplemented with 2 % FBS, and 1% L-glutamine) and the calcium phosphate precipitated DNA was added. Generally 12.5 μg-25 μg DNA per 100 mm dish was used for a single plasmid transfection. For cotransfections, a mixture of 12.5 μg each plasmid DNA was used. After 4-6 h, the transfection medium was replaced with growth medium. After approximately 24 h, the growth medium was replaced with DMEM/F-12 without supplements (collection medium). After a period of 48-72 h, the collection medium was removed, centrifuged to remove debris, stored for analysis or concentration, and fresh collection medium was added to the cell cultures. After an additional 48-72 h the collection medium was removed, centrifuged, stored, and the cells were discarded. Concentration of the culture medium was done using Centriprep YM-10 (Amicon) according to the protocol specified by the manufacturer.

### Example 9

Stable Transfection of Mammalian Cells with ARP and BRP

Stable transfections with pCEP4-derived plasmids were done as described above for transient transfections, except that collection medium was not used. Approximately 2 days after transfection, the growth medium was replaced with selection medium (DMEM-F12 supplemented with 10% FBS, 1% L-glutamine, and 250 μg/ml hygromycin). Selection medium was replaced every 2-3 days until the cells were confluent and ready to be split for freezing and for production scale-up.

### Example 10

Detection of Secreted GFP Fusion Proteins in the Culture Medium

Both ARP-GFP and BRP-GFP, with their native signal peptide sequences intact, were secreted into the culture medium and could be detected by capture of the fusion proteins on Reacti-Bind Anti-GFP strip plates (Pierce 15188) or by anti-GFP western blot.

To analyze the BRP-GFP fusion protein by western blot, 1 microliter, 2 microliters, and 5 microliters of concentrated media from a transient transfection of BRP-GFP were analyzed. COS-7 cells (African green monkey kidney cells transformed with replication-deficient SV40, American Type Culture Collection Certified Cell Line 1651) were used for this experiment. Cells were plated onto 24-well plates at a density of 100,000 cells per well in 500 microliters of Dulbecco's modified Eagle's medium (DMEM) supplemented 10% v/v with FBS, 1% v/v with 200 mM L-glutamine and 1% v/v with 100 mM pyruvate and cultured under sterile conditions at 37° C., 5% CO₂, 95% humidity. Cells were transfected by lipofection the next day by adding to each well 1 microgram of BRP-GFP plasmid DNA combined with Lipofectamine2000 (Life Technologies, Inc.) in accordance with the manufacturer's instructions. The day after the transfection the cells were switched to serum-free media (DMEM supplemented with 1% v/v L-glutamine and 1% v/v pyruvate) and after 3 more days of culture the media was collected, concentrated using Amicon Centriprep centrifugal concentrators (YM-10) and subjected to SDS-PAGE. Samples (2 microliters) of ARP-GFP from a similar COS transfection using plasmid pEGFP-N2-ARP, as well as samples (1 microliter) from COS-7 cells subjected to transfection conditions without DNA, were also analyzed. The ARP-GFP and control sample volumes were selected so that each contained approximately the same amount of total protein (based on BCA assay of protein content) as was present in the 5 microliter BRP-GFP sample (6.85 micrograms total protein).

Electrophoresis was performed using 10% NuPAGE® Bis-Tris precast gels and reagents from Invitrogen. Samples were combined with deionized water (and for reduced samples, 1 microliter reducing agent (0.5M dithiothreitol), and then 2.5 microliters of 4× NuPAGE® LDS Sample Buffer (Novex NP007) to result in a final volume of 10 microliters. After heating these diluted samples at 70° C. for 5 minutes, the samples were loaded onto a 15-well Invitrogen 10% Bis-Tris NuPAGE gel. Samples of GFP standard (4 nanograms, Clontech) were included as a positive control for the western blot. Broad range prestained markers from Bio-Rad (catalog #72807A) were used along with biotinylated markers from Bio-Rad. Electrophoresis was performed using MOPS running buffer (Novex NP005) at constant voltage (125V) and was stopped when the Serva Blue from the Sample Buffer reached the bottom of the gel. Proteins were transferred from the gel to a PVDF membrane (Novex LC0002) using NuPAGE transfer buffer and a Hoeffer TE 22 Transphor apparatus (using constant current −400 mA). After transfer (confined by migration of prestained markers from the gel to the PVDF) nonspecific binding sites on the membrane were blocked by incubation with 1× casein (Vector Labs SP5020) in water overnight at 4° C. After blocking the membrane was washed three times by incubating each time for 5 minutes while immersed with gentle shaking in TBST (100 mM Tris/0.9% NaCl, pH 7.5+0.1% Tween 20). The membrane was then incubated for 30 minutes at room temperature in a solution of primary antibody (Rockland biotinylated goat anti-GFP 60010615) diluted 1/2000 in TBST, after which the membrane was washed three times in TBST as described above. Detection of the bound antibody was done using reagents from a Vectastain ABC-AP kit (Vector Laboratories AK5001).

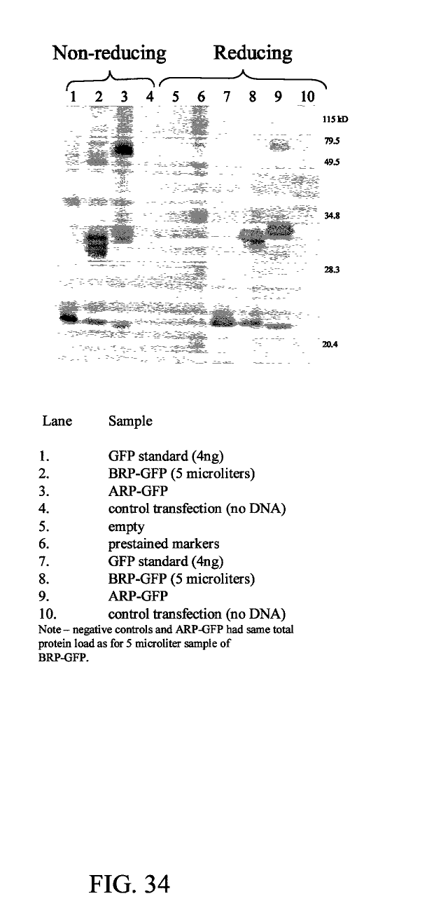

As evident in FIG. 34, both BRP-GFP and ARP-GFP were readily detected in media from transfected cells. Under non-reducing conditions both ARP-GFP and BRP-GFP exhibit oligomeric forms that are diminished or absent in the reduced samples.

### Example 11

Detection of Secreted FLAG Fusion Proteins, and Comparison of FLAG-ARP Expression with and without and Intron

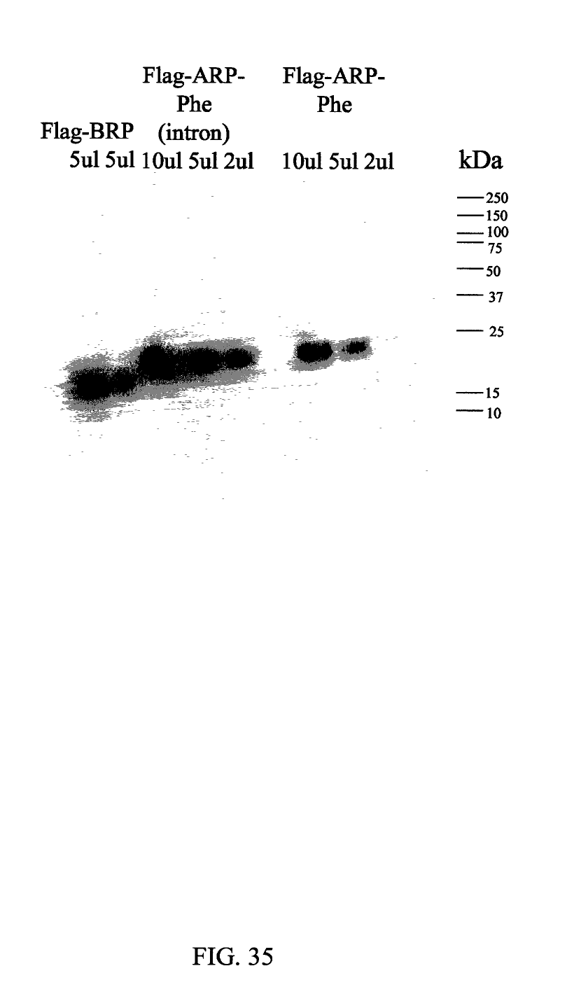

HEK 293 EBNA cells were transiently transfected with the plasmids FLAG- BRPin pCEP4, FLAG-ARP-Phe in pCEP4 and FLAG-ARP-Phe in pCEP4int. Culture supernanant was collected and concentrated. SDS-PAGE (NuPAGE, Invitrogen) was used for protein size separation using 10 μl, 5 μl, and 2 μl aliquots of concentrated supernatant from each ARP transfection, and 5 μl concentrated culture supernatant from the BRPtransfection. The size-fractionated protein was electrotransferred to a PVDF membrane. Following transfer, the membrane was blocked overnight at 4° C. in 5% powdered dry milk. The remaining procedures were done at room temperature. The membrane was washed 5 times with PBST (phosphate buffered saline, 0.05% Tween 20), then treated 1 h in a solution containing anti-FLAG M2 antibody (Sigma-Aldrich #F3165) diluted 1:500 in PBS and 5% bovine serum albumin. After washing 4 times with PBST, the membrane was incubated 1 h in a solution containing goat anti-mouse IgG (H+L) -HRP conjugate (Bio-Rad #170-6516) diluted 1:3000 in PBST+5% powdered dry milk. The membrane was washed 5 times with TBST (1×Tris-buffered saline from 10× concentrate, Bio-Rad #170-6435, 0.05% Tween 20) followed by one wash in Tris-buffered saline pH 9.5 (TBS). HRP was detected with BM Chemiluminescence Substrate (POD) (Roche Molecular Biochemicals 1500694) using the protocol supplied by the manufacturer. A Typhoon 8600 Variable Mode Imager (Molecular Dynamics, Inc. Sunnyvale, Calif.) was used to quantify the signal from the FLAG-ARP-Phe fusion proteins. FLAG- BRPand FLAG-ARP are clearly detectable as bands of Mᵣ 21.5 kDa and Mᵣ 24.2 kDa, respectively (FIG. 35). The signal for the FLAG-ARP-Phe in pCEP4int transfection was 1.9×, 2.8×, and 16× higher than the signal for the FLAG-ARP-Phe in pCEP4 transfection for the 10 μl, 5 μl and 2 μl loading volumes, respectively. These results show that the presence of an intron enhances the expression of the ARP protein, and thus, including an intron or a genomic clone in the expression construct is the best method for production of this protein.

### Example 12

Demonstration of the Formation of an ARP- BRPheterocomplex

Method 1: GFP Capture with AP Detection

Reacti-Bind Anti-GFP strip plates (Pierce 15188) were washed three times with 200 μl PBST. Culture supernatant from HEK 293 EBNA transient transfections with the plasmids encoding AP-ARP+ BRP-GFP (cotransfection), AP- BRP+ARP-GFP (cotransfection), and AP control (single plasmid transfection with pAPtag5) were diluted 1:2, 1:6, and 1:10 in PBS. Duplicate 100 μl aliquots of the undiluted and diluted culture supernatants were placed in the wells and incubated for 20 min at room temperature. After 4 washes with 200 μl PBST, 50 μl distilled water was added to the wells treated with culture supernatant. For a standard curve, 50 μl of serially diluted AP protein (2-fold dilutions from 390 ng/ml to 6.1 ng/ml) were added to clean wells. As an additional control, 50 μl of 1:10 dilutions (in distilled H₂O) of the culture supernatants was added to empty wells at this time to measure total AP. AP assay reagent A (GenHunter® Corporation, Nashville, Tenn.), 50 μl per well, was added to the test samples and the AP standards. The side of the plate was gently tapped to mix the reagents and then incubated at 37° C. for 10 min. The reaction was stopped by the addition of 100 μl 0.5 N NaOH to each well. The optical density at 405 nm was determined in a plate reader. The results of a representative assay are shown in Table 8.

TABLE 8
Detection of ARP-BRPheterocomplexes by GFP capture
and AP detection.

| | | | AP (ng/ml) |
|---|---|---|---|
| | Total AP (ng/ml) | | after anti-GFP Ab capture |
| Transfection | (duplicates) | | (mean ± SD) |
| AP-ARP + BRP-GFP | 1085.7 | 1105.5 | 85.7 ± 6.4 |
| AP-BRP + ARP-GFP | 1017.4 | 947.5 | 249.1 ± 5.7 |
| PAPtag5 | 2762.7 | 2746.7 | 0 |

Method 2. GFP Capture with FLAG Detection

Culture supernatants from HEK 293 EBNA transient transfections with the plasmids encoding FLAG-BRP+ARP-GFP (cotransfection), FLAG-ARP+ BRP-GFP (cotransfection), FLAG- BRP(single plasmid), ARP-GFP (single plasmid), and a no DNA control were used for GFP capture according to the procedure described in method 1. After the capture step and PBST wash, 100 μl of a 1:500 dilution (in PBST) of anti-FLAG M2 monoclonal antibody (Sigma-Aldrich #F3165) was added to each well and incubated 1 h at room temperature, or overnight at 4° C. The wells were washed 4 times with 200 μl PBST and then to each was added 100 μl of a 1:2000 dilution (in PBST) of goat anti-mouse IgG (H+L) -AP Conjugate (Bio-Rad S232425). After washing 5 times with 200 μl PBST, 50 μl of distilled water was added to each well and AP assay reagent A was used as described in method 1 to measure captured protein. The results are shown in Table 9.

TABLE 9
GFP capture with FLAG detection of ARP-BRPheterocomplexes.

| | Abs 405 nm | |
|---|---|---|
| | (Unconcentrated | Abs 405 nm |
| Transfection | medium) | (Concentrated medium) |
| Flag-BRP + ARP-GFP | 0.972 | 1.405 |
| Flag-BRP | 0.271 | 0.286 |
| ARP-GFP | 0.321 | 0.321 |
| No DNA Control | 0.392 | 0.281 |
| Flag-ARP + BRP-GFP | 1.134 | 1.555 |

Method 3. Immunoprecipitation with an Anti-FLAG Monoclonal Antibody and Detection of AP

Culture supernatants (both the first and second 48-72 h collections) from HEK 293 EBNA transient transfections with plasmids encoding AP-ARP+ BRP-GFP (cotransfection), AP- BRP+ ARP-GFP (cotransfection), AP- BRP+FLAG-ARP-Phe (cotransfection), and AP- ARP-Phe+FLAG- BRP(cotransfection) were used for the immunoprecipitation experiments. For each immunoprecipitation reaction, 50 μl MPG beads (CPG Inc.) were washed 3 times in 1 ml of PBST using a magnetic separator. After the last wash, the beads were resuspended in 1 ml PBST. To this was added 5 μl of a 4 μg/μl solution of anti-FLAG M2 monoclonal antibody (Sigma-Aldrich, F3165). The mixture was incubated 20 min at room temperature on a rotator. After washing 5 times in 1 ml PBST, the beads were resuspended in 0.5 ml or 0.1 ml of the test culture supernatant samples and PBST was added to bring each to a total volume of 1 ml. The FLAG tagged protein was captured overnight at 4° C. on a rotator. The beads were washed 5 times with PBST and then resuspended in 50 μl distilled water. Incubation with 50 μl AP assay reagent A and reaction termination with NaOH was done as described in method 1. After stopping the reactions, 100 μl aliquots were removed from each well to separate tubes and diluted in 400 μl distilled water. The optical density at 405 nm was measured in a spectrophotometer. The results are shown in Table 10.

TABLE 10
Detection of ARP-BRPheterocomplexes by immunoprecipitation
with anti-FLAG antibody and detection alkaline phosphatase
activity.

| | | Vol | |
|---|---|---|---|
| | Total AP | culture | AP assay after IP |
| Transfection | (ng/ml) | sup | ng/ml |
| AP-ARP-Phe + BRP- | 730 | 0.5 ml | 0 |
| GFP (first collection) | | 0.1 ml | 0 |
| AP-BRP + ARP-GFP | 570 | 0.5 ml | 0 |
| (first collection) | | 0.1 ml | 0 |
| AP-BRP + flag-ARP- | 1340 | 0.5 ml | 56 |
| Phe (first collection) | | 0.1 ml | 84 |
| AP-ARP-Phe + flag- | 160 | 0.5 ml | 43 |
| BRP (first collection) | | 0.1 ml | 68 |
| AP-ARP-Phe + flag- | 290 | 0.5 ml | 56 |
| BRP (second collection) | | 0.1 ml | 96 |
| AP-BRP + flag-ARP- | 840 | 0.5 ml | 64 |
| Phe (second collection) | | 0.1 ml | 313 |
| AP-ARP + BRP-GFP | 120 | 0.5 ml | 0 |
| (second collection) | | 0.1 ml | 0 |

Taken together, the results show that the BRPand ARP fusion proteins interact to form a heterocomplex. Therefore, it is also expected that the native forms of the proteins would form a heterocomplex with a specific physiological activity.

### Example 13

Small-Scale Production of FLAG- BRP

HEK 293 EBNA cells stably transfected with the expression plasmid FLAG- BRPin pCEP4 were expanded into four T-175 flasks (Falcon Cat#353112) in growth medium. When approximately confluent, the cells were trypsinized (Gibco Cat#25300-054), pooled, and used to seed a 6320 cm² cell factory (Nunc Cat#164327). The cells were fed as needed with growth medium until they reached confluence, when the growth medium was replaced with production medium (DMEM/F-12 containing 1 mg/l insulin, 12.24 mg/l ferric citrate and 0.0068 mg/l selenium). The production medium was removed every 2-3 days and replaced with fresh production media (1500-2250 ml) to produce several lots. Each lot was filtered through a Gelman 0.45 μm mini-capsule filter (Gelman Cat#12123) immediately after harvesting, then placed in Nalgene PETG bottles (Cat#2019-0500 and 2019-1000) and stored at −80° C.

### Example 14

Recognition of a Human BRP Homocomplex

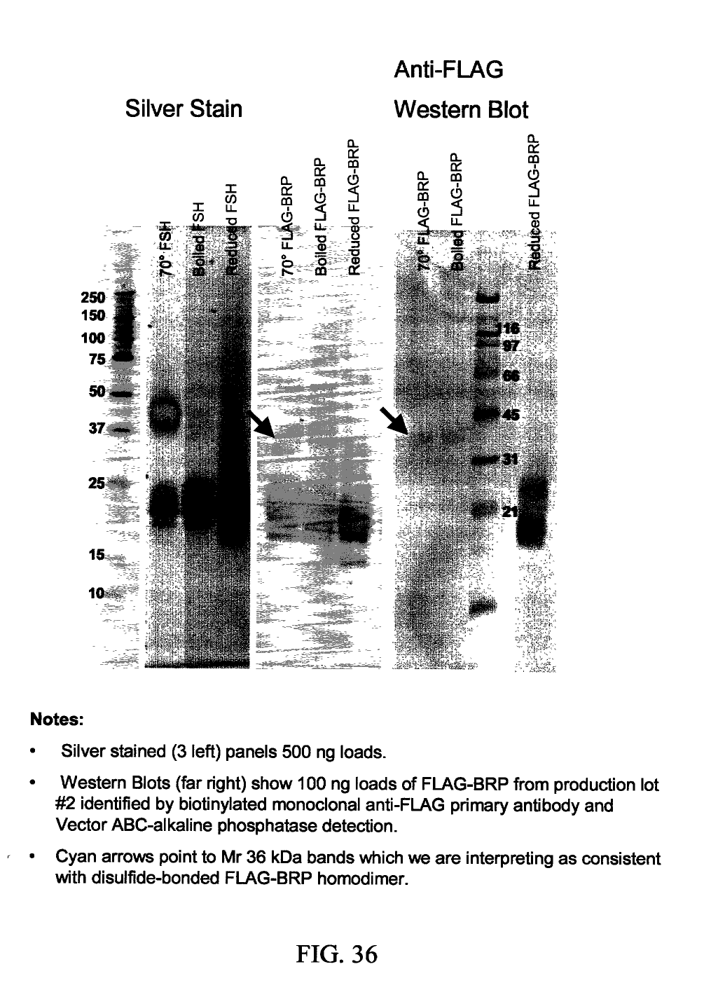

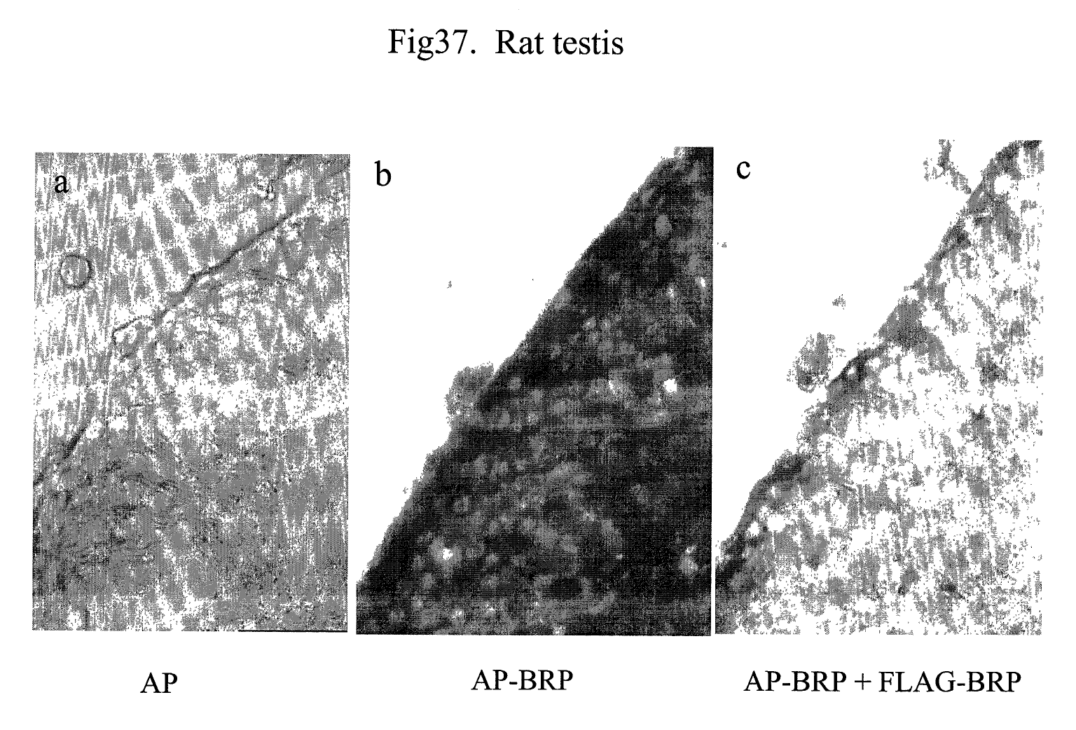

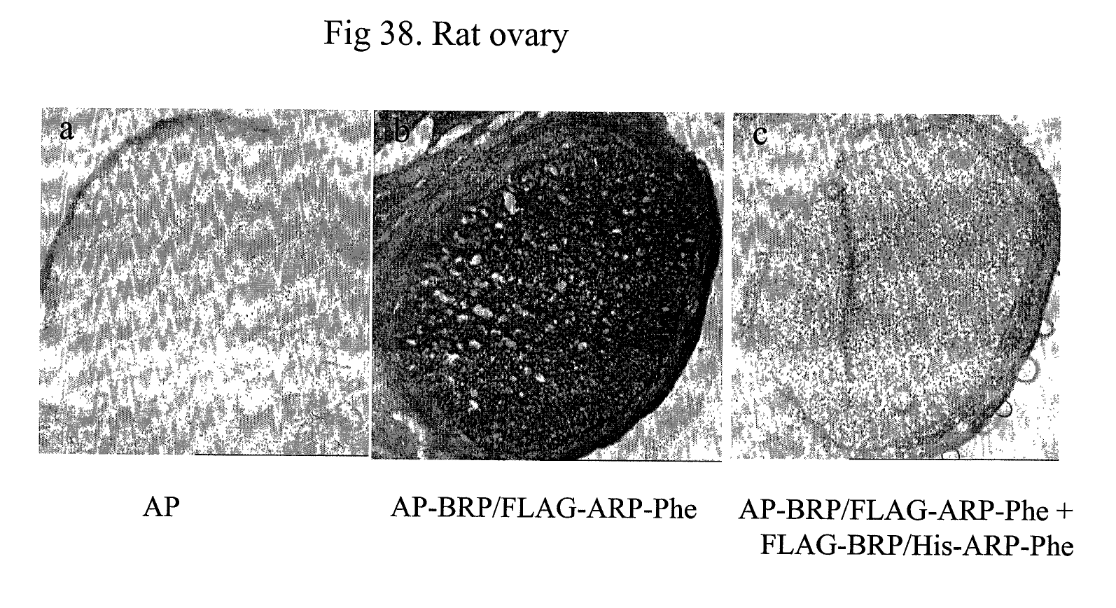

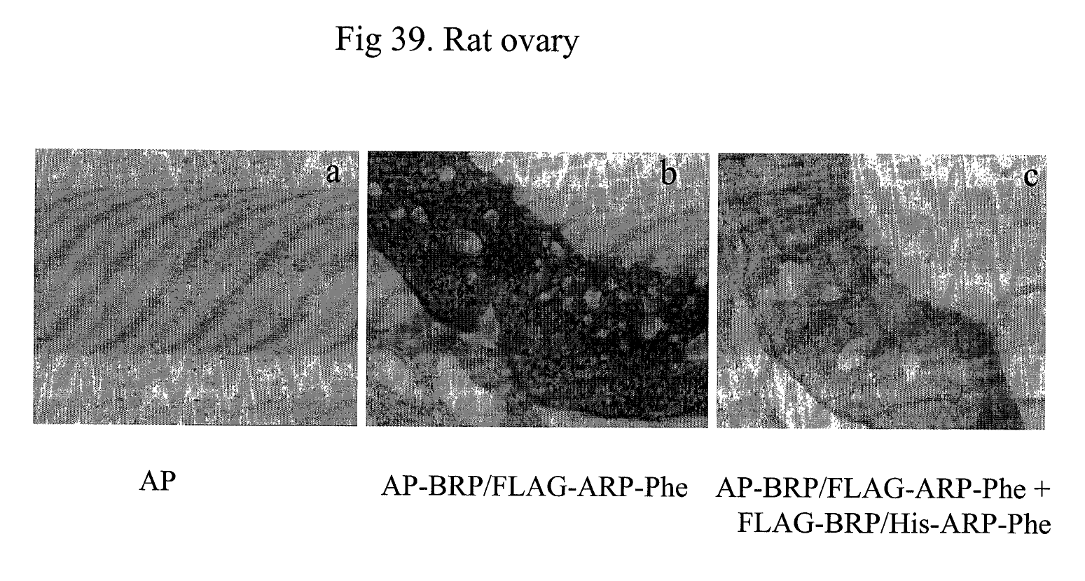

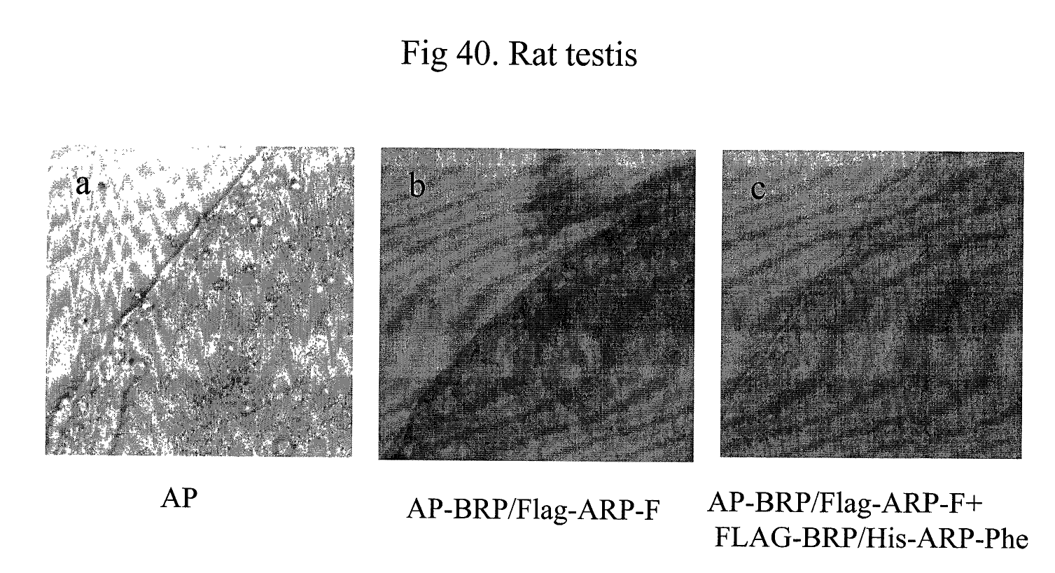

After concentration of the crude culture supernatant from two small scale production lots, FLAG- BRP was immunoaffinity-purified to approximately 75% homogeneity using ANTI-FLAG-agarose affinity gel (Sigma, #A-1205). The purified protein was analyzed by SDS-PAGE and Anti-FLAG specific western blot using the Tris-Tricine buffer system of Schagger and von Jagow (Anal Biochem. Nov. 1, 1987;166(2):368-79). FIG. 36shows images of a silver stained gel and an Anti-FLAG western blot aligned with respect to apparent molecular weight. The pretreatment conditions, 70° for 10 min, boiling for 2 min and boiling for 2 min in the presence of 2% β-mercaptoethanol were selected because under those conditions FSH exists, respectively, as heterodimer, dissociated heterodimer, and reduced dissociated heterodimer (See FSH comparators on silver-stained gel).

Detection of a reduction-sensitive band at Mᵣ 36 kDa in both lots by silver staining suggests that at least a portion of the purified FLAG- BRPprotein exists as a covalent homodimer. It is unlikely that the 36 kDa band is an artifact of the transient expression system, or of the immunoaffinity purification method.

### Example 14

In situ Histomchemistry of ARP/BRP

Animal and Tissue Preparation

Mature 60-day-old Sprague-Dawley rats were used. The animals were allowed free access to food and water. Tissue sections were prepared according to a published procedure (Flanagan et al., Methods in Enzymology, Vol. 327 p19-31.)

In situ Analysis of AP-BSARP or AP-B5 Binding to Rat Tissues

Sections were washed two times in a 10 mM Tris, pH 7.6, 5 mM MgCl₂ buffer for 5 min at room temperature and then preincubated in a blocking buffer (10 mM Tris, pH 7.6, 5 mM MgCl₂, 2.5% BSA) at room temperature for one hour. Sections were then incubated with one of the following treatments:

1. conditioned media from 293 cells transfected with pAPtag5 (GenHunter) (AP)

2. conditioned media from 293 cells transfected with AP-BRP in pAPtag5 (AP-BRP)

3. conditioned media from 293 cells co-transfected with AP-BRP in pAPtag5 and FLAG-ARP-Phe in pCEP4 (AP-BRP/FLAG-ARP-Phe)

4. conditioned media from 293 cells co-transfected with AP-BRP in pAPtag5 and FLAG-ARP-Phe in pCEP4, plus conditioned media from 293 cells co-transfected with His-ARP-Phe+FLAG-BRP (AP-BRP/FLAG-ARP-Phe+FLAG-BRP/His-ARP-Phe)

5. conditioned media from 293 cells transfected with AP-BRP in pAPtag5 plus partially purified FLAG-BRP (AP-BRP+FLAG-BRP).

Incubations with AP proteins were performed at room temperature overnight in the blocking buffer. After incubation, the sections were then washed in cold blocking buffer six times, fixed for 30 seconds in 20 mM HEPES buffer (pH 7.5) containing acetone (60%) and formaldehyde (3%). The fixed sections were then washed and heated at 65° C. for 30 min in a HS buffer (150 mM NaCl in 20 mM HEPES, pH 7.5) to inactivate endogenous alkaline phosphatase activity. After completely removing the HS buffer, the sections were stained for AP activity using GenHunter AP Assay Reagent S to detect the cell surface bound AP activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid molecule encoding a polypeptide comprising an amino acid sequence that is at least 75% identical to SEQ ID NO: 2 or 4, or the complement of said nucleic acid molecule.

2. The isolated nucleic acid molecule of claim 1, wherein said nucleic acid molecule hybridizes under stringent conditions to a nucleic acid sequence complementary to a nucleic acid molecule comprising the sequence of nucleotides of SEQ ID NO: 1 or 3, or the complement of said nucleic acid molecule.

3. The isolated nucleic acid molecule of claim 1, wherein said nucleic acid molecule encodes a polypeptide comprising the amino acid sequence of SEQ ID NO: 2 or 4 or an amino acid sequence comprising one or more conservative substitutions in the amino acid sequence of SEQ ID NO: 2 or 4.

4. The nucleic acid molecule of claim 1, wherein said nucleic acid molecule encodes a polypeptide comprising the amino acid sequence of SEQ ID NO: 2 or 4, or the complement of said nucleic acid molecule.

5. The nucleic acid molecule of claim 1, wherein said nucleic acid molecule comprises the sequence of nucleotides of SEQ ID NO: 1 or 3, or the complement of said nucleic acid molecule.

6. The nucleic acid molecule of claim 1, wherein said nucleic acid molecule has the nucleotide sequence of a cDNA.

7. The nucleic acid molecule of claim 1, wherein said nucleic acid molecule comprises contiguous nucleotides encoding the amino acid sequence WEKPI (SEQ ID NO: 5).

8. An isolated nucleic acid molecule, wherein said nucleic acid molecule:
(a) encodes a polypeptide having the amino acid sequence of SEQ ID NO: 2; and
(b) comprises contiguous nucleotides encoding the amino acid sequences. WEKPI (SEQ ID NO: 5);
or the complement of said nucleic acid molecule.

9. A nucleic acid molecule less than 100 nucleotides in length and comprising at least 6 contiguous nucleotides of SEQ ID NO: 1 or a complement thereof.

10. A nucleic acid vector comprising the nucleic acid molecule of claim 1.

11. A host cell comprising the isolated nucleic acid molecule of claim 1.

12. An isolated polypeptide at least 80% identical to a polypeptide selected from the group consisting of:
a) a polypeptide comprising an amino acid sequence of SEQ ID NO: 2 or 4;
b) a fragment of a polypeptide comprising an amino acid sequence of SEQ ID NO: 2 or 4, wherein the fragment comprises at least 6 contiguous amino acids of SEQ ID NO: 2;
c) a derivative of a polypeptide comprising an amino acid sequence of SEQ ID NO: 2 or 4;
d) an analog of a polypeptide comprising an amino acid sequence of SEQ ID NO: 2 or 4;
e) a homolog of a polypeptide comprising an amino acid sequence of SEQ ID NO: 2 and 4; and
f) a naturally occurring allelic variant of a polypeptide comprising an amino acid sequence of SEQ ID NO: 2 or 4, wherein the polypeptide is encoded by a nucleic acid molecule that hybridizes to a nucleic acid molecule of SEQ ID NO: 1 or 3, under stringent conditions.

13. The polypeptide of claim 12, wherein the polypeptide, or fragment thereof,
a) binds to a glycoprotein hormone receptor;
b) binds to a LGR orphan G-protein-coupled receptor;
c) binds to a glycoprotein hormone; or
d) binds to a cystine knot protein.

14. The polypeptide of claim 12, wherein the polypeptide, or fragment thereof, is glycosylated at one or more sites.

15. The polypeptide of claim 12, wherein the polypeptide, or fragment thereof, is not glycosylated.

16. The polypeptide of claim 12, wherein the polypeptide, or fragment thereof, comprises a cystine knot domain.

17. A protein multimer comprising a first polypeptide according to claim 12, and a second polypeptide.

18. A protein multimer comprising an ARP polypeptide and a second polypeptide.

19. The protein multimer according to claim 17 or 18, wherein said second polypeptide is identical to the first polypeptide.

20. The protein multimer according to claim 17, wherein said second polypeptide is an apha glycoprotein subunit.

21. The protein multimer according to claim 18, wherein said second polypeptide is an beta glycoprotein subunit.

22. The protein multimer according to claim 17 or 18, wherein said second polypeptide is a cystine knot protein.

23. The protein multimer according to claim 17, wherein said second polypeptide comprises the amino acid sequence of SEQ ID NO: 18.

24. The protein multimer according to claim 17 or 18, wherein said protein multimer is a dimer

25. An antibody that selectively binds to the polypeptide of claim 12, and fragments, homologs, analogs, and derivatives of said antibody.

26. An antibody that selectively binds to the protein multimer of claim 17 or 18, and fragments, homologs, analogs, and derivatives of said antibody.

27. A method of producing a BRP polypeptide, said method comprising the step of culturing the host cell of claim 11 under conditions in which the nucleic acid molecule is expressed.

28. A method of detecting the presence of the polypeptide of claim 12 in a sample, comprising contacting the sample with a compound that selectively binds to the polypeptide of claim 12 and determining whether the compound bound to the polypeptide of claim 12 is present in the sample.

29. A method of detecting the presence of a nucleic acid molecule of claim 1 in a sample, the method comprising contacting the sample with a nucleic acid probe or primer that selectively binds to the nucleic acid molecule and determining whether the nucleic acid probe or primer bound to the nucleic acid molecule of claim 1 is present in the sample.

30. A method for modulating the activity of the polypeptide of claim 12, the method comprising contacting a cell sample comprising the polypeptide of claim 12 with a compound that binds to said polypeptide in an amount sufficient to modulate the activity of the polypeptide.

31. A method of treating or preventing a reproductive disorder in a subject, the method comprising administering to a subject method comprising administering to a subject in need thereof a therapeutic selected from the group consisting of:
a) a ARP/BRP nucleic acid;
b) a ARP/BRP polypeptide and
c) a ARP/BRP antibody;
wherein said therapeutic is administered in an amount sufficient to treat or prevent said reproductive disorder in said subject.

32. A method of treating or preventing a reproductive disorder in a subject, the method comprising administering to a subject method comprising administering to a subject in need thereof a therapeutic comprising a protein multimer of claim 17 or 18 wherein said therapeutic is administered in an amount sufficient to treat or prevent said reproductive disorder in said subject.

33. A pharmaceutical composition comprising a therapeutically or prophylactically effective amount of a therapeutic selected from the group consisting of:
a) a ARP/BRP nucleic acid;
b) a ARP/BRP polypeptide and
c) a ARP/BRP antibody
and a pharmaceutically acceptable carrier.

34. A pharmaceutical composition comprising a therapeutically or prophylactically effective amount of a therapeutic selected from the group consisting of the protein multimer of claim 17 or 18 and a pharmaceutically acceptable carrier.

35. A kit comprising in one or more containers, comprising a therapeutically or prophylactically effective amount of the pharmaceutical composition of claim 33 or 34.

36. A method for screening for a modulator of activity or of latency or predisposition to a reproductive disorder, said method comprising:
a) administering a test compound to a test animal at increased risk for a pathology associated with the polypeptide of claim 1, wherein said test animal recombinantly expresses a ARP/BRP polypeptide;
b) measuring the activity of said polypeptide in said test animal after administering the compound of step (a); and
c) comparing the activity of said protein in said test animal with the activity of said polypeptide in a control animal not administered said polypeptide, wherein a change in the activity of said polypeptide in said test animal relative to said control animal indicates the test compound is a modulator of latency of, or predisposition to, a reproductive disorder.

37. The method of claim 35, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a reproductive disorder in a subject, the method comprising:
a) measuring the amount of a ARP/BRP polypeptide or ARP/BRP multimer in a sample from the subject; and
b) comparing the amount of said polypeptide in step (a) to the amount of the polypeptide present in a control sample,
wherein an alteration in the level of the polypeptide or multimer in step (a) as compared to the control sample indicates a disease condition.

39. A method for determining the presence of or predisposition t o a reproductive disorder in a subject, the method comprising:
a) measuring the amount of a ARP/BRP nucleic acid in a sample from the mammalian subject; and
b) comparing the amount of said nucleic acid in step (a) to the amount of the nucleic acid present in a control sample,
wherein an alteration in the level of the nucleic acid in step (a) as compared to the control sample indicates a disease condition.

40. A method for expressing an ARP/BRP polyppetide as a product if an endogenous gene in a cell, wherein the polypeptide is expressed at a modified level ina comparison to the wild type cell, the method comprising;
(a) transfecting the cell with a DNA constuct, the DNA constrict comprising a transcription regulatory element in operative connection to the endogenous gene, thereby producing a recombinant cell and/or
(b) transfecting the cell with a DNA constuct, the DNA constrict comprising a amplifiable gene and a DNA targeting sequence capable of inserting the amplifiable gene in operative connection to the endogenous gene, thereby producing a recombinant cell a
(c) culturing the recombinant cell, and if desired, selecting cells containing multiple copies of the endogenous gene.


SEQUENCE LISTING (107 sequences)

SEQ ID NO: 1 - DNA, 447 bp, Homo sapiens
ttgaaggcagccagatctgttaaactctgtcctttccctctccggaagagcagcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctga

SEQ ID NO: 2 - protein, 129 aa, Homo sapiens
MKLAFLLLGPMALLLLAGYGCLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 3 - DNA, 381 bp, Xenopus sp.
atgaacaagaagagggtgatgttccctgtcctgcagcttctggttttagccctgtgtctcagcaccgctgcaggatccaatataagtctgagaacgttcattggatgtgctgtgagggaattcacattcttagcaaagaaacctggctgcagaggtctgcgtgtgactactgatgcctgctgggggcgctgtgagacctgtgagaagccatccctagatcctccgtacatagaagcccaccacagagtctgcacttacaatgaaactaaactggttactgtaatactgccaaactgcagcccagacattgacccattctttacctacccagttgccattagatgtgactgtgacatgtggtccacttctactacagaatgt

SEQ ID NO: 4 - protein, 127 aa, Xenopus sp.
MNKKRVKFPVLQLLVLALCLSTAAGSNISLRTFIGCAVREFTFLAKKPGCRGLRVTTDACWGRCETCEKPSLDPPYIEAHHRVCTYNETKLVTVILLPNCSPDIDPFFTYPVAIRCDCMWSTSTTEC

SEQ ID NO: 5 - protein, 5 aa, Homo sapiens
WEKPI

SEQ ID NO: 6 - protein, 141 aa, Homo sapiens
MEMLQGLLLLLLLSMGGAWASREPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTCDHPQLSGLLFL

SEQ ID NO: 7 - protein, 129 aa, Homo sapiens
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 8 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 9 - protein, 138 aa, Homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 10 - protein, 23 aa, Homo sapiens
MKLAFLLLGPMALLLLAGYGCLG

SEQ ID NO: 11 - protein, 20 aa, Homo sapiens
MEMFQGLLLLLLLSMGGTWA

SEQ ID NO: 12 - protein, 19 aa, Homo sapiens
ETWEKPILEPPYIEAHHRV

SEQ ID NO: 13 - protein, 166 aa, Artificial Sequence
  Description of Artificial Sequence Fusion Protein
MEMFQGLLLLLLLSMGGTWASKEPLRPRCRPINATLAVEKEGCPVCITVNTTICAGYCETWEKPILEPPYIEAHHRVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 14 - protein, 143 aa, Artificial Sequence
  Description of Artificial Sequence Fusion Protein
MKLAFLLLGPMALLLLAGYGCLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECTVRGLGPSYCSFGEMKE

SEQ ID NO: 15 - protein, 21 aa, Homo sapiens
CETWEKPILEPPYIEAHHRVC

SEQ ID NO: 16 - protein, 19 aa, Homo sapiens
ETWEKPILEPPYIEAHHRV

SEQ ID NO: 17 - DNA, 754 bp, Homo sapiens
cggcacgaggcagcaggaggcacaggaaaactgcaagccgctctgttcctgggcctcggaagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttaacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctctttgcccttacccctaaataaagcaagcagttcttg

SEQ ID NO: 18 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEILTARACQCDMCRLSRY

SEQ ID NO: 19 - DNA, 596 bp, Mus musculus
cggcacgtaggggagtcttcagttgctgttggactgtcctttgcagatgcccatggcaccacgagtcttgctcctttgcctgctgggcctggcagtcactgaagggcatagcccagagacagccatcccaggctgccacttgcaccccttcaatgtgacggtgcgcagtgatcgcctcggcacttgccagggctcccacgtggcacaggcctgtgtaggacactgtgagtctagtgctttcccttcccggtactctgtgctggtggccagtggctatcggcacaacatcacctcttcctcccagtgctgcaccatcagcagcctcagaaaggtgagggtgtggctgcagtgcgtggggaaccagcgtggggagcttgagatctttactgcaagggcctgccagtgtgatatgtgccgtttctcccgctactagtccccgaagctcaggctccggtcctgccactgacatgtcatgggtatctcaaactcggggctctgaccctctttatcgtctgtgaagatgaggttggccctctcagcagtctccttgctacattctccttcgctcctgtcctcaataaagcaagcaatgcttg

SEQ ID NO: 20 - protein, 128 aa, Mus musculus
MPMAPRVLLLCLLGLAVTEGHSPETAIPGCHLHPFNVTVRSDRLGTCQGSHVAQACVGHCESSAFPSRYSVLVASGYRHNITSSSQCCTISSLRKVRVWLQCVGNQRGELEIFTARICQCDMCRFSRY

SEQ ID NO: 21 - DNA, 844 bp, Rattus norvegicus
gggggagggaggggccgaagtggccagggttggtatgatccccagccatgagagacatcccaggggacagtgcatagaaggatggcatacacacaagtggctgctcattgccttccagagtagctgaggcaaggaagcaagcaccccacacattcccacccaaggcagagaggatcaacagtgccacccaggcacacctcacagtcggaagacccagaagcctggcttgctgggggagagacacaactgcaaagacttcccttcccacccactccttttcagatgcccatggcacctcgagtcttgctcttctgcctgctgggtctggcagtcactgaagggcatggcctggaggcagccgtcccaatcccaggctgccacttgcacccctttaacgtgacagtgcgaagtgatcgccatggcacctgccagggctcccatgtggcacaggcgtgtgtaggacactgtgagtctagtgctttcccttctcggtactctgtgctggttgccagtggctatcgacacaacatcacctctgtctctcagtgctgtaccatcagcagccttaaaaaggtgagggtgtggctgcactgcgtggggaaccagcgtggggagctcgagatcttcacggctagggcctgccagtgtgatatgtgccgtctctcccgctactaggccccgaagctcaggcctccagtcctgccactgataggtcgtgcttctctcagaccagccctctttggagtctgaagatggggcttcgcctctgtttacctggcctcctcagcagtctcactgctgctttctccttcacccctgtcctcaataaagcaggcagtgcttg

SEQ ID NO: 22 - protein, 129 aa, Rattus norvegicus
MPMAPRVLLFCLLGLAVTEGHGLEAAVPIPGCHLHPFNVTVRSDRHGTCQGSHVAQACGHCESSAFPSRYSVLVASGYRHNITSVSQCCTISSLKKVRVWLHCVGNQRGELEIFTARACQCDMCRLSRY

SEQ ID NO: 23 - DNA, 1224 bp, Homo sapiens
agatggcgaagaaaattccagggaagggagaatcactgcacagagggctgacacacaggtcctttccagagacagctgctcacactcacacccatacacacacacacacacacacaaaggcagatacagggaaaaggcagcaccattcaggcacacctcacctgtcagaccagccagccctggctcactcacctggaatgcagtatttaaagaactcgccatcccacctgcacacccacgtagagacatctccccactgtgtttcagatgcctatggcgtcccctcaaaccctggtcctctatctgctggtcctggcagtcactgaagcctggggccaggaggcagtcatcccaggctgccacttgcaccgtgagtacctctgggaccggagggctaggagcagtggaggttctgggtgggagcaaagagctgacagagtggacggtggggcaggcagcaccctaaagggccccacactgaggcacaggcaacgggagctggggcgaggcaaaccttggcagaggcgccgtctactgcttgcctatctccttctagccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtgaggagggcccgggcccggtggatggacgctggggtcgcgggaagaccagagagatggagatcctagacagccctgagaaaggggactgcagcacggactcccctctcccgcaggtcaaagtacagctgcagtgtgtggggagccggagggaggagctcgagatcttcacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctct

SEQ ID NO: 24 - protein, 6 aa, Homo sapiens
LHPFNV

SEQ ID NO: 25 - protein, 6 aa, Homo sapiens
LKKVKV

SEQ ID NO: 26 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 27 - protein, 129 aa, Homo sapiens; identical to SEQ ID NO: 7

SEQ ID NO: 28 - protein, 23 aa, Homo sapiens
MPMASPQTLVLYLLVLAVTEAWG

SEQ ID NO: 29 - protein, 22 aa, Mus musculus
MPMAPRVLLLCLLGLAVTEGHS

SEQ ID NO: 30 - protein, 22 aa, Rattus norvegicus
MPMAPRVLLFCLLGLAVTEGHG

SEQ ID NO: 31 - protein, 107 aa, Artificial Sequence
  Description of Artificial Sequence Consensus Sequence
CRPGCRPTNYTISVEKEECPVCITINTTICAGYCYTRDPVYKSPLLPLPQRVCTYGEWSYETARLPGCPPGVDPHFTYPVALSCYCGKCNTDTTDCTVLSLRPDSCS

SEQ ID NO: 32 - protein, 99 aa, Homo sapiens
TFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 33 - protein, 107 aa, Homo sapiens
LRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCD

SEQ ID NO: 34 - protein, 107 aa, Anguilla anguilla
LLLPCEPINETISVEKDGCPKCLVFQTSICSGHCITKDPSYKSPLSTVYQRVCTYRDVRYETVRLPDCRPGVDPHVTFPVALSCDCNLCTMDTSDCAIQSLRPDFCM

SEQ ID NO: 35 - protein, 107 aa, Fundulus heteroclitus
QLPRCQLLNQTISLEKRGCSGCHRVETTICSGYCATKDPNYKTSYNKAIQHVCTYGDLYYKTFEFPECVPGVDPVVTYPVALSCRCGGCAMATSDCTFESLQPDFCM

SEQ ID NO: 36 - protein, 109 aa, Artificial Sequence
  Description of Artificial Sequence Consensus Sequence
ATKKRPKCRPTNVTIYVEKEGCTSCKTVNTTICAGYCYTKDPVYKDGRRLLIQCVCCYPDVTYETKVLPGCPNGVDPTKTYPVALSCHCGKCNTDNTDCTRESLHPDSC

SEQ ID NO: 37 - protein, 102 aa, Homo sapiens
NLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 38 - protein, 109 aa, Homo sapiens
KEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTC

SEQ ID NO: 39 - protein, 104 aa, Homo sapiens
NSCELTNITIAIEKEECRFCISINTAWCAGYCYTRDLVYKDPARPKIQKTCTFKELVYETVRVPGCAHHADSLYTYPVATQCHCGKCDSDSTDCTVRGLGPSYC

SEQ ID NO: 40 - protein, 109 aa, Homo sapiens
REPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTC

SEQ ID NO: 41 - protein, 109 aa, Ctenolepisma lineata
GGSLLLPCEPINETISVEKDGCPKCLVFQTSICSGHCITKDPSYKSPLSTVYQRVCTYRDVRYETVRLPDCRPGVDPHVTFPVALSCDCNLCTMDTSDCAIQSLRPDFC

SEQ ID NO: 42 - protein, 109 aa, Ctenolepisma lineata
QSSFLPPCEPVNETVAVEKEGCPKCLVFQTTICSGHCLTKEPVYKSPFSTVYQHVCTYRDVRYETVRLPDCPPGVDPHITYPVALSCDCSLCTMDTSDCTIESLQPDFC

SEQ ID NO: 43 - protein, 109 aa, Fundulus heteroclitus
AAFQLPRCQLLNQTISLEKRGCSGCHRVETTICSGYCATKDPNYKTSYNKAIQHVCTYGDLYYKTFEFPECVPGVDPVVTYPVALSCRCGGCAMATSDCTFESLQPDFC

SEQ ID NO: 44 - protein, 105 aa, Rana catesbeiana
RHVCHLANATISAEKDHCPVCITFTTSICTGYCQTMDPVYKTALSSFKQNICTYKEIRYDTIKLPDCLPGTDPFFTYPVALSCYCDLCKMDYSDCTVESSEPDVC

SEQ ID NO: 45 - protein, 111 aa, Anguilla anguilla
AGQVLSICSPVDYTLYVEKPECDFCVAINTTICMGFCYSLDPNVVGPAVKRLVVQRGCTYQAVEYRTAELPGCPPHVDPRFSYPVALHCTCRACDPARDECTHRASADGDR

SEQ ID NO: 46 - DNA, 23 bp, Artificial Sequence
  Description of Artificial Sequence PCR Primer Sequence
tcgatgatgggcttcaatatagg

SEQ ID NO: 47 - DNA, 26 bp, Artificial Sequence
  Description of Artificial Sequence PCR Probe Sequence
cctgggagaaacccattctggaaccc

SEQ ID NO: 48 - DNA, 22 bp, Artificial Sequence
  Description of Artificial Sequence PCR Primer Sequence
gcctcagatggtctcacactcc

SEQ ID NO: 49 - DNA, 29 bp, Artificial Sequence
  Description of Artificial Sequence PCR Primer Sequence
ctcgaggcctccagtgggaacctgcgcac

SEQ ID NO: 50 - DNA, 31 bp, Artificial Sequence
  Description of Artificial Sequence PCR Primer Sequence
gggcccggatcctcagatggtctcacactcc

SEQ ID NO: 51 - DNA, 24 bp, Artificial Sequence
  Description of Artificial Sequence PCR Primer Sequence
gctagcatgaagctggcattcctc

SEQ ID NO: 52 - DNA, 23 bp, Artificial Sequence
  Description of Artificial Sequence PCR Primer Sequence
tatcgatggtctcacactccgtg

SEQ ID NO: 53 - DNA, 48 bp, Artificial Sequence
  Description of Artificial Sequence Synthetic Oligonucleotide
ctagtctcgaggctgcagttgctgactacaaagacgatgacgacaagg

SEQ ID NO: 54 - DNA, 44 bp, Artificial Sequence
  Description of Artificial Sequence Synthetic Oligonucleotide
ccttgtcgtcatcgtctttgtagtcagcaactgcagcctcgaga

SEQ ID NO: 55 - DNA, 27 bp, Artificial Sequence
  Description of Artificial Sequence PCR Primer Sequence
tttgctagcaccatgtctgcacttctg

SEQ ID NO: 56 - DNA, 27 bp, Artificial Sequence
  Description of Artificial Sequence PCR Primer Sequence
tttggatcctcagatggtctcacactc

SEQ ID NO: 57 - DNA, 19 bp, Artificial Sequence
  Description of Artificial Sequence PCR Primer Sequence
aggaggcagtcatcccagg

SEQ ID NO: 58 - DNA, 18 bp, Artificial Sequence
  Description of Artificial Sequence PCR Primer Sequence
tgccttggcggtcacttc

SEQ ID NO: 59 - DNA, 24 bp, Artificial Sequence
  Description of Artificial Sequence PCR Probe Sequence
tgccacttgcaccccttcaatgtg

SEQ ID NO: 60 - DNA, 17 bp, Artificial Sequence
  Description of Artificial Sequence PCR Primer Sequence
aggcagccgtcccaatc

SEQ ID NO: 61 - DNA, 22 bp, Artificial Sequence
  Description of Artificial Sequence PCR Primer Sequence
gatcacttcgcactgtcacgtt

SEQ ID NO: 62 - DNA, 22 bp, Artificial Sequence
  Description of Artificial Sequence PCR Probe Sequence
caggctgccacttgcacccctt

SEQ ID NO: 63 - DNA, 31 bp, Artificial Sequence
  Description of Artificial Sequence PCR Primer Sequence
ttttaagcttagtgatgcctatggcgtcccc

SEQ ID NO: 64 - DNA, 25 bp, Artificial Sequence
  Description of Artificial Sequence PCR Primer Sequence
ttttgaattcgtagcgagagaggcg

SEQ ID NO: 65 - DNA, 22 bp, Artificial Sequence
  Description of Artificial Sequence PCR Primer Sequence
tttgagatcttcacggccaggg

SEQ ID NO: 66 - DNA, 17 bp, Artificial Sequence
  Description of Artificial Sequence Synthetic Oligonucleotide
ctagaggaattcgggcc

SEQ ID NO: 67 - DNA, 9 bp, Artificial Sequence
  Description of Artificial Sequence Synthetic Oligonucleotide
cgaattcct

SEQ ID NO: 68 - DNA, 31 bp, Artificial Sequence
  Description of Artificial Sequence PCR Primer Sequence
ttttctagaacaggaggcagtcatcccaggc

SEQ ID NO: 69 - DNA, 28 bp, Artificial Sequence
  Description of Artificial Sequence PCR Primer Sequence
ttttgaattcctagtagcgagagaggcg

SEQ ID NO: 70 - DNA, 52 bp, Artificial Sequence
  Description of Artificial Sequence PCR Primer Sequence
agttgctgactacaaagacgatgacgacaagcaggaggcagtcatcccaggc

SEQ ID NO: 71 - DNA, 42 bp, Artificial Sequence
  Description of Artificial Sequence PCR Primer Sequence
cccgtttaaacggatcctcagtagcgagagaggcgacacatg

SEQ ID NO: 72 - DNA, 74 bp, Artificial Sequence
  Description of Artificial Sequence PCR Primer Sequence
tttgctagccaccatgtctgcacttctgatcctagctcttgttggagctgcagttgctgactacaaagacgatg

SEQ ID NO: 73 - DNA, 42 bp, Artificial Sequence; identical to SEQ ID NO: 71
  Description of Artificial Sequence PCR Primer Sequence

SEQ ID NO: 74 - DNA, 26 bp, Artificial Sequence
  Description of Artificial Sequence PCR Primer Sequence
ggtaccaaggtagccttgcagaagtt

SEQ ID NO: 75 - DNA, 26 bp, Artificial Sequence
  Description of Artificial Sequence PCR Primer Sequence
cagctggtaattgaactgggagtgga

SEQ ID NO: 76 - DNA, 18 bp, Artificial Sequence
  Description of Artificial Sequence PCR Primer Sequence
gggccttcggatcaccac

SEQ ID NO: 77 - DNA, 22 bp, Artificial Sequence
  Description of Artificial Sequence PCR Primer Sequence
cagcatgaagctggcattcctc

SEQ ID NO: 78 - DNA, 6240 bp, Homo sapiens
aggaatctctggatgcctgtgttggagtttgtgggcatttacaatttctgggctcattttccctgaaatgctaggagcaaggtccctttgatagtgacaaatgcatggttggctgtgccattgaaggcagccagatctgttaaactctgtcctttccctctccggaagagcagcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggaggtgagttgctaagttgtgcagatgacagtgtcttctaggccagcagcttgggtctgattcttaagagttcactttttaaatgatatgaggtagagctgggacatctgccctttcctgtggacttaaaaaaccaaaacaaaactatgattggcatcttccaaaagtgatttgaaaaacatgatgttgcccctctaacaaagcattgataaggttaagaatttggtttacattgtgtctatgtatctgggaatcatctctgggaggtcaagatgtactgttctacccgttttacagatgacatggagggattcaagggagagtggctgcaaagtcacgtagagcgtcagtgtaaagctgggaatcaatctgtggttcaagcttgtgacccaaactcctccctatgtttcctcattttggataaattagccagtttccaagaaagaggccctgagctgaagggtgagcgttggtcccagtgaagggtgagaccccttcactgcctcttctgcagcccttttcctcctcaagtctctgggagccctctggggttatcactgacggatccattaagttccttcatattcaattatacctggcctttttagagacatttaatttaaagtggagataacactctcaaacaaagttaaaatcctattgggctaagaggagctgtttgagtgatgaagaggaagagagctattcagcaccccagcagatcacattacgtagtgactgtgggctcttccccctgaggcctgcccacttggtaaccaatgaagtgctgtctctgatcttgtcactccctggcccaaaaaccttgaatgtccacacactactacagattcaataactaactttcaaggtgctcagcaatatggcgtctgcctgctttcctggagacagcacattttcttactctggccttggtaagtgactttcaaaggttttatcaaatagcccttatggatctcattttgttccttccctcatatcccttctccttcccatctgtcattatcatatttattcctgatgcctatctgcagtgccagctccctttctgggccttttttgacttgcaggtaagcccttgactatgctctacttttcgtcttacttcctcccccaccacacgcgtgatttaaattttttcaggacagaggttcattcttataaccttcacagcttttgtcaagatgtcgtgtatgaacaaggcattcaatacacatttgttggttgactgggatggacctccccctggagctgtagatcctccagcctaatggaaggccatttagaatcacacttgcactgtgagtggacactgccattgggaaaaatagccttctctttggggacccagagggtaacctgctcttgcttaggtacaattacggccctgtgaatggaattgggtcatagtgatgaaatctccaaattggatgaaactactctatcaaagtagttttcttttgcctcattcaggggcttgagccctactagcccaatgaaaatcgggttttgctaagtagactttgcctgtcaattggcagcaaattcacctggggcacttggcacctcctcctgttcagggactggcctggcagggcctctccctgttcgcatctagtgtctgggctatttgaagccctctctgtgccaaatcctcaaactcctgcttccgttcgattcagcccatcttctcttctttttaaaaactgatgaatgtctttaattggatcatggtcacccataggaggtcaggaactgtgctctcactggaaagatggaaacaccaaaaccgttaaagaacaagattctccctgatgttagccagctttcattcatgtcttgactgtgttatgaaaagggaggttacctatagaaaataaataaaagaatgagattcattttcccagcaatctgaaagtttctgcgctataaagcacttgattttttggtgggggggatcttaactgaaagcatgtctgaaaataaggatgttcatgatgacaggctggctggatttacatttgaaggttgttgaaaatagctattcctcataatctgggtatagagttgccagatttagcaaacaaacaaacagacaaacaaaataaaacaaaaccaatcccctccccacagaaacccaaactgaaataaaaccagaaaaccaggaagcccaggtaaattggaatttaagataaataataaataaatttttagcgtaagtctgtctgtctcatacagtatttgggatgacttatactaaaaaattatgtatctgaaaatgaaattttacggggcgtttggtctgcctaggttcccagagtactaatggtaagaggacttaaagcaaatacgggaaggtaggagaaaacagttcaggacaaattcagctcttctggtctttgtcaaaggcaaggctggccgggcgtggtggctaacacctgtaatctcagcactttgggaggctgtggtgggtggataatgaggtcaggagttcgagaccagcctggccagtttttagtaaagaggtgagttaaaccctgtctctactaaaaatacaaaaattagccgggcatggtggtatgcacctgtagtcccagctacttgggaggctgaggcagaagacttgcttgaacccaggaggtggaggttacagtgagccaagatcatgccactatactccagcctggcgacagagtgagactccatctcaaaaaaaaaaaaaaaagaaaaaagaaaaaaaaaaggtaaggctgctattttcatgacattcatgcaagaacatcttgagttacatatgtatatatattcttttttgcctagaacaaagaagaaccaaaaagcaaaggtactgtcatttgaaagcttgttattatttacattactttcttataataattgcactaataagaacaatggattggctgggcgtggtggctcacgcctgtaatcccagcactttgggaggccgaggcaggcagatcacgaggtcaggaaatcgagaccatcctggctaacatggtgaaaccctgtctctactaaaaatacaaaaaatgagccaggcgtggtggtgggtgcctgtagtcccgggaggctgaggcaggagaatggcgtgaacccgggaggcggagattgcaatgagctgagattgcgccactgaactccagcctgggagacagcaagactccgtctcaaaaaaaaaaaaaatggattgcattttttgaacatttactttgttctagacattgtgcattgcgtatatcatcttaccttatctctcaaacaatggtgggaggtagctattttgttttacagaggaggaaacttgagtcttcaggaagttaagtggattttccaaggtctccagcaagtggcagaacagggactcaagctccttagttctgactgcagggctcgagattttaactccagctaggtgctgatattttttctgatctgtgtgttctgtttatcaaaattgtctttgaacttaagatttataaaaggtgaaggaaggaaatgaatctttttgatgatcagaacagtgcacagagtattcgggaacctgtcttgtaatgttttctttcattgattcaatgacaaatagttattgaaactctcccggggtctgttttgggtacttgaggcacagtgggcaaaaatctctgtcctaaaagagcttactttctagagtgggaggaatatcacacgaatgaaaggtagactacgtcgtgtggtattgatcagtgctgtggtggaaaataaagcaagatgggggatgggaagtttctgggcatggagatggaatgttgcaattttaaataggatggtcaggaaatgcttccctgagagggtgacattctaacaaaaacccaaggttggtgaaagagtgaatcatacgggagaagaatgttccaggcagaaggaacggtaagtgcaaaggccctgagctggggctgttcctggtgggtcagaggagcaataaggagaccgccgtgagcctagtgaggaagtcagtgaggtgggaatggttgcaggcatttcagaaggtagagttgcagagaaggtgatgtaggtcttgaaggtgatcataaggtctttgatgtttgttctgagtgagatgggaaatcactggggctttgggcagaggagtgacatgatctgacttaggtttaaacaggatcactcagggccgctgtgttgcaaatagattgtagggagtaaaaatggaagaggggagaccagttagaaggtatttgcaatgactaagatgattcatttgctgactatgcatggagcacttgctgtgtgctatggtctctcctgggagcttagaatatggtcttgagtgaaatcagcttcttgctttcaggagtttgttttctactgggagacgacagagcaacaagtaaatcaacgaataacaagttaatttctgatagtgataaatgatactaaaaaactgaaacaagatcatatgttctaatgaattctctgttttctatctatggggacagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccacctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaacaaaacccatgtactagtcttccttaccccagaggaattccagctgcaagcttctttagggaaaatgctcccttccccttttaactgagcaattatctacacaagaaataagactgctcagatatacaaagagagtagcttcaatgaaaagatgtttggatttggataattcttttccctagcaaaattcgctagctcccttaagagtcttaataaagaggctacgttgggattaaaagaaaaaaaaacagaaataaaatatgtaactaatagctatctcatttagccttaaaaacttattaaactaaactcatgttttagagtatgatgttctcccaaagctatggcaaaatggccaatcacaagtattcttccccatttatcatattttcaatttaagttgtaacttactaaactcagaaattttatatgcgtttaggggtaaaactgcatggctggctcagaggaaaaagcctgtgattttctagctcctgcctctctaaaatcttacagtagctaattctgtggctggaaaaaacctccaaaactctaatgttatgcaaatgtctttaattctggcatttttggggttgaatttaaccttgttcctttttcataatgtgccaagaaaacctatattaatgccaataaagcatgtcctctgtcttttggattcatgacaacattcaagaaagtctttttaattcttagtatacttggagta

SEQ ID NO: 79 - DNA, 1224 bp, Homo sapiens; identical to SEQ ID NO: 23

SEQ ID NO: 80 - DNA, 490 bp, Artificial Sequence
  Description of Artificial Sequence Fusion Protein
cactttgcctttctctccacaggtgtccactcccagttcaattaccagctgctagcgtcgaccatgtctgcacttctgatcctagctcttgttggagctgcagttgctcatcatcaccatcaccatggtgacgatgacgataagcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactagtcgacggatccagacatgataagat

SEQ ID NO: 81 - protein, 130 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 82 - DNA, 420 bp, Homo sapiens
cgaattcgcccttcagcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggcaagggcg

SEQ ID NO: 83 - protein, 106 aa, Artificial Sequence
  Description of Artificial Sequence Fusion Protein
ASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 84 - DNA, 420 bp, Artificial Sequence
  Description of Artificial Sequence Fusion Protein
ggactagtcctgcaggtttaaacgaattcgcccttctcgaggcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggatccgggcccaagggcgaattcgcggccgctaaattcaattcgccctatagtgagt

SEQ ID NO: 85 - protein, 131 aa, Artificial Sequence
  Description of Artificial Sequence Fusion Protein
LEPYTACDLAPPAGTTDAAHPGYLEASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 86 - DNA, 420 bp, Artificial Sequence
  Description of Artificial Sequence Fusion Protein
cctggagccctacaccgcctgcgacctggcgccccccgccggcaccaccgacgccgcgcacccgggttatctcgaggcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggatccgggcccgaacaaaaact

SEQ ID NO: 87 - protein, 387 aa, Artificial Sequence [sequence omitted]

SEQ ID NO: 88 - DNA, 1210 bp, Artificial Sequence
  Description of Artificial Sequence Fusion Protein
gcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatcgataaagggcaattctgcagatatccagcacagtggcggccgctcgagtctagaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattaaacccgctgatcagcctcgactgtgccttctagttgc

SEQ ID NO: 89 - protein, 129 aa, Artificial Sequence
  Description of Artificial Sequence Fusion Protein
MSALLILALVGAAVADYKDDDDKASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 90 - DNA, 490 bp, Artificial Sequence
  Description of Artificial Sequence Fusion Protein
ccgttgacgcaaatgggcggtaggcgtgtacggtgggaggtctatataagcagagctcgtttagtgaaccgtcagaattaattcaccatgtctgcacttctgatcctagctcttgttggagctgcagttgctgactacaaagacgatgacgacaaggcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggatcccgggtgg

SEQ ID NO: 91 - protein, 129 aa, Homo sapiens; identical to SEQ ID NO: 18

SEQ ID NO: 92 - DNA, 490 bp, Homo sapiens
ggcgaattgggtaccgggccccccctcgaggtcgacggtatcgataagcttagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttaacggccagggcctgccagtgtgacatgtgtcgcctctctcgctacgaattcctgcagcccgggggatccactagttctagagcggccgccacc

SEQ ID NO: 93 - protein, 129 aa, Homo sapiens; identical to SEQ ID NO: 18

SEQ ID NO: 94 - DNA, 390 bp, Homo sapiens
atgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttaacggccagggcctgccagtgtgacatgtgtcgcctctctcgctactag

SEQ ID NO: 95 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 96 - DNA, 490 bp, Homo sapiens
ggcgaattgggtaccgggccccccctcgaggtcgacggtatcgataagcttagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctacgaattcctgcagcccgggggatccactagttctagagcggccgccacc

SEQ ID NO: 97 - protein, 386 aa, Artificial Sequence
  Description of Artificial Sequence Fusion Protein
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEILTARACQCDMCRLSRYEFCSRRYRGPGIHRPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 98 - DNA, 1190 bp, Artificial Sequence
  Description of Artificial Sequence Fusion Protein
agcttagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttaacggccagggcctgccagtgtgacatgtgtcgcctctctcgctacgaattctgcagtcgacggtaccgcgggcccgggatccaccggccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatc

SEQ ID NO: 99 - protein, 165 aa, Artificial Sequence [sequence omitted]

SEQ ID NO: 100 - DNA, 560 bp, Artificial Sequence
  Description of Artificial Sequence Fusion Protein
gccgcctgcctggagccctacaccgcctgcgacctggcgccccccgccggcaccaccgacgccgcgcacccgggttatctcgaggaagcgctctctctagaacaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctacgaattcgggcccgaacaaaaactcatctcagaagaggatctgaatagcgccgtcgaccatcatcatcatcatcattgagtttaaacccgctgatcagcctcgactgtgccttctagttgccagccatctgttgtttgccc

SEQ ID NO: 101 - protein, 129 aa, Artificial Sequence
  Description of Artificial Sequence Fusion Protein
MSALLILALVGAAVADYKDDDDKQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 102 - DNA, 420 bp, Artificial Sequence
  Description of Artificial Sequence Fusion Protein
agctgctagccaccatgtctgcacttctgatcctagctcttgttggagctgcagttgctgactacaaagacgatgacgacaagcaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactgaggatccagacatgata

SEQ ID NO: 103 - DNA, 69 bp, Artificial Sequence
  Description of Artificial Sequence PCR Primer Sequence
ctcttgttggagctgcagttgctcatcatcaccatcaccatggtgacgatgacgataagcaggaggcag

SEQ ID NO: 104 - DNA, 39 bp, Artificial Sequence
  Description of Artificial Sequence PCR Primer Sequence
tttggatccgtcgactagtagcgagagaggcgacacatg

SEQ ID NO: 105 - DNA, 65 bp, Artificial Sequence
  Description of Artificial Sequence PCR Primer Sequence
tttgctagcgtcgaccatgtctgcacttctgatcctagctcttgttggagctgcagttgctcatc

SEQ ID NO: 106 - DNA, 39 bp, Artificial Sequence; identical to SEQ ID NO: 104
  Description of Artificial Sequence PCR Primer Sequence

SEQ ID NO: 107 - protein, 133 aa, Artificial Sequence
  Description of Artificial Sequence Fusion Protein
MSALLILALVGAAVAHHHHHHGDDDDKQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY